(19) United States
(12) Patent Application Publication
(10) Pub. No.: US 2007/0167447 A1
(43) Pub. Date: Jul. 19, 2007

(54) Indole acetamides as inhibitors of the hepatitis c virus ns5b polymerase

(76) Inventors: Salvatore Avolio, Casoria (Naples) (IT); Marcello Di Filippo, Genzano (IT); Steven Harper, Albano Laziale (Rome) (IT); Frank Narjes, Ariccia (Rome) (IT); Barbara Pacini, Albano Laziale (Rome) (IT); Marco Pompei, Rome (IT); Michael Rowley, Axa (Rome) (IT); Ian Stansfeld, Ariccia (Rome) (IT)

Correspondence Address: MERCK AND CO., INC, P O BOX 2000, RAHWAY, NJ, 07065-0907, US

(21) Appl. No.: 10/551,564

(22) PCT Filed: Apr. 2, 2004
(86) PCT No.: PCT/GB04/01437

§ 371 Date: Jun. 5, 2006

(30) Foreign Application Priority Data
Apr. 4, 2003 (GB) 0307891.2

(51) Int. Cl. A61K 31/5377; A61K 31/506; A61K 31/496; A61K 31/4709; A61K 31/454; A61K 31/405; C07D 413/02

(52) U.S. Cl. 514/232.8; 514/269; 514/414; 514/419; 514/323; 514/254.09; 546/201; 548/465; 548/495; 544/373; 544/143; 544/315; 514/364; 548/124; 514/310; 546/148; 514/300; 546/113

(57) ABSTRACT

The present invention relates to indole and azaindole compounds of formula (I): wherein X¹, X², X³, X⁴, A¹, Ar¹, R¹, R² and n are as defined herein, and pharmaceutically acceptable salts thereof, useful in the prevention and treatment of hepatitis C infections.

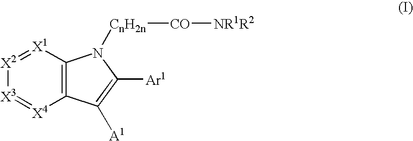

The present invention relates to indole and azaindole compounds, to pharmaceutical compositions containing them, to their use in the prevention and treatment of hepatitis C infections and to methods of preparation of such compounds and compositions.

Hepatitis C (HCV) is a cause of viral infections. There is as yet no adequate treatment for HCV infection but it is believed that inhibition of its RNA polymerase in mammals, particularly humans, would be of benefit International patent applications WO 01/47883, WO 02/04425 and WO 03/000254 suggest fused ring compounds as possible inhibitors of HCV polymerase and illustrate thousands of possible benzimidazole derivatives that possess HCV polymerase inhibitory properties. However, these patent applications do not describe or reasonably suggest the preparation of any benzimidazole or azabenzimidazole substituted on all three available sites on the fused imidazole ring. WO 03/010140 and WO 03/010141 suggest further fused ring compounds as possible inhibitors of HCV polymerase and illustrate thousands of possible compounds all of which possess complex esterified side chains. The corresponding acids are suggested as intermediates only and not as HCV polymerase inhibitors. In particular none of these patent applications describe an indole or azaindole in which the indole nitrogen is substituted by an alkylamide residue.

The present invention provides compounds of the formula (I) 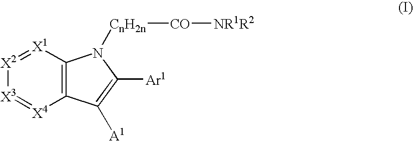
wherein:

Ar¹ is a moiety containing at least one aromatic ring and possesses 5-, 6-, 9- or 10-ring atoms optionally containing 1, 2 or 3 heteroatoms independently selected from N, O and S, which ring is optionally substituted at any substitutable position by groups Q¹ and Q²;

Q¹ is halogen, hydroxy, C₁₋₄ alkyl, C₁₋₄ alkoxy, aryl, heteroaryl, CONR^{c}R^{d}, CₘH₂ₘNR^{c}R^{d}, —O—(CH₂)₂₋₄R^{c}R^{d}, —O—CₘH₂ₘCONR^{c}R^{d}, —O—CₘH₂ₘ aryl, —O—CₘH₂ₘ heteroaryl, —O—CR^{e}R^{f};

R^{c} and R^{d} are each independently selected from hydrogen, C₁₋₄ alkyl and C(O)C₁₋₄ alkyl;

or R^{c}, R^{d} and the nitrogen atom to which they are attached form a heteroaliphatic ring of 4 to 7 ring atoms, where said ring is optionally substituted by halogen, hydroxy, C₁₋₄ alkyl or C₁₋₄ alkoxy;

m is 0, 1, 2 or 3

R^{e} and R^{f} are each independently selected from hydrogen and C₁₋₄ alkoxy;

or R^{e} and R^{f} are linked by a heteroatom selected from N, O and S to form a heteroaliphatic ring of 4 to 7 ring atoms, where said ring is optionally substituted by halogen, hydroxy, C₁₋₄ alkyl or C₁₋₄alkoxy;

and wherein said C₁₋₄ alkyl, C₁₋₄ alkoxy and aryl groups are optionally substituted by halogen or hydroxy;

Q² is halogen, hydroxy, C₁₋₄ alkyl or C₁₋₄ alkoxy, where said C₁₋₄ alkyl and C₁₋₄ alkoxy groups are optionally substituted by halogen or hydroxyl;

or Q¹ and Q² may be linked by a bond or a heteroatom selected from N, O and S to form a ring of 4 to 7 atoms, where said ring is optionally substituted by halogen, hydroxy, C₁₋₄ alkyl or C₁₋₄ alkoxy;

A¹ is C₁₋₆ alkyl, C₂₋₆ alkenyl, where said C₁₋₆ alkyl and C₂₋₆ alkenyl groups are optionally substituted by C₁₋₄ alkoxy or up to 5 fluorine atoms, or a non-aromatic ring of 3 to 8 ring atoms where said ring may contain a double bond and/or may contain a O, S, SO, SO₂ or NH moiety and where said ring is optionally substituted by one or two alkyl groups of up to 2 carbon atoms or by 1 to 8 fluorine atoms, or a non-aromatic bicyclic moiety of 4 to 8 ring atoms which ring may be optionally substituted by fluorine or hydroxy;

X¹ is N or CR^{a};

X² is N or CR³;

X³ is N or CR⁴;

X⁴ is N or CR^{b};

with the proviso that X² and X³ are not both N;

R^{a} and R^{b} are each independently selected from hydrogen, fluorine, chlorine, C₁₋₄ alkyl, C₂₋₄ alkenyl or C₁₋₄ alkoxy, where said C₁₋₄ alkyl, C₂₋₄ alkenyl and C₁₋₄ alkoxy groups are optionally substituted by hydroxy or fluorine;

one of R³ or R⁴ is hydrogen, halogen, C₁₋₄ alkyl, C₁₋₄ alkoxy, CN, CO₂H, CO₂C₁₋₄ alkyl, aryl, heteroaryl or C(O)NR⁹R¹⁰, where said C₁₋₄ alkyl, C₁₋₄ alkoxy, aryl and heteroaryl groups are optionally substituted by hydroxy or fluorine;

R⁹ is hydrogen or C₁₋₄ alkyl;

R¹⁰ is hydrogen, C₁₋₄ alkyl, C₂₋₄ alkenyl or (CH₂)₀₋₃R¹² or SO₂R¹¹;

R¹² is NR^{h}Rⁱ, OR^{h}, aryl, heteroaryl, indolyl or Het;

R^{h} and Rⁱ are each independently selected from hydrogen and C₁₋₄ alkyl;

Het is a heteroaliphatic ring of 4 to 7 ring atoms, which ring may contain 1, 2 or 3 heteroatoms selected from N, O or S or a group S(O), S(O)₂, NH or NC₁₋₄ alkyl;

R¹¹ is C₁₋₄ alkyl, C₂₋₄ alkenyl or (CH₂)₀₋₃R¹³;

R¹³ is aryl, heteroaryl, C₁₋₄ alkyl, C₃₋₈ heteroalkyl, Het or NR^{m}Rⁿ, wherein Het is as hereinbefore defined, R^{m} and Rⁿ are each independently selected from hydrogen, C₁₋₄ alkyl and CO₂(CH₂)₀₋₃aryl, and wherein R¹³ is optionally substituted by halogen, C₁₋₄ alkyl or NR^{o}R^{p}, wherein R^{o} and R^{p} are each independently selected from hydrogen and C₁₋₄ alkyl;

and where R¹⁰ is optionally substituted by hydroxy, fluorine, chlorine, C₁₋₄ alkyl, ═O, CO₂H or CO₂C₁₋₄ alkyl;

or R⁹, R¹⁰ and the nitrogen atom to which they are attached form a heteroaliphatic ring of 4 to 7 ring atoms, where said ring is optionally substituted by halogen, hydroxy, ═O, C₁₋₄ alkyl or C₁₋₄ alkoxy;

the other of R³ and R⁴ is hydrogen, fluorine, chlorine, C₁₋₄ alkyl, C₂₋₄ alkenyl or C₁₋₄ alkoxy, where said C₁₋₄ alkyl, C₂₋₄ alkenyl and C₁₋₄ alkoxy groups are optionally substituted by hydroxy or fluorine;

n is 1, 2, 3 or 4;

R¹ and R² are each independently selected from hydrogen, C₁₋₆ alkyl, C₂₋₆ alkenyl, C₁₋₆ alkynyl, C₁₋₄ alkoxy, C₃₋₈ cycloalkylC₁₋₄ alkyl, (CH₂)₀₋₃R¹⁴;

R¹⁴ is aryl, heteroaryl, NR^{q}R^{r}, Het, where Het is as hereinbefore defined;

R^{q} and R^{r} are each independently selected from hydrogen and C₁₋₄ alkyl;

or R ^{q}, R^{r} and the nitrogen atom to which they are attached form a heteroaliphatic ring of 4 to 7 ring atoms;

and R¹ and R² are optionally substituted by hydroxy, C₁₋₄ alkyl, ═O, C(O)C₁₋₄ alkyl or C₃₋₈ cycloalkyl;

or R¹, R² and the nitrogen atom to which they are attached form a heteroaliphatic ring of 4 to 7 ring atoms, which ring optionally contains 1, 2 or 3 additional heteroatoms selected from O and S or a group S(O), S(O)₂, NH or NR^{s}, where R^{s} is C₁₋₄ alkyl or heteroaryl, or said heteroaliphatic ring is fused to or substituted by a spiro-fused five- or six-membered nitrogen-containing heteroaliphatic ring, which heteroaliphatic ring is optionally substituted by hydroxy, C₁₋₄ alkyl, C₁₋₄ alkoxy, (CH₂)₀₋₃NR^{t}R^{u}, aryl, heteroaryl, or a —CH₂— or —CH₂CH₂— alkylene bridge, where aryl and heteroaryl are optionally substituted by hydroxy, C₁₋₄ alkyl or C₁₋₄ alkoxy;

R^{t} and R^{u} are each independently selected from hydrogen, C₁₋₄ alkyl and C(O)C₁₋₄ alkyl,

or R^{t}, R^{u} and the nitrogen atom to which they are attached form a heteroaliphatic ring of 4 to 7 ring atoms optionally substituted by C₁₋₄ alkyl; and pharmaceutically acceptable salts thereof.

A preferred class of compounds for formula (I) is that wherein Ar¹ is a five- or six-membered aromatic ring optionally containing 1, 2 or 3 heteroatoms selected from N, O and S, which ring is optionally substituted at any substitutable position by groups Q¹ and Q² as hereinbefore defined.

A further preferred class of compounds of formula (I) is that wherein Ar¹ is a six-membered aromatic ring optionally containing 1, 2 or 3 heteroatoms selected from N, O and S, which ring is optionally substituted by groups Q¹ and Q² as hereinbefore defined. Preferably, Ar¹ is a six-membered ring optionally containing 1 or 2 N atoms, such as phenyl, 1-pyridyl, 2-pyridyl, 3-pyridyl, pyridazinyl, pyrimidinyl and pyrazinyl, which ring is optionally substituted by groups Q¹ and Q² as hereinbefore defined. More preferably, Ar¹ is phenyl, 2-pyridyl or 3-pyridyl, optionally substituted by groups Q¹ and Q² as hereinbefore defined. Most preferably, Ar¹ is phenyl, optionally substituted by groups Q¹ and Q² as hereinbefore defined.

A further preferred class of compounds of formula (I) is that wherein Ar¹ is a five-membered aromatic ring optionally containing 1, 2 or 3 heteroatoms selected from N, O and S, which ring is optionally substituted by groups Q¹ and Q² as hereinbefore defined. Preferably, Ar¹ is a five-membered ring containing 1 or 2 heteroatoms selected from N, O and S, such as 2-furanyl, 3-furanyl, 2-thienyl, 3-thienyl, pyrazolyl and imidazolyl, which ring is optionally substituted by groups Q¹ and Q² as hereinbefore defined. More preferably, Ar¹ is 3-furanyl, 2-thienyl or pyrazolyl, optionally substituted by groups Q¹ and Q² as hereinbefore defined. Most preferably, Ar¹ is 3-furanyl, optionally substituted by groups Q¹ and Q² as hereinbefore defined.

Preferably, Q¹ is halogen, hydroxy, C₁₋₄ alkyl or C₁₋₄ alkoxy. More preferably, Q¹ is fluorine, chlorine, methyl or methoxy.

Preferably, Q² is halogen, more preferably fluorine.

Preferably, Ar¹ is unsubstituted.

A preferred class of compounds of formula (I) is that wherein A¹ is C₁₋₆ alkyl, C₂₋₆ alkenyl or C₃₋₈ cycloalkyl, where A¹ is optionally substituted by halogen, hydroxy, C₁₋₄ alkyl or C₁₋₄ alkoxy. Preferably, A¹ is C₃₋₈ cycloalkyl, preferably cyclopentyl or cyclohexyl, more preferably cyclohexyl, optionally substituted by halogen, hydroxy, C₁₋₄ alkyl or C₁₋₄ alkoxy.

Preferably, A¹ is unsubstituted or substituted by fluorine, chlorine, methyl or methoxy. More preferably, A¹ is unsubstituted.

A preferred class of compounds of formula (I) is that wherein X¹ is CR^{a} wherein R^{a} is as hereinbefore defined. Preferably, R^{a} is hydrogen, fluorine, methyl, methoxy or trifluoromethyl. More preferably, R^{a} is hydrogen.

A preferred class of compounds of formula (I) is that wherein X⁴ is CR^{b} wherein R^{b} is as hereinbefore defined. Preferably, R^{b} is hydrogen, fluorine, methyl, methoxy or trifluoromethyl. More preferably, R^{b} is hydrogen.

A preferred class of compounds of formula (I) is that wherein X² is CR³ wherein R³ is as hereinbefore defined. Preferably, R³ is hydrogen, CO₂H, heteroaryl, or C(O)NR⁹R¹⁰.

When R³ is heteroaryl, preferably heteroaryl is tetrazolyl or 1,2,4oxadiazol-3-yl, optionally substituted by hydroxy.

When R³ is C(O)NR⁹R¹⁰, preferably R⁹ is hydrogen or methyl, more preferably hydrogen.

When R³ is C(O)NR⁹R¹⁰, preferably R¹⁰ is SO₂R¹¹ wherein R¹¹ is as hereinbefore defined. Preferably, R¹¹ is C₁₋₄ alkyl, phenyl, benzyl, trifluoromethyl, CH₂CF₃, methoxyphenyl, pyridyl, thienyl, C₂H₄ phenyl and C₂H₄NR^{m}Rⁿ.

Preferably, R^{m} is hydrogen or methyl.

Preferably, Rⁿ is hydrogen, methyl or CO₂CH₂Ph.

A preferred class of compounds of formula (I) is that wherein n is 1 or 2. Preferably, n is 1.

A preferred class of compounds of formula (I) is that wherein X³ is CR⁴ wherein R⁴ is as hereinbefore defined. Preferably, R⁴ is hydrogen, fluorine, chlorine, C₁₋₄ alkyl, C₂₋₄ alkenyl or C₁₋₄ alkoxy, where said C₁₋₄ alkyl, C₂₋₄ alkenyl and C₁₋₄ alkoxy groups are optionally substituted by hydroxy and fluorine. More preferably, R⁴ is hydrogen, fluorine, methyl, methoxy or trifluoromethyl. Most preferably, R⁴ is hydrogen.

A preferred class of compounds of formula (I) is that wherein R¹ is hydrogen, C₁₋₆ alkyl, or (CH₂)₀₋₃R¹⁴ wherein R¹⁴ is as hereinbefore defined.

When R¹ is (CH₂)₀₋₃R¹⁴, preferably R¹ is CH₂Het wherein Het is as hereinbefore defined. Preferably, Het is a five- or six-membered heteroaliphatic ring containing a group NC₁₋₄ alkyl, preferably NMe.

Preferably, R¹ is C₁₋₆ alkyl, more preferably C₁₋₄ alkyl, most preferably methyl.

A preferred class of compounds of formula (I) is that wherein R² is hydrogen, C₁₋₆ alkyl or C₂₋₆ alkenyl. Preferably, R² is hydrogen or methyl. More preferably, R² is methyl.

A further preferred class of compounds of formula (I) is that wherein R¹, R² and the nitrogen atom to which they are attached form a five- or six-membered heteroaliphatic ring, which ring optionally contains one additional oxygen atom or a group NR^{s} wherein R^{s} is as hereinbefore defined, which ring is optionally substituted by (CH₂)₀₋₃NR^{t}R^{u} wherein R^{t} and R^{u} are as hereinbefore defined, preferably NR^{t}R^{u} or CH₂NR^{t}R^{u}.

Preferably, R^{t} is C₁₋₄ alkyl, more preferably methyl.

Preferably, R^{u} is C₁₋₄ alkyl, more preferably methyl.

Preferably, the heteroaliphatic ring is pyrrolidinyl, piperidinyl, piperazinyl or morpholinyl, optionally substituted by (CH₂)₀₋₃NR^{t}R^{u} wherein R^{t} and R^{u} are as hereinbefore defined.

One favoured group of compounds of the present invention are of formula (Ia) and pharmaceutically acceptable salts thereof: 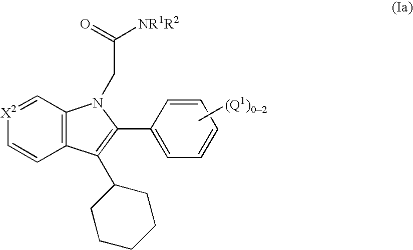
wherein Q¹, X², R¹ and R² are as defined in relation to formula (I).

Preferably, X² is CR³ wherein R³ is as hereinbefore defined. Preferably, R³ is CO₂H, heteroaryl or C(O)NR⁹R¹⁰.

Preferably, R⁹ is hydrogen or methyl, more preferably hydrogen.

Preferably, R¹⁰ is SO₂R¹¹ wherein R¹¹ is as hereinbefore defined. Preferably, R¹¹ is C₁₋₄alkyl, phenyl, benzyl, trifluoromethyl, CH₂CF₃, methoxyphenyl, pyridyl, thienyl or (CH₂)₂ phenyl.

Preferably, R¹ is hydrogen, C₁₋₆ alkyl or CH₂ Het wherein Het is as hereinbefore defined.

Preferably, R² is hydrogen or C₁₋₆ alkyl, more preferably hydrogen or methyl, most preferably methyl.

Preferably R¹, R² and the nitrogen atom to which they are attached form a five- or six-membered heteroaliphatic ring, which ring optionally contains one additional oxygen atom or a group NR^{s} wherein R^{s} is as hereinbefore defined, which ring is optionally substituted by (CH₂)₀₋₃ NR^{t}R^{u}, wherein R^{t} and R^{u} are as hereinbefore defined, preferably NR^{t}R^{u} or CH₂NR^{t}R^{u}.

When any variable occurs more than one time in formula (I) or in any substituent, its definition on each occurrence is independent of its definition at every other occurrence.

As used herein, the term “alkyl” or “alkoxy” as a group or part of a group means that the group is straight or branched. Examples of suitable alkyl groups include methyl, ethyl, n-propyl, i-propyl, n-butyl, s-butyl and t-butyl. Examples of suitable alkoxy groups include methoxy, ethoxy, n-propoxy, i-propoxy, n-butoxy, s-butoxy and t-butoxy.

The cycloalkyl groups referred to herein may represent, for example, cyclopropyl, cyclobutyl, cyclopentyl or cyclohexyl. A suitable cycloalkylalkyl group may be, for example, cyclopropylmethyl.

As used herein, the terms “alkenyl” and “alkynyl” as a group or part of a group means that the group is straight or branched. Examples of suitable alkenyl groups include vinyl and allyl. Suitable alkynyl groups are ethynyl and propargyl.

When used herein, the term “halogen” means fluorine, chlorine, bromine and iodine. Preferred halogens are fluorine and chlorine.

When used herein, the term “aryl” as a group or part of a group means a carbocyclic aromatic ring. Examples of suitable aryl groups include phenyl and naphthyl.

When used herein, the term “heteroaryl” as a group or part of a group means a 5- to 10-membered heteroaromatic ring system containing 1 to 4 heteroatoms selected from N, O and S. Particular examples of such groups include pyrrolyl, furanyl, thienyl, pyridyl, pyrazolyl, imidazolyl, oxazolyl, isoxazolyl, thiazolyl, isothiazolyl, pyrazinyl, pyrimidinyl, pyridazinyl, triazolyl, oxadiazolyl, thiadiazolyl, triazinyl, tetrazolyl, indolyl, benzothienyl and quinolinyl.

Where a compound or group is described as “optionally substituted” one or more substituents may be present. Optional substituents are not particularly limited and may, for instance, be selected from C₁₋₆ alkyl, C₂₋₆ alkenyl, C₃₋₇ cycloalkyl, C₃₋₇ heterocycloalkyl, aryl, aryl(C₁₋₆)alkyl, heteroaryl, heteroaryl(C₁₋₆)alkyl, C₁₋₆ alkoxy, aryloxy, aryl(C₁₋₆)alkoxy, heteroaryloxy, heteroaryl(C₁₋₆)alkoxy, amino, nitro, halo, hydroxy, carboxy, formyl, cyano and trihalomethyl groups. Furthermore, optional substituents may be attached to the compounds or groups which they substitute in a variety of ways, either directly or through a connecting group of which the following are examples: amine, amide, ester, ether, thioether, sulfonamide, sulfamide, sulfoxide, urea, thiourea and urethane. As appropriate an optional substituent may itself be substituted by another substituent, the latter being connected directly to the former or through a connecting group such as those exemplified above.

Specific compounds within the scope of this invention include:
3-cyclohexyl-1-[2-(dimethylamino)-2-oxoethyl]-2-(4-methylphenyl)-1H-indole-6-carboxylic acid,
3-cyclohexyl-1-[2-(dimethylamino)-2-oxoethyl]-2-(2-fluorophenyl)-1H-indole-6-carboxylic acid,
3-cyclohexyl-1-[2-(dimethylamino)-2-oxoethyl]-2-(3-methylphenyl)-1H-indole-6-carboxylic acid,
3-cyclohexyl-1-[2-(dimethylamino)-2-oxoethyl]-2-(2-hydroxypyrimidin-5-yl)-1H-indole-6-carboxylic acid,
3-cyclohexyl-1-[2-(dimethylamino)-2-oxoethyl]-2-(3-furyl)-1H-indole-6-carboxylic acid,
3-{6-carboxy-3-cyclohexyl-1-[2-(dimethylamino)-2-oxoethyl]-1H-indol-2-yl}pyridinium trifluoroacetate,
3-cyclohexyl-1-[2-(dimethylamino)-2-oxoethyl]-2-phenyl-1H-indole-6-carboxylic acid,
3-cyclohexyl-1-[2-(methylamino)-2-oxoethyl]-2-phenyl-1H-indole-6-carboxylic acid,
3-cyclohexyl-1-(2-morpholin-4-yl-2-oxoethyl)-2-phenyl-1H-indole-6-carboxylic acid,
3-cyclohexyl-1-(2-{[(1-methylpyrrolidin-3-yl)methyl]amino}-2-oxoethyl)-2-phenyl-1H-indole-6-carboxylic acid hydrochloride,
3-cyclohexyl-1-[2-(4-methylpiperazin-1-yl)-2-oxoethyl]-2-phenyl-1H-indole-6-carboxylic acid trifluoroacetate,
3-cyclohexyl-1-(2-{[1-(5-methyl-4H-1,2,4-triazol-3-yl)ethyl]amino}-2-oxoethyl)-2-phenyl-1H-indole-6-carboxylic acid trifluoroacetate,
3-cyclohexyl-1-(2-{methyl[(1-methylpiperidin-3-yl)methyl]amino}-2-oxoethyl)-2-phenyl-1H-indole-6-carboxylic acid trifluoroacetate,
3-cyclohexyl-1-(2-{[(1-methylpiperidin-3-yl)methyl]amino}-2-oxoethyl)-2-phenyl-1H-indole-6-carboxylic acid trifluoroacetate,
3-cyclohexyl-1-(2-{methyl[(1-methylpiperidin-2-yl)methyl]amino}-2-oxoethyl)-2-phenyl-1H-indole-6-carboxylic acid trifluoroacetate,
3-cyclohexyl-1-(2-{methyl[(5-methyl-1H-imidazol-2-yl)methyl]amino}-2-oxoethyl)-2-phenyl-1H-indole-6-carboxylic acid trifluoroacetate,
3-cyclohexyl-1-(2-{[2-(dimethylamino)ethyl]amino}-2-oxoethyl)-2-phenyl-1H-indole-6-carboxylic acid trifluoroacetate,
3-cyclohexyl-1-(2-{[2-(1-methylpyrrolidin-3-yl)ethyl]amino}-2-oxoethyl)-2-phenyl-1H-indole-6-carboxylic acid trifluoroacetate,
2-[3-cyclohexyl-2-phenyl-6-(1H-tetrazol-5-yl)-1H-indol-1-yl]-N,N-dimethylacetamide,
3-cyclohexyl-N-methyl-1-(2-morpholin-4-yl-2-oxoethyl)-2-phenyl-1H-indole-6-carboxamide,
3-cyclohexyl-1-[2-(dimethylamino)-2-oxoethyl]-2-phenyl-1H-pyrrolo[2,3-b]pyridine-6-carboxylic acid,
3-cyclohexyl-1-[2-(4-methylpiperazin-1-yl)-2-oxoethyl]-2-phenyl-1H-pyrrolo[2,3-b]pyridine-5-carboxylic acid,
3-cyclohexyl-2-{3-[2-(dimethylamino)ethyl]phenyl}-1-[2-(dimethylamino)-2-oxoethyl]-1H-indole-6-carboxylic acid,
3-cyclohexyl-1-[2-(dimethylamino)prop-2-en-1-yl]-2-(2-methyl-1,2,3,4-tetrahydroisoquinolin-7-yl)-1H-indole-6-carboxylic acid,
2-[3-cyclohexyl-6-(5-oxo-4,5-dihydro-1,2,4-oxadiazol-3-yl)-2-phenyl-1H-indol-1-yl]-N,N-dimethylacetamide,
3-[3-cyclohexyl-1-{2-[4-(dimethylamino)piperidin-1-yl]-2-oxoethyl}-2-(3-furyl)-1H-indol-6-yl]-1,2,4-oxadiazol-5(4H)-one,
3-cyclohexyl-1-[2-(dimethylamino)-2-oxoethyl]-N-(ethylsulfonyl)-2-phenyl-1H-indole-6-carboxamide,
N-(benzylsulfonyl)-3-cyclohexyl-1-[2-(dimethylamino)-2-oxoethyl]-2-phenyl-1H-indole-6-carboxamide,
2-(4-chlorophenyl)-3-cyclohexyl-1-(2-morpholin-4-yl-2-oxoethyl)-1H-indole-6-carboxylic acid,
3-cyclohexyl-2-(4-methoxyphenyl)-1-(2-morpholin-4-yl-2-oxoethyl)-1H-indole-6-carboxylic acid,
1-{[5-carboxy-3-cyclohexyl-2-(4-methoxyphenyl)-1H-indol-1-yl]acetyl}-N,N-dimethylpiperidin-4-aminium trifluoroacetate,
1-{[5-carboxy-3-cyclohexyl-2-(3-furyl)-1H-indol-1-yl]acetyl}-N,N-dimethylpiperidin-4-aminium trifluoroacetate,
(4-{[6carboxy-2-(4-chlorophenyl)-3-cyclohexyl-1H-indol-1-yl]acetyl}morpholin-2-yl)-N,N-dimethylmethanaminium trifluoroacetate;
and pharmaceutically acceptable salts thereof.

Further compounds within the scope of this invention include:
1-{2-[benzyl(methyl)amino]-2-oxoethyl}-3-cyclohexyl-2-phenyl-1H-indole-6-carboxylic acid,
1-(2-amino-2-oxoethyl)-3-cyclohexyl-2-phenyl-1H-indole-6-carboxylic acid,
3-cyclohexyl-1-[2-(7-methyl-2,7-diazaspiro[4.4]non-2-yl)-2-oxoethyl]-2-phenyl-1H-indole-6-carboxylic acid,
1-[2-(benzylamino)-2-oxoethyl]-3-cyclohexyl-2-phenyl-1H-indole-6-carboxylic acid,
3-cyclohexyl-1-(2-{[(3R,4R)-4hydroxy-1,1-dioxidotetrahydro-3-thienyl]amino}-2-oxoethyl)-2-phenyl-1H-indole-6-carboxylic acid,
3-cyclohexyl-1-[2-oxo-2-(3-pyridin-3-ylpyrrolidin-1-yl)ethyl]-2-phenyl-1H-indole-6-carboxylic acid,
3-cyclohexyl-1-(2-{methyl[1-(1,3-thiazol-2-yl)ethyl]amino}-2-oxoethyl)-2-phenyl-1H-indole-6-carboxylic acid,
3-cyclohexyl-1-(2-[4-(4-methyl-4H-1,2,4-triazol-3-yl)piperidin-1-yl]-2-oxoethyl}-2-phenyl-1H-indole-6-carboxylic acid
3-cyclohexyl-1-{2-[4-(6-methoxypyridin-2-yl)piperazin-1-yl]-2-oxoethyl}-2-phenyl-1H-indole-6-carboxylic acid,
3-cyclohexyl-1-[2-(dimethylamino)-2-oxoethyl]-2-pyridin-4-yl-1H-indole-6-carboxylic acid,
3-cyclohexyl-1-(2-[3-[(dimethylamino)methyl]piperidin-1-yl}-2-oxoethyl)-2-phenyl-1H-indole-6-carboxylic acid,
3-cyclohexyl-1-(2-{2-[2-(dimethylamino)ethyl]piperidin-1-yl}-2-oxoethyl)-2-phenyl-1H-indole-6-carboxylic acid,
(1-pyridin-4-ylethyl)amino]-2-oxoethyl}-2-phenyl-1H-indole-6-carboxylic acid,
3-cyclohexyl-1-(2-oxo-2-{[(1-piperidin-1-ylcyclopentyl)methyl]amino}ethyl)-2-phenyl-1H-indole-6-carboxylic acid,
3-cyclohexyl-1-[2-oxo-2-(2-pyridin-4-ylpyrrolidin-1-yl)ethyl]-2-phenyl-1H-indole-6-carboxylic acid,
3-cyclohexyl-1-(2-[(1S,4S)-5-methyl-2,5-diazabicyclo[2.2.1]hept-2-yl]-2-oxoethyl]2-phenyl-1H-indole-6-carboxylic acid,
3-cyclohexyl-1-[2-(hexahydropyrrolo[1,2-a]pyrazin-2(1H)-yl)-2-oxoethyl]-2-phenyl-1H-indole-6-carboxylic acid,
3-cyclohexyl-1-(2-{[2-(4-methylpiperazin-1-yl)ethyl]amino}-2-oxoethyl)-2-phenyl-1H-indole-6-carboxylic acid,
3-cyclohexyl-1-{2-[(cyclopropylmethyl)amino]-2-oxoethyl]-2-phenyl-1H-indole-6-carboxylic acid,
3-cyclohexyl-1-[2-oxo-2-(prop-2-yl-1-ylamino)ethyl]-2-phenyl-1H-indole-6-carboxylic acid,
3-cyclohexyl-1-{2-[(2-morpholin-4-ylethyl)amino]-2-oxoethyl}-2-phenyl-1H-indole-6-carboxylic acid,
3-cyclohexyl-1-{2-[methyl(1-methylpiperidin-4-yl)amino]-2-oxoethyl}-2-phenyl-1H-indole-6-carboxylic acid,
3-cyclohexyl-1-(2-{[-2-(diisopropylamino)ethyl]amino)-2-oxoethyl)-2-phenyl-1H-indole-6-carboxylic acid,
3-cyclohexyl-1-[2-(dimethylamino)-2-oxoethyl]-2-(3-fluoro-4-hydroxyphenyl)-1H-indole-6-carboxylic acid,
3-cyclohexyl-1-[2-(dimethylamino)-2-oxoethyl]-2-(4-hydroxyphenyl)-1H-indole-6-carboxylic acid,
2-(3-chlorophenyl)-3-cyclohexyl-1-[2-(dimethylamino)-2-oxoethyl]-1H-indole-6-carboxylic acid,
2-(4-chlorophenyl)-3-cyclohexyl-1-[2-(dimethylamino)-2-oxoethyl]-1H-indole-6-carboxylic acid,
3-cyclohexyl-1-[2-(dimethylamino)-2-oxoethyl]-2-(3-fluorophenyl)-1H-indole-6-carboxylic acid,
3-cyclohexyl-1-[2-(dimethylamino)-2-oxoethyl]-2-(4-fluorophenyl)-1H-indole-6-carboxylic acid,
3-cyclohexyl-1-[2-(dimethylamino)-2-oxoethyl]-2-(3-thienyl)-1H-indole-carboxylic acid,
2-[4(aminocarbonyl)phenyl]-3-cyclohexyl-1-[2-(dimethylamino)-2-oxoethyl]-1H-indole-6-carboxylic acid,
2-[3-(acetylamino)phenyl]-3-cyclohexyl-1-[2-(dimethylamino)-2-oxoethyl]-1H-indole-6-carboxylic acid,
3-cyclohexyl-1-[2-(dimethylamino)-2-oxoethyl]-2-[3-(1H-pyrazol-1-yl)phenyl]-1H-indole-6-carboxylic acid,
3-cyclohexyl-1-[2-(dimethylamino)-2-oxoethyl]-2-(3-hydroxyphenyl)-1H-indole-6-carboxylic acid,
3-cyclohexyl-1-[2-(dimethylamino)-2-oxoethyl]-2-(2-methylphenyl)-1H-indole-6-carboxylic acid,
3-cyclohexyl-2-(3,5-difluorophenyl)-1-[2-(dimethylamino)-2-oxoethyl]-1H-indole-6-carboxylic acid,
3-cyclohexyl-2-(3,4-difluorophenyl)-1-[2-(dimethylamino)-2-oxoethyl]-1H-indole-6-carboxylic acid,
3-cyclohexyl-2-(2,4-difluorophenyl)-1-[2-(dimethylamino)-2-oxoethyl]-1H-indole-6-carboxylic acid,
3-cyclohexyl-1-[2-(dimethylamino)-2-oxoethyl]-2-(4-methoxyphenyl)-1H-indole-6-carboxylic acid,
3-cyclohexyl-1-[2-(dimethylamino)-2-oxoethyl]-2-(3-methoxyphenyl)-1H-indole-6-carboxylic acid,
3-cyclohexyl-1-[2-(dimethylamino)-2-oxoethyl]-2-(2-methoxyphenyl)-1H-indole-6-carboxylic acid,
3-cyclohexyl-1-[2-(dimethylamino)-2-oxoethyl]-2-(2-hydroxyphenyl)-1H-indole-6-carboxylic acid,
2-(2-chlorophenyl)-3-cyclohexyl-1-[2-(dimethylamino)-2-oxoethyl]-1H-indole-6-carboxylic acid,
3-cyclohexyl-2-(3-fluorophenyl)-1-(2-{methyl[(1-methylpiperidin-3-yl)methyl]amino}-2-oxoethyl)-1H-indole-6-carboxylic acid,
3-cyclohexyl-1-(2-{3-[(dimethylamino)methyl]piperidin-1-yl}-2-oxoethyl)-2-(3-fluorophenyl)-1H-indole-6-carboxylic acid;
and pharmaceutically acceptable salts thereof.

Specific compounds within the scope of this invention also include:
3-cyclopentyl-1-{2-[methyl(phenyl)amino]-2-oxoethyl}-2-phenyl-1H-indole-6-carboxylic acid,
3-cyclopentyl-1-[2-oxo-2-(4-pyrrolidin-1-ylpiperidin-1-yl)ethyl]-2-phenyl-1H-indole-6-carboxylic acid,
3-cyclohexyl-1-[2-oxo-2-(4-pyrrolidin-1-ylpiperidin-1-yl)ethyl]-2-pyridin-4-yl-1H-indole-6-carboxylic acid,
1-(2-{[(1-acetylpyrrolidin-2-yl)methyl]amino}-2-oxoethyl)-3-cyclohexyl-2-pyridin-4-yl-1H-indole-6-carboxylic acid,
3-cyclohexyl-1-{2-[3-(dimethylamino)piperidin-1-yl]-2-oxoethyl}-2-pyridin-3-yl-1H-indole-6-carboxylic acid,
3-cyclohexyl-1-{2-[[2-(dimethylamino)-2-oxyethyl](methyl)amino]-2-oxoethyl}-2-pyridin-3-yl-1H-indole-6-carboxylic acid,
3-cyclohexyl-1-[2-(hexahydropyrrolo[1,2-a]pyrazin-2(1H)-yl)-2-oxoethyl]-2-pyridin-3-yl-1H-indole-6-carboxylic acid,
3-cyclopentyl-1-(2-{methyl[(1-methylpiperidin-4-yl)methyl]amino}-2-oxoethyl)-2-phenyl-1H-indole-6-carboxylic acid,
3-cyclopentyl-1-(2-{[(1-ethyl-5-oxopyrrolidin-3-yl)methyl]amino}-2-oxoethyl)-2-phenyl-1H-indole-6-carboxylic acid,
3-cyclohexyl-2-(4-[2-(dimethylamino)-2-oxoethoxy]phenyl}-1-{2-[methyl(pyrazin-2-ylmethyl)amino]-2-oxoethyl}-1H-indole-6-carboxylic acid,
2-(4-chloro-2-fluorophenyl)-3-cyclohexyl-1-(2-morpholin-4-yl-2-oxoethyl)-1H-indole-6-carboxylic acid,
3-cyclohexyl-1-(2-{[(1,1-dioxidotetrahydro-3-thienyl)methyl]amino}-2-oxoethyl)-2-(3-fluorophenyl)-1H-indole-6-carboxylic acid,
2-biphenyl-3-yl-3-cyclohexyl-1-{2-[4-(dimethylamino)piperidin-1-yl]-2-oxoethyl)-1H-indole-6-carboxylic acid,
2-(2-chlorophenyl)-3-cyclohexyl-1-{2-[4-(dimethylamino)piperidin-1-yl]-2-oxoethyl}-1H-indole-6-carboxylic acid,
3-cyclohexyl-1-(2-[4-(dimethylamino)piperidin-1-yl]-2-oxoethyl}-2-(5-fluoro-2-methoxyphenyl)-1H-indole-6-carboxylic acid,
3-cyclohexyl-1-{2-[4(dimethylamino)piperidin-1-yl]-2-oxoethyl}-2-(3-thienyl)-1H-indole-6-carboxylic acid,
2-[4-(benzyloxy)phenyl]-3-cyclohexyl-1-{2-[4-(dimethylamino)piperidin-1-yl]-2-oxoethyl}-1H-indole-6-carboxylic acid,
3-cyclohexyl-1-{2-[4-(dimethylamino)piperidin-1-yl]-2-oxoethyl}-2-(4-isopropoxyphenyl)-1H-indole-6-carboxylic acid,
3-cyclohexyl-1-{2-[4-(dimethylamino)piperidin-1-yl]-2-oxoethyl}-2-[3-(piperidin-1-ylcarbonyl)phenyl]-1H-indole-6-carboxylic acid,
3-cyclohexyl-1-{2-[4-(dimethylamino)piperidin-1-yl]-2-oxoethyl}-2-(3-methylphenyl)-1H-indole-6-carboxylic acid,
3-cyclohexyl-1-[2-(methylamino)-2-oxoethyl]-2-phenyl-1H-indole-5-carboxylic acid,
3-cyclohexyl-1-(2-{methyl[(1-methylpiperidin-3-yl)methyl]amino}-2-oxoethyl)-2-phenyl-1H-indole-5-carboxylic acid,
3-cyclohexyl-1-{2-[[2-(dimethylamino)-2-oxoethyl](methyl)amino]-2-oxyethyl}-2-phenyl-1H-indole-5-carboxylic acid,
1-[2-(2-{[acetyl(methyl)amino]methyl}morpholin-4-yl)-2-oxoethyl]-3-cyclohexyl-2-(3-fluorophenyl)-1H-indole-6-carboxylic acid,
3-cyclopentyl-1-[2-(1,1-dioxidothiomorpholin-4-yl)-2-oxoethyl]-2-phenyl-1H-indole-5-carboxylic acid,
3-cyclopentyl-1-[2-oxo-2-(4-pyrrolidin-1-ylpiperidin-1-yl)ethyl]-2-phenyl-1H-indole-5-carboxylic acid,
3-cyclopentyl-1-{2-[(cyclopropylmethyl)amino]-2-oxoethyl}-2-phenyl-1H-indole-5-carboxylic acid,
3-cyclopentyl-1-(2-{[(1-ethyl-5-oxopyrrolidin-3-yl)methyl]amino}-2-oxoethyl)-2-phenyl-1H-indole-5-carboxylic acid,
3-cyclohexyl-1-{2-[4-(dimethylamino)piperidin-1-yl]-2-oxoethyl}-2-pyrimidin-5-yl-1H-indole-6-carboxylic acid,
2-(4-chlorophenyl)-3-cyclohexyl-1-[2-(4-methyl-1,4-diazepan-1-yl)-2-oxoethyl]-1H-indole-6-carboxylic acid,
2-(4-chlorophenyl)-3-cyclohexyl-1-[2-(4-isopropylpiperazin-1-yl)-2-oxoethyl]-1H-indole-6-carboxylic acid,
2-(4-chlorophenyl)-3-cyclohexyl-1-[2-oxo-2-(3-pyrrolidin-1-ylpiperidin-1-yl)ethyl]-1H-indole-6-carboxylic acid,
2-(4-chlorophenyl)-3-cyclohexyl-1-(2-oxo-2-piperazin-1-ylethyl)-1H-indole-6-carboxylic acid,
3-cyclohexyl-2-(3-furyl)-1-[2-oxo-2-(4-pyrrolidin-1-ylpiperidin-1-yl)ethyl]-1H-indole-6-carboxylic acid,
3-cyclohexyl-1-(2-{2-[(dimethylamino)methyl]morpholinyl-4-yl}-2-oxoethyl)-2-(3-furyl)-1H-indole-6-carboxylic acid,
1-[2-(4-azetidin-1-ylpiperidin-1-yl)-2-oxoethyl]-3-cyclohexyl-2-(4methoxyphenyl)-1H-indole-6-carboxylic acid,
3-cyclohexyl-2-(4-methoxyphenyl)-1-[2-oxo-2-(4-pyrrolidin-1-ylpiperidin-1-yl)ethyl]-1H-indole-6-carboxylic acid,
3-cyclohexyl-1-{2-[4-(diethylamino)piperidin-1-yl]-2-oxoethyl}-2-(4-methoxyphenyl)-1H-indole-6-carboxylic acid,
3-cyclohexyl-2-{3-[(dimethylamino)methyl]phenyl}-1-[2-(dimethylamino)- 2-oxoethyl]-1H-indole-6-carboxylic acid,
3-cyclohexyl-1-[2-(dimethylamino)-2-oxoethyl]-2-{3-[(1-methylpiperidin-4yl)oxy]phenyl}-H-indole-6-carboxylic acid,
3-cyclohexyl-1-[2-(dimethylamino)-2-oxoethyl]-2-phenyl-1H-pyrrolo[3,2-b]pyridine-6-carboxylic acid,
3-cyclohexyl-1-[2-(dimethylamino)-2-oxoethyl]-2-(1-naphthyl)-1H-indole-6-carboxylic acid,
3-cyclohexyl-1-[2-(dimethylamino)-2-oxoethyl]-2-(2-naphthyl)-1H-indole-6-carboxylic acid,
3-cyclohexyl-1-(2-morpholin-4-yl-2-oxoethyl)-1H,1′H-2,5′-bisindole-6-carboxylic acid,
3-cyclohexyl-1-[2-(dimethylamino)-2-oxoethyl]-2-(8-methylquinolin-4-yl)-1H-indole-6-carboxylic acid;
and pharmaceutically acceptable salts thereof.

Further compounds within the scope of this invention also include:
3-cyclohexyl-N-methyl-1-[2-(4-methylpiperazin-1-yl)-2-oxoethyl]-2-phenyl-1H-indole-6-carboxamide,
3-cyclohexyl-N-[(4-methyl-1H-imidazol-2-yl)methyl]-1-(2-morpholin-4-yl-2-oxoethyl)-2-phenyl-1H-indole-6-carboxamide,
3-cyclohexyl-1-(2-morpholinyl-2-oxoethyl)-2-phenyl-1H-indole-6-carboxamide,
3-cyclohexyl-N,N-dimethyl-1-(2-morpholinyl-2-oxoethyl)-2-phenyl-1H-indole-6-carboxamide,
3-cyclohexyl-N-isopropyl-1-(2-morpholinyl-2-oxoethyl)-2-phenyl-1H-indole-6-carboxamide,
N-allyl-3-cyclohexyl-1-(2-morpholin-4yl-2-oxoethyl)-2-phenyl-1H-indole-6-carboxamide,
3-cyclohexyl-N-[2-(dimethylamino)ethyl]-1-(2-morpholinyl-2-oxoethyl)-2-phenyl-1H-indole-6-carboxamide,
3-cyclohexyl-N-[(1-methylpiperidin-3-yl)methyl]-1-(2-morpholinyl-2-oxoethyl)-2-phenyl-1-1H -indole-6-carboxamide,
3-cyclohexyl-N-[(1-methylpyrrolidin-3-yl)methyl]-1-(2-morpholin-4-yl-2-oxoethyl)-2-phenyl-1H-indole-6-carboxamide,
3-cyclohexyl-6-[(4-methylpiperazin-1-yl)carbonyl]-1-(2-morpholin-4-yl-2-oxoethyl)-2-phenyl-1H-indole,
3-cyclohexyl-1-(2-morpholinyl-2-oxoethyl)-2-phenyl-N-(tetrahydrofuran-3-yl)-1H-indole-6-carboxamide,
3-cyclohexyl-N-(1,1-dioxidotetrahydro-3-thienyl)-1-(2-morpholin-4-yl-2-oxoethyl)-2-phenyl-1H-indole-6-carboxamide,
3-cyclohexyl-N-(2-furylmethyl)-1-(2-morpholin-4-yl-2-oxoethyl)-2-phenyl-1H-indole-6-carboxamide,
3-cyclohexyl-N-[(6-methylpyridin-2-yl)methyl]-1-(2-morpholin-4yl-2-oxoethyl)-2-phenyl-1H-indole-6-carboxamide,
3-cyclohexyl-1-(2-morpholin-4-yl-2-oxoethyl)-N,2-diphenyl-1H-indole-6-carboxamide,
N-benzyl-3-cyclohexyl-1-(2-morpholin-4-yl-2-oxoethyl)-2-phenyl-1H-indole-6-carboxamide,
4-{[3-cyclohexyl-1-(2-morpholin-4-yl-2-oxoethyl)-2-phenyl-1H-indol-6-yl]carbonyl}piperazin-2-one,
3-cyclohexyl-N-(2-methoxyethyl)-1-(2-morpholin-4-yl-2-oxoethyl)-2-phenyl-1H-indole-6-carboxamide,
3-cyclohexyl-N-(2-morpholin-4-ylethyl)-1-(2-morpholin-4-yl-2-oxoethyl)-2-phenyl-1H-indole-6-carboxamide,
3-cyclohexyl-N-[2-(1-methylpyrrolidin-3-yl)ethyl]-1-(2-morpholin-4-yl-2-oxoethyl)-2-phenyl-1H-indole-6-carboxamide,
N-{[3-cyclohexyl-1-(2-morpholin-4-yl-2-oxoethyl)-2-phenyl-1H-indol-6-yl]carbonyl}-5-hydroxy-L-tryptophan,
3-cyclohexyl-1-(2-morpholin-4-yl-2-oxoethyl)-2-phenyl-N-[2-(1H-pyrazol-1-yl)ethyl]-1H-indole-6-carboxamide;
and pharmaceutically acceptable salts thereof.

Specific compounds within the scope of this invention also include:
3-{3-cyclohexyl-1-[2-(4-methylpiperazin-1-yl)-2-oxoethyl]-2-phenyl-1H-indol-6-yl)-1,2,4-oxadiazol-5(4H)-one,
2-l3-cyclohexyl-6-(5-oxo-4,5-dihydro-1,2,4-oxadiazol-3-yl)-2-phenyl-1H-indol-1-yl]-N-methyl-N-[(1-methylpiperidin-3-yl)methyl]acetamide,
2-[3-cyclohexyl-6-(5-oxo-4,5-dihydro-1,2,4-oxadiazol-3-yl)-2-phenyl-1H-indol-1-yl]-N,N-dimethylacetamide,
3-[3-cyclohexyl-1-(2-morpholin-4-yl-2-oxoethyl)-2-phenyl-1H-indol-6-yl]-1,2,4-oxadiazol-5(4H)-one,
2-[3-cyclohexyl-6-(5-oxo-4,5-dihydro-1,2,4-oxadiazol-3-yl)-2-phenyl-1H-indol-1-yl]-N-[(1-methylpyrrolidin-3-yl)methyl]acetamide,
3-[3-cyclohexyl-1-{2-[4-(dimethylamino)piperidin-1-yl]-2-oxoethyl}-2-(2-methylphenyl)-1H-indol-6-yl]-1,2,4-oxadiazol-5(4H)-one,
3-[3-cyclohexyl-1-{2-l4-(dimethylamino)piperidin-1-yl]-2-oxoethyl}-2-(2-fluorophenyl)-1H-indol-6-yl]-1,2,4oxadiazol-5 (4H)-one,
2-[3-cyclohexyl-2-(3-methoxyphenyl)-6-(5-oxo-4,5-dihydro-1,2,4-oxadiazol-3-yl)-1H-indol-1-yl]-N,N-dimethylacetamide,
3-[3-cyclohexyl-1-{2-[4-(dimethylamino)piperidin-1-yl]-2-oxoethyl}-2-(3-methoxyphenyl)-1H-indol-6-yl]-1,2,4-oxadiazol-5(4H)-one,
2-[3-cyclohexyl-6-(5-oxo-4,5-dihydro-1,2,4-oxadiazol-3-yl)-2-[3-(piperidin-1-ylmethyl)phenyl]-1H-indol-1-yl }-N,N-dimethylacetamide,
3-{3-cyclohexyl-1-(2-{3-[(dimethylamino)methyl]piperidin-1-yl}-2-oxoethyl)-2-[3-(piperidin-1-ylmethyl)phenyl]-1H-indol-6-yl}-1,2,4-oxadiazol-5(4H)-one,
3-[3-cyclohexyl-1-[2-(dimethylamino)-2-oxoethyl]-6-(5-oxo-4,5-dihydro-1,2,4-oxadiazol-3-yl)-1H-indol-2-yl]-N,N-dimethylbenzamide,
2-[3-cyclohexyl-2-(4-methoxyphenyl)-6-(5-oxo-4,5-dihydro-1,2,4-oxadiazol-3-yl)-1H-indol-1-yl]-N,N-dimethylacetamide,
2-[2-(4-chlorophenyl)-3-cyclohexyl-6-(5-oxo-4,5-dihydro-1,2,4oxadiazol-3-yl)-1H-indol-1-yl]-N,N-dimethylacetamide,
3-(2-(4-chlorophenyl)-3-cyclohexyl-1-{2-[4-(dimethylamino)piperidin-1-yl]-2-oxoethyl}-1H-indol-6-yl)-1,2,4-oxadiazol-5(4H)-one,
3-[3-cyclohexyl-1-(2-{2-[(dimethylamino)methyl]morpholin-4-yl}-2-oxoethyl)-2-(4-fluorophenyl)-1H-indol-6-yl]-1,2,4-oxadiazol-5(4H)-one,
2-[3-cyclohexyl-2-(3-furyl)-6-(5-oxo-4,5-dihydro-1,2,4-oxadiazol-3-yl)-1H-indol-1-yl]-N,N-dimethylacetamide,
3-[3-cyclohexyl-1-(2-{2-[(dimethylamino)methyl]morpholin-4-yl}-2-oxoethyl)-2-(3-furyl)-1H-indol-6-yl]-1,2,4-oxadiazol-5(4H)-one,
3-[3-cyclohexyl-1-{2-[4-(dimethylamino)piperidin-1-yl]-2-oxoethyl)-2-(3-furyl)-1H-indol-6yl]-1,2,4oxadiazol-5 (4H)-one,
3-[3-cyclohexyl-1-(2-{2-[(dimethylamino)methyl]morpholin-4-yl}-2-oxoethyl)-2-(5-methyl-2-furyl)-1H-indol-6-yl]-1,2,4-oxadiazol-5(4H)-one,
3-{3-cyclohexyl-1-{2-[4-(dimethylamino)piperidin-1-yl]-2-oxoethyl)-2-[5-(piperidin-1-ylmethyl)-2-furyl]-1H-indol-6-yl )-1,2,4-oxadiazol-5(4H)-one,
2-[3-cyclohexyl-2-(1-methyl-1H-pyrazol-4-yl)-6-(5-oxo-4,5-dihydro-1,2,4-oxadiazol-3-yl)-1H-indol-1-yl]-N,N-dimethylacetamide,
2-[3-cyclohexyl-6-(5-oxo-4,5-dihydro-1,2,4-oxadiazol-3-yl)-2-pyridin-3-yl-1H-indol-1-yl]-N,N-dimethylacetamide,
3-[3-cyclohexyl-1-(2-{3-[(dimethylamino)methyl]piperidin-1-yl}-2-oxoethyl)-2-pyridin-3-yl-1H-indol-6-yl]-1,2,4-oxadiazol-5(4H)-one,
2-[3-cyclohexyl-2-(6-methoxypyridin-3-yl)-6-(5-oxo-4,5-dihydro-1,2,4-oxadiazol-3-yl)-1H-indol-1-yl]-N,N-dimethylacetamide,
3-[3-cyclohexyl-1-{2-[4-(dimethylamino)piperidin-1-yl]-2-oxoethyl)-2-(6-methoxypyridin-3-yl)-1H-indol-6-yl]-1,2,4-oxadiazol-5(4H)-one,
2-[3-cyclohexyl-2-(2-methoxypyridin-4-yl)-6-(5-oxo-4,5-dihydro-1,2,4-oxadiazol-3-yl)-1H-indol-1-yl]-N,N-dimethylacetamide,
2-[3-cyclohexyl-2-{2-[2-(dimethylamino)ethoxy]pyridin4-yl]-6-(5-oxo-4,5-dihydro-1,2,4-oxadiazol-3-yl)-1H-indol-1-yl]-N,N-dimethylacetamide,
3-cyclohexyl-1-[2-(dimethylamino)-2-oxoethyl]-N-(methylsulfonyl)-2-phenyl-1H-indole-6-carboxamide,
3-cyclohexyl-1-[2-(dimethylamino)-2-oxoethyl]-N-(ethylsulfonyl)-2-phenyl-1H-indole-6-carboxamide,
3-cyclohexyl-1-[2-(dimethylamino)-2-oxoethyl]-N-(ethylsulfonyl)-2-(3-furyl)-1H-indole-6-carboxamide,
3-cyclohexyl-1-[2-(dimethylamino)-2-oxoethyl]-N-(ethylsulfonyl)-2-(6-methoxypyridin-3-yl)-1H-indole-6-carboxamide,
3-cyclohexyl-N-(ethylsulfonyl)-2-(4-methoxyphenyl)-1-(2-morpholin-4yl-2-oxoethyl)-1H-indole-6-carboxamide,
3-cyclohexyl-1-[2-(dimethylamino)-2-oxoethyl]-N-(isopropylsulfonyl)-2-phenyl-1H-indole-6-carboxamide,
3-cyclohexyl-1-[2-(dimethylamino)-2-oxoethyl]-2-phenyl-N-(propylsulfonyl)-1H-indole-6-carboxamide,
3-cyclohexyl-1-[2-(dimethylamino)-2-oxoethyl]-2-phenyl-N-[(2,2,2-trifluoroethyl)sulfonyl]-1H-indole-6-carboxamide,
benzyl (2-{[({3-cyclohexyl-1-[2-(dimethylamino)-2-oxoethyl]-2-phenyl-1H-indol-6-yl}carbonyl)amino]sulfonylethyl)carbamate,
N-[(2-aminoethyl)sulfonyl]-3-cyclohexyl-1-[2-(dimethylamino)-2-oxoethyl]-2-phenyl-1H-indole-6-carboxamide,
3-cyclohexyl-N-{[2-(dimethylamino)ethyl]sulfonyl}-1-[2-(dimethylamino)-2-oxoethyl]-2-phenyl-1H-indole-6-carboxamide,
3-cyclohexyl-1-[2-(dimethylamino)-2-oxoethyl]-2-phenyl-N-[(2-phenylethyl)sulfonyl]-1H-indole-6-carboxamide,
N-(benzylsulfonyl)-3-cyclohexyl-1-[2-(dimethylamino)-2-oxoethyl]-2-phenyl-1H-indole-6-carboxamide,
N-(benzylsulfonyl)-3-cyclohexyl-1-(2-{methyl[(1-methylpiperidin-3-yl)methyl]amino}-2-oxoethyl)-2-phenyl-1H-indole-6-carboxamide,
N-(benzylsulfonyl)-3-cyclohexyl-1-[2-(dimethylamino)-2-oxoethyl]-2-(3-furyl)-1H-indole-6-carboxamide,
3-cyclohexyl-1-[2-(dimethylamino)-2-oxoethyl]-2-phenyl-N-(phenylsulfonyl)-1H-indole-6-carboxamide,
3-cyclohexyl-1-[2-(dimethylamino)-2-oxoethyl]-N-[(4-methoxyphenyl)sulfonyl]-2-phenyl-1H-indole-6-carboxamide,
3-cyclohexyl-1-[2-(dimethylamino)-2-oxoethyl]-2-phenyl-N-(pyridin-3-ylsulfonyl)-1H-indole-6-carboxamide,
3-cyclohexyl-1-[2-(dimethylamino)-2-oxoethyl]-2-phenyl-N-(3-thienylsulfonyl)-1H-indole-6-carboxamide;
and pharmaceutically acceptable salts thereof.

For use in medicine, the salts of the compounds of formula (I) will be non-toxic pharmaceutically acceptable salts. Other salts may, however, be useful in the preparation of the compounds according to the invention or of their non-toxic pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, fumaric acid, p-toluenesulfonic acid, maleic acid, succinic acid, acetic acid, citric acid, tartaric acid, carbonic acid, phosphoric acid or sulfuric acid. Salts of amine groups may also comprise quaternary ammonium salts in which the amino nitrogen atom carries a suitable organic group such as an alkyl, alkenyl, alkynyl or alkyl moiety. Furthermore, where the compounds of the invention carry an acidic moiety, suitable pharmaceutically acceptable salts thereof may include metal salts such as alkali metal salts, e.g. sodium or potassium salts; and alkaline earth metal salts, e.g. calcium or magnesium salts.

The salts may be formed by conventional means, such as by reacting the free base form of the product with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention includes within its scope prodrugs of the compounds of formula (I) above. In general, such prodrugs will be functional derivatives of the compounds of formula (I) which are readily convertible in vivo into the required compound of formula (I). Conventional procedures for the selection and preparation of suitable prodrug derivatives are described, for example, in “Design of Prodrugs”, ed. H. Bundgaard, Elsevier, 1985.

A prodrug may be a pharmacologically inactive derivative of a biologically active substance (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulfate ester, or reduction or oxidation of a susceptible functionality.

The present invention includes within its scope solvates of the compounds of formula (I) and salts thereof, for example, hydrates.

The present invention also includes within its scope any enantiomers, diastereomers, geometric isomers and tautomers of the compounds of formula (I). It is to be understood that all such isomers and mixtures thereof are encompassed within the scope of the invention.

The present invention further provides a compound of formula (I) or a pharmaceutically acceptable salt thereof for use in therapy.

In another aspect, the invention provides the use of a compound of formula (I) as defined above, or a pharmaceutically acceptable salt thereof, for the manufacture of a medicament for treatment or prevention of infection by hepatitis C virus in a human or animal.

A further aspect of the invention provides a pharmaceutical composition comprising a compound of formula (I) as defined above, or a pharmaceutically acceptable salt thereof, in association with a pharmaceutically acceptable carrier. The composition may be in any suitable form, depending on the intended method of administration. It may for example be in the form of a tablet, capsule or liquid for oral administration, or of a solution or suspension for administration parenterally.

The pharmaceutical compositions optionally also include one or more other agents for the treatment of viral infections such as an antiviral agent, or an immunomodulatory agent such as α-, β- or γ-interferon.

In a further aspect, the invention provides a method of inhibiting hepatitis C virus polymerase and/or of treating or preventing an illness due to hepatitis C virus, the method involving administering to a human or animal (preferably mammalian) subject suffering from the condition a therapeutically or prophylactically effective amount of the pharmaceutical composition described above or of a compound of formula (I) as defined above, or a pharmaceutically acceptable salt thereof. “Effective amount” means an amount sufficient to cause a benefit to the subject or at least to cause a change in the subject's condition.

The dosage rate at which the compound is administered will depend on a variety of factors including the activity of the specific compound employed, the metabolic stability and length of action of that compound, the age of the patient, body weight, general health, sex, diet, mode and time of administration, rate of excretion, drug combination, the severity of the particular condition and the host undergoing therapy. Suitable dosage levels may be of the order of 0.02 to 5 or 10 g per day, with oral dosages two to five times higher. For instance, administration of from 10 to 50 mg of the compound per kg of body weight from one to three times per day may be in order. Appropriate values are selectable by routine testing. The compound may be administered alone or in combination with other treatments, either simultaneously or sequentially. For instance, it may be administered in combination with effective amounts of antiviral agents, immunomodulators, anti-infectives or vaccines known to those of ordinary skill in the art It may be administered by any suitable route, including orally, intravenously, cutaneously and subcutaneously. It may be administered directly to a suitable site or in a manner in which it targets a particular site, such as a certain type of cell. Suitable targeting methods are already known.

An additional aspect of the invention provides a method of preparation of a pharmaceutical composition, involving admixing at least one compound of formula (I) as defined above, or a pharmaceutically acceptable salt thereof, with one or more pharmaceutically acceptable adjuvants, diluents or carriers and/or with one or more other therapeutically or prophylactically active agents.

The present invention also provides a process for the preparation of compounds of formula (I).

According to a general process (A), compounds of formula (I) may be prepared by reacting a compound of formula (II) with a compound of formula (III): 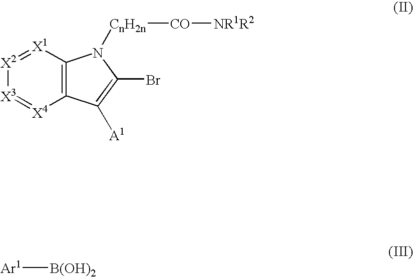
wherein X¹, X², X³, X⁴, R¹, R², A¹ and Ar¹ are as defined for formula (I). The reaction is effected in the presence of a Pd(0) catalyst under conditions typical for the Suzuki reaction.

Alternatively, according to a general process (B), compounds of formula (I) may be prepared by reacting a compound of formula (IV) with a compound of formula (V): 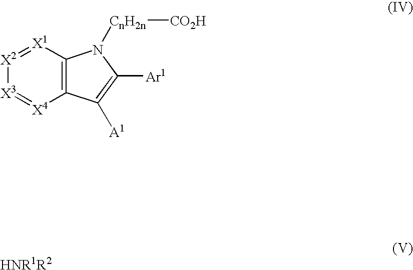
wherein X¹, X², X³, X⁴, A¹, Ar¹, n, R¹ and R² are as defined for formula (I). The reaction is conveniently performed in the presence of a coupling reagent, such as O-(7-azabenzotriazol-1-yl)-N,N,N′,N′-tetramethyluronium hexafluorophosphate, or a coupling reagent on a polystyrene resin, such as PS-carbodiimide, and a base, such as diisopropylethylamine, in a solvent.

Suitable solvents include dimethylformamide and dichloromethane.

Alternatively, according to a general process (C), compounds of formula (I) where X² is CR³ and R³ is C(O)NR⁹R¹⁰ may be prepared by reacting a compound of formula (VI) with a compound of formula (VII): 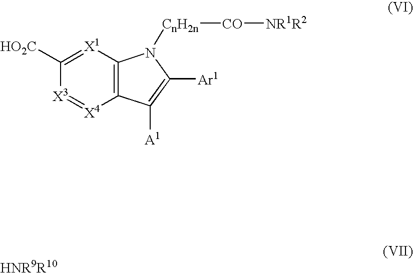
wherein X¹, X³, X⁴, A¹, Ar¹, n, R¹, R², R⁹ and R¹⁰ are as defined for formula (I). The reaction is essentially in the same manner as general process (B).

Alternatively, according to a general process (D), compounds of formula (I), where the (aza)indolyl nitrogen atom is suitably protected, may be prepared by reacting a compound of formula (VIII) with a compound of formula (IX): 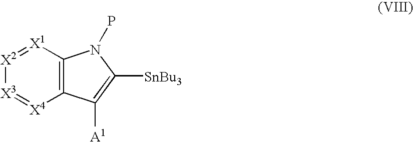
Ar¹—Br (IX)
where P is a suitable protecting group and wherein X¹, X², X³, X⁴, A¹ and Ar¹ are as defined for formula (I). The reaction is effected in the presence of a Pd(0) catalyst, a suitable ligand, such as tri-tert-butylphosphine or triphenylphospine, and a salt, such as caesium fluoride, potassium phosphate or sodium hydrogencarbonate, in a suitable solvent at a temperature between 20° C and the reflux temperature of the solvent. Suitable solvents include organic solvents such as dioxane, dimethoxyethane, tetrahydrofuran or dimethylformamide. Suitable protecting groups include tert-butyloxycarbonyl.

Alternatively, according to a general process (E), compounds of formula (I) where X² is CR³ and R³ is C(O)NR⁹R¹⁰ and R⁹ is SO₂R¹¹ may be prepared by reacting a compound of formula (VI) with a compound of formula (X):
R¹¹O₂S—NHR¹⁰ (X)
wherein X¹, X³, X⁴, A¹, Ar¹, n, R¹, R², R¹⁰ and R¹¹ are as defined for formula (I). The reaction is conveniently effected in the presence of an activator, such as DMAP, and/or a dehydrating agent, such as EDCI in a suitable solvent, such as dichloromethane, dimethylformamide or tetrahydrofuran.

Further details of suitable procedures will be found in the accompanying Examples. For instance, compounds of formula (I) can be converted into other compounds of formula (I) using synthetic methodology well known in the art.

Thus, for instance, the compound of formula (I) where X² is CR³ and R³ is CO₂H may be converted to the compound of formula (I) where R³ is 1H-tetrazol-5-yl by conversion of the carboxylic acid to the amide, for example by treatment with ammonium hydrogen carbonate and di-tert-butyl dicarbonate, followed by conversion of the amide to the nitrile, for example by treatment with triethylamine followed by trifluoroacetic anhydride, and then conversion of the nitrile to the tetrazolyl group using, for example tributyltin azide in a suitable solvent, such as toluene, at a temperature between 20° C. and the reflux temperature of the solvent.

In a similar manner, the compound of formula (I) wherein X² is CR³ and R³ is 5-oxo-4,5-dihydro-1,2,4oxadiazol-3-yl may also be prepared. Thus, following the procedure described above, instead of conversion to the tetrazolyl group, the nitrile group may be converted to the 5-oxo-4,5-dihydro-1,2,4oxadiazolyl group using hydroxylamine followed by carbonyldiimidazole.

Compounds of formulae (II) to (X) are either known compounds or may be prepared by conventional methodology well known to one of ordinary skill in the art using, for instance, procedures described in the accompanying Examples, or by alternative procedures which will be readily apparent.

For example, compounds of formula (II) may be prepared by reacting a compound of formula (XI) with a compound of formula (V): 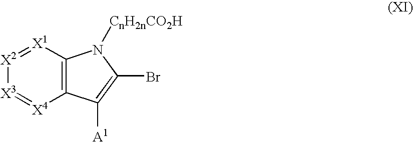
wherein X¹, X², X³, X⁴, A¹ and n are as defined for formula (I). The reaction is essentially effected in the same manner as general process (B).

Compounds of formula (XI) may be prepared by reacting a compound of formula (XII) with a compound of formula (XIII): 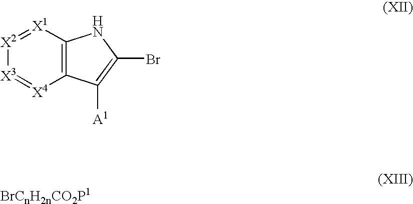
where P¹ is a suitable esterifying group and wherein X¹, X², X³, X⁴, A¹ and n are as defined for formula (I). The reaction is effected by treatment of the compound of formula (XII) with a deprotonating agent, such as sodium hydride, followed by addition of the compound of formula (XIII) and then removal of the ester under suitable conditions. Suitable conditions for deesterification depend on the ester and may include acid or base hydrolysis or hydrogenation. Suitable conditions for acid hydrolysis include trifluoroacetic acid in a suitable solvent, such as dichloromethane.

The compound of formula (XII) where A¹ is cyclohexyl may be prepared from the compound of formula (XIV): 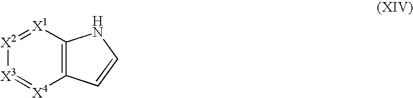
wherein X¹, X², X³ and X⁴ are as defined for formula (I), by alkylation at the 3-position of the compound of formula (XIV) using a deprotonating agent, such as lithium hydride or sodium hydride, followed by 3-bromocyclohex-1-ene, in a suitable solvent, such as DMF. The alkylated product is then hydrogenated to remove the double bond of the cyclohexenyl group. Suitable hydrogenation conditions include the use of hydrogen in the presence of a catalyst, such as palladium on charcoal. The hydrogenated product is then brominated at the 2-position of the (aza)indole ring using a suitable brominating agent, such as NBS.

Compounds of formula (IV) where A¹ is cyclohexyl may be prepared from the compound of formula (XV): 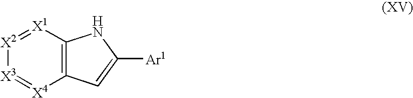
wherein X¹, X², X³, X⁴ and Ar¹ are as defined for formula (I), by alkylation at the 3-position of the compound of formula (XV) using the general process as hereinbefore described for the preparation of the compound of formula (XII). This step is followed by substitution on the nitrogen atom of the (aza)indole system using the general process as hereinbefore described for the preparation of the compound of formula (XI).

Compounds of formula (XV) may be prepared by reacting a compound of formula (XVI) with a compound of formula (XVII): 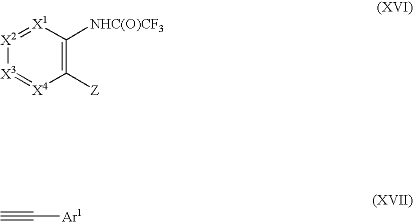
wherein X¹, X², X³, X⁴ and Ar¹ are as defined for formula (I) and Z is Br, I or OTf. The reaction is conveniently effected in a suitable solvent, such as DMF, suitably in the presence of a base, such as tetramethylguanidine, and a catalyst, such as bis-triphenylphosphine palladium(II) chloride/copper(I) iodide.

Compounds of formula (XVI) may be prepared from the compound of formula (XVIII): 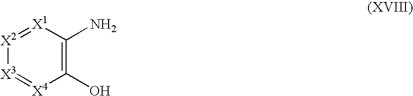
by reaction with trifluoroacetic anhydride followed by treatment with trifluoromethanesulfonyl anhydride.

Compounds of formula (XI) where the carboxylic acid remains protected may alternatively be formed by bromination of the equivalent trimethylsilyl compound. Bromination may be performed using, for example, N-bromosuccinimide, in a suitable solvent. Suitable solvents include halogenated hydrocarbons, such as dichloromethane. The trimethylsilyl equivalent may be formed by the reaction of compound of formula (XIX) with the compound of formula (XX): 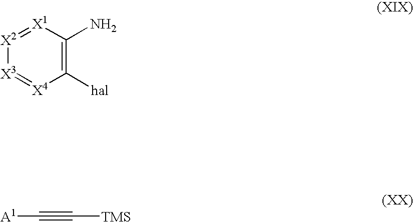
wherein X¹, X², X³, X⁴ and A¹ are as defined for formula (I) and hal represents a suitable halogen atom, such as bromine or iodine.

The reaction is conveniently effected under basic conditions, for example in the presence of sodium carbonate, and in the presence of a Pd(II) salt, such as Pd(dppf)Cl₂, in a suitable solvent, such as DMW. The reaction mixture may be heated, for example in a microwave. The resultant product is then reacted with a compound of formula (XIII) using the process described for the preparation of the compound of formula (XI) to provide the desired product.

Compounds of formula (VIII) can be made by stannylation of the corresponding halo compound, especially the bromo equivalent, using a suitable stannylation agent, such as tri-butyltin chloride, with a lower alkyllithium such as butyllithium.

The compounds of formula (I) where X¹, X³ and X⁴ are CH, X² is CC(O)NHSO₂R¹¹, A¹ is cyclohexyl and n is 1 may be prepared by the transformation shown in Scheme 1:

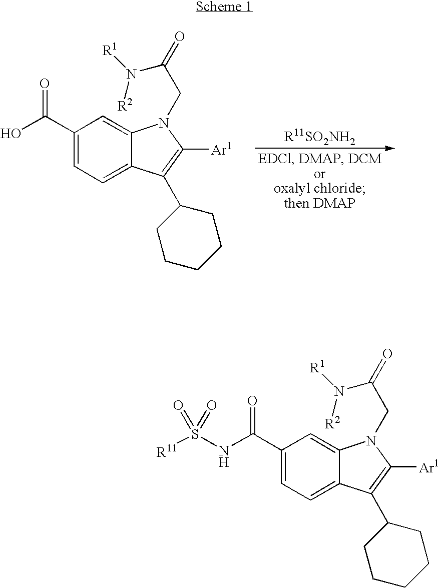

The compounds of formula (I) where X¹, X³ and X⁴ are CH, X² is CR³ and R³ is 5-oxo-4,5-dihydro-1,2,4-oxadiazol-3-yl, A¹ is cyclohexyl and n is 1 may be prepared by the synthetic route shown in Scheme 2:

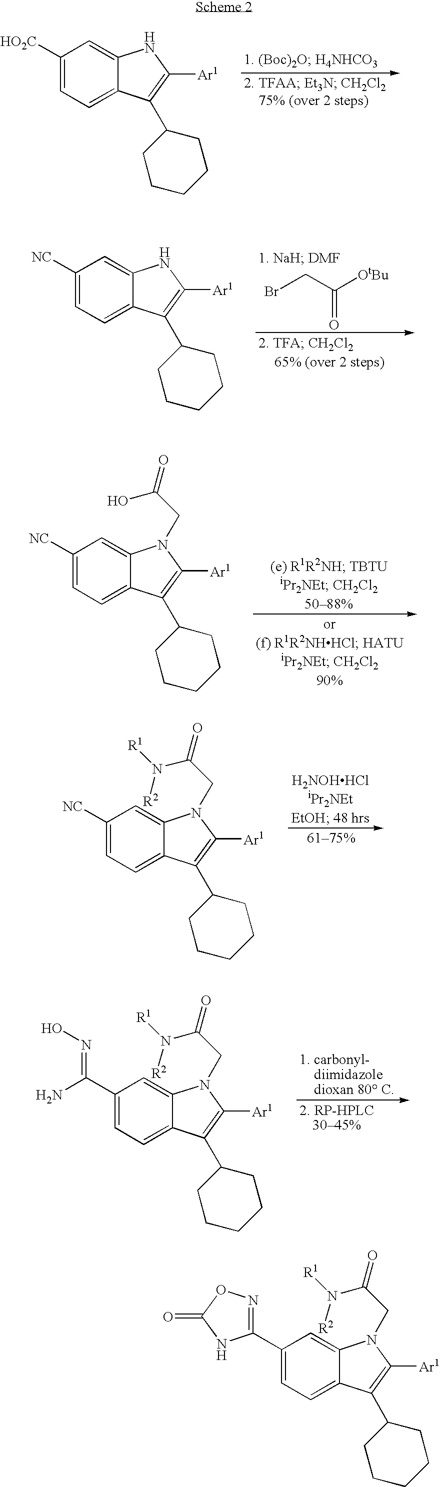

The compounds of formula (Ia) where X² is CCO₂Me may be prepared by the synthetic route shown in Scheme 3:

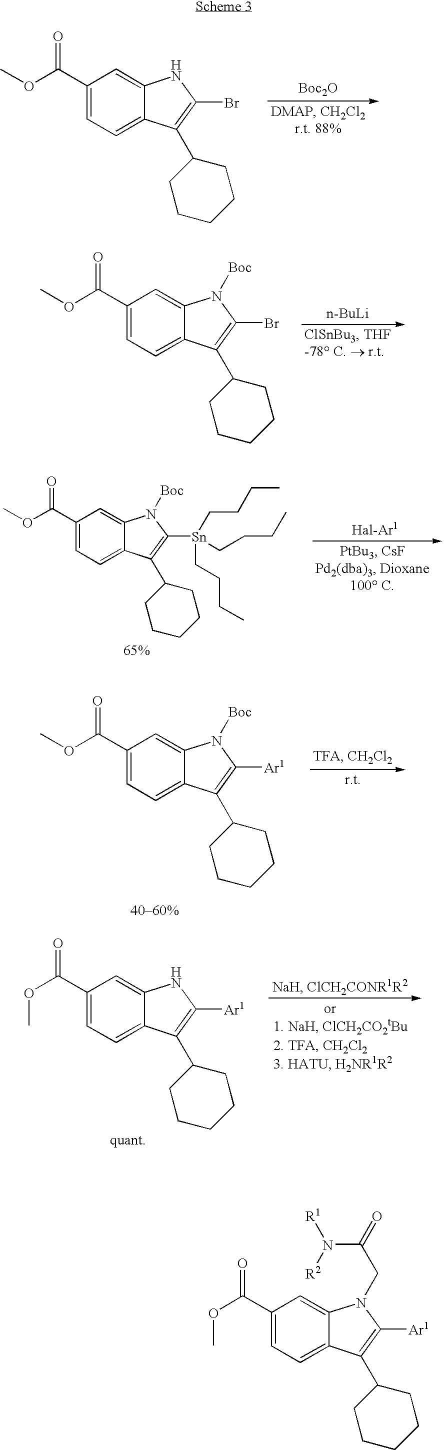

During any of the above synthetic sequences it may be necessary and/or desirable to protect sensitive or reactive groups on any of the molecules concerned.

This may be achieved by means of conventional protecting groups, such as those described in Protective Groups in Organic Chemistry, ed. J. F. W. McOrnie, Plenum Press, 1973; and T. W. Greene & P. G. M. Wuts, Protective Groups in Organic Synthesis, John Wiley & Sons, 3rd edition, 1999. The protecting groups may be removed at a convenient subsequent stage using methods known from the art.

The present invention provides compounds of the formula (I): 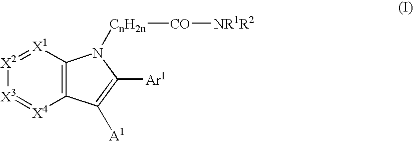
wherein:
Ar¹ is a moiety containing at least one aromatic ring and possesses 5-, 6-, 9- or 10-ring atoms 0 to 3 of which may be N, O or S heteroatoms of which at most 1 will be O or S; which moiety may be optionally substituted by groups Q₁, Q₂ or Q₃ wherein Q₁ is a hydroxy group, or a hydrogen, fluorine, chlorine, bromine or iodine atom or a C₁₋₆ alkyl, C₁₋₆ alkyl substituted by not more than 5 fluorine atoms, C₁₋₆ alkoxyl, C₁₋₆ alkoxyl substituted by not more than 5 fluorine atoms, C₂₋₆ alkenyl or alkynyl, nitro, nitrile, carboxyl, esterified carboxy wherein the esterifying moiety has up to 4 carbon atoms optionally substituted by not more than 5 fluorine atoms,
   Q₂ is a fluorine or chlorine atom or a methyl, trifluoromethyl, methoxy, trifluoromethoxy or difluoromethoxy group.
   Q₃ is a fluorine or chlorine atom or a methyl, methoxyl, trifluoromethoxy or difluoromethoxy group;
   or Ar¹ is a group disclosed as a substituent on the G⁶ moiety of the compound of formula (I) of WO 01/47883 which is incorporated herein by cross reference;
X¹ is N or CR^{a}; X² is N or CR³; X³ is N or CR⁴; X⁴ is N or CR^{b}; with the proviso that at least one of X² and X³ is not N; wherein R^{a} and R^{b} are independently selected from hydrogen, fluorine or chlorine or C₁₋₄alkyl, C₂₋₄alkenyl, C₁₋₄alkoxy, C₁₋₄alkyl or alkoxy optionally substituted by up to 6 fluorine atoms and/or a hydroxyl group;
n is 1, 2, 3, 4, 5 or 6;
R¹ and R² are independently hydrogen, a group Ar², C₁₋₆ alkyl, C₂₋₆ alkenyl or a C₁₋₆ alkyl or C₂₋₆ alkenyl group substituted by 1-3 fluorine atoms or a OR⁷, NR⁷R⁸, CO₂H, Ar² or A² group or R¹ and R² are joined to form a ring of 3 to 8 ring atoms, 1 or 2 of which ring atoms may be selected from N, O, S, SO, or SO₂ moieties, which ring may be substituted by a group Ar², A², C₁₋₆ alkyl, C₁₋₆ alkyl Ar²,C₁₋₆alkyl A², or a further ring of 5-6 ring atoms 1 or 2 of which may be selected from N, O, S which further ring may be substituted by C₁₋₆ alkyl substituted by 1-3 fluorine atoms, OR⁷, NR⁷R⁸ or CO₂H group; R⁷ is hydrogen or C₁₋₆ alkyl, R⁸ is hydrogen, C₁₋₄ alkyl optionally substituted by hydroxy, carboxy, amino, monoC₁₋₆ alkyl or diC₁₋₆ alkyl wherein the alkyl groups may be joined to form a 5- or 6-membered unsaturated ring which may contain a O, S, NH or NCH₃ group;
Ar² is a moiety containing at least one aromatic ring and possesses 5-, 6, 9- or 10-ring atoms 0 to 3 of which atoms may be N, O or S heteroatoms of which at most 1 will be O or S; which aromatic ring may be optionally substituted by groups Q₁′, Q₂′ or Q₃′ wherein Q₁′ is a hydroxy group, or a hydrogen, fluorine, chlorine, bromine or iodine atom or a C₁₋₆ alkyl, C₁₋₄ alkyl substituted by not more than 5 fluorine atoms, C₁₋₆ alkoxyl, C₁₋₄ alkoxyl substituted by not more than 5 fluorine atoms, C₂₋₆ alkenyl or alkynyl, nitro, nitrile, carboxyl, esterified carboxy wherein the esterifying moiety has up to 4 carbon atoms optionally substituted by not more than 5 fluorine atoms,
   Q₂′ is a fluorine or chlorine atom or a methyl, trifluoromethyl, methoxy, trifluoromethoxy or difluoromethoxy group.
   Q₃′ is a fluorine or chlorine atom or a methyl, methoxyl; trifluoromethoxy or difluoromethoxy group;
A¹ is C₁₋₆ alkyl, C₂₋₆ alkenyl, or C₁₋₆ alkyl or C_{2- 6} alkenyl substituted by C₁₋₄ alkoxy or up to 5 fluorine atoms or a non-aromatic ring of 3 to 8 ring atoms which may contain a double bond and which may contain a O, S, SO, SO₂ or NH moiety and which may be optionally substituted by one or two alkyl groups of up to 2 carbon atoms or by 1 to 8 fluorine atoms;
A² is C₁₋₆ alkyl, C₂₋₆ alkenyl, or C₁₋₆ alkyl or C₂₋₆ alkenyl substituted by C₁₋₄ alkoxy or up to 5 fluorine atoms or a non-aromatic ring of up to 8 ring atoms which may contain a double bond and which may contain a O, S, SO, SO₂ or NH moiety and which may be optionally substituted by one or two alkyl groups of up to 2 carbon atoms or by 1 to three fluorine atoms;
one of R³ and R⁴ is a Het or is hydrogen, fluorine, chlorine or bromine atom or a C₁₋₄ alky, C₂₋₄ alkenyl, C₁₋₄ alkoxy, C₁₋₄ alkyl or alkoxy substituted by up to 5 fluorine atoms, nitrile, carboxy, C₁₋₄ alkoxycarbonyl, C₁₋₄ alkyl or C₂₋₄ alkenyl substituted by a carboxy or C₁₋₄ alkoxycarbonyl group, or a SO₂NR⁹R¹⁰ or CONR⁹R¹⁰ group where R⁹ is hydrogen, C₁₋₄ alkyl, SO₂R¹¹ or COR¹¹ and R¹⁰ is hydrogen, hydroxyl or C₁₋₄ alkyl or R⁹ and R¹⁰ are alkylene linked to form a 5- or 6-membered ring, and R¹¹ is C₁₋₄ alkyl optionally substituted by up to 5 fluorine atoms or a group independently chosen from within the definitions of the Ar² group;
   Het is a 5 or 6-membered aromatic ring 1, 2 or 3 of which may be selected from N, O, S which ring may be substituted by 1 or 2 groups selected C₁₋₄ alkyl or hydroxy or tautomers thereof, or is 2-hydroxy-cyclobutene-3,4-dione;
the other of R³ and R⁴ is a hydrogen, fluorine or chlorine atom or C₁₋₄ alkyl, C₂₋₄ alkenyl, C₁₋₄ alkoxy, C₁₋₄ alkyl or alkoxy substituted by up to 6 fluorine atoms and optionally a hydroxyl; and
or a pharmaceutically acceptable salt thereof.

The group CₙH₂ₙ may be straight or branched such as a —CH₂—, —(CH₂)₂—, —(CH₂)₃—, —(CH₂)₄—, —CH(CH₃)—, —CH₂—CH(CH₃)—, —CH(CH₃)—CH₂— or the like straight or branched butyl, pentyl or hexyl group. Most suitably the CₙH₂ₙ group is a —CH₂— group.

When used herein C₁₋₆ alkyl means methyl, ethyl, 1-propyl, 2-propyl or a straight or branched butyl, pentyl or hexyl group. Particularly apt C₁₋₆ alkyl groups are methyl, ethyl, propyl and butyl groups. Favoured alkyl groups are ethyl and methyl groups. The methyl group is the preferred alkyl group.

Most suitably a C₁₋₆ alkyl group substituted by up to 5 fluorine atoms will include a CF₃, CHF₂ and/or CF₂ moiety. Favoured fluoroalkyl groups are the CF₃, CH₂F and CF₂CF₃ groups. The CF₃ group is the preferred fluoroalkyl group.

When used herein C₂₋₆ alkenyl means a —CH═CH₂, —C(CH₃)═CH₂, —CH═C(CH₃), —C(CH₃)═C(CH₃) or straight or branched pentylene or hexylene groups.

When used herein C₁₋₆ alkoxy and fluorinated C₁₋₆ alkoxy are analogous to the alkyl and fluoroalkyl groups described above so that, for example, preferred groups include OCH₃, OCF₃ and OCHF₂ groups.

Favoured values for R^{a} and R^{b} independently include hydrogen, fluorine, methyl, methoxy and trifluoromethyl. Particularly apt values for R^{a} and R^{b} include hydrogen or fluorine. A preferred value for R^{a} is hydrogen. A preferred value for R^{b} is hydrogen.

The Ar¹ moiety may contain a single aromatic ring or one aromatic ring to which a further aromatic or non-aromatic ring is fused.

Ar¹ is aptly phenyl, naphthyl, indinyl, tetrahydronaphthyl, pyridyl, furyl, thienyl, pyrolidyl, oxazolyl, thiazolyl, pyrazolyl, pyridazolyl, triazolyl, oxadiazolyl, thiodiazolyl or quinonyl, any of which may be optionally substituted by group Q¹, Q² or Q³ as hereinbefore defined.

Favourably, Ar¹ is a furyl or thienyl group or a group of the formula C₆H₂Q¹Q²Q³. One particularly favoured group Ar¹ is the furyl group, Other particularly favoured Ar¹ groups are optionally substituted phenyl groups of the formula C₆H₃Q¹Q² of which phenyl, fluorophenyl, chlorophenyl, hydroxyphenyl, trifluoromethylphenyl, methoxyphenyl, difluorophenyl and the like are preferred.

Particularly suitable groups A¹ include those groups of the formula 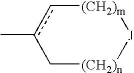
wherein m+n is 0, 1, 2, 3 or 4, preferably 1 or 2, the dotted line represents an optional double bond and J is CH₂, O, S, SO, SO₂ or NH which group of the above formula may optionally be substituted by one or two methyl groups.

Favoured groups A¹ include cycloalkyl and cycloalkenyl groups of 5 or 6 ring members.

A preferred group A¹ is the cyclohexyl group.

Particularly apt compounds of this invention include those wherein one of R³ and R⁴ is a carboxy or —Y—CO₂H group wherein Y is CH₂, CH₂CH₂ or CH:CH group, or a pharmaceutically acceptable salt thereof.

A preferred group R³ is the CO₂H group or a pharmaceutically acceptable salt thereof.

Favourably, one of R³ and R⁴ is a hydrogen atom.

Certain favoured compounds of the invention include those wherein R⁴ is hydrogen, fluorine or chlorine of which hydrogen is preferred.

A favoured value for X₄ is CH.

In those compounds of formula (I) wherein R¹ is a hydrogen atom or C₁₋₄alkyl group, R² may aptly be a hydrogen atom or a C₁₋₄ alkyl or a group C₁₋₄ alkyl Ar² group wherein the Ar² group is as hereinbefore defined wherein Q² and Q³ are hydrogen atoms.

In those compounds of formula (I) wherein R¹ and R² are linked, they aptly form an optionally substituted ring of the formula: 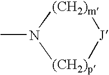
wherein J′ is CH₂, NH, O, S, SO, or SO₂ and m′+p′ is 1 to 6, more aptly 2 to 5 and preferably 3 or 4 and where the one or two optional substituents are selected from C₁₋₄ alkyl and hydroxy and Ar² where the Ar² group is as hereinbefore defined or a fused pendent or spiro 5 or 6 membered ring in which one of the ring moieties may be a O, NH or NCH₃ group.

Favoured values for A¹ include non-aromatic rings. Such rings are aptly of 5 or 6 carbon atoms and which are saturated or monounsaturated. Preferred groups A¹ include cyclopentyl, cyclohexyl and cyclohexenyl groups.

Certain particularly suitable compounds of the invention are represented by the formula (II): 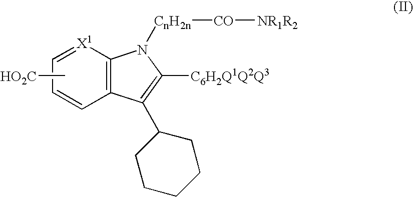
wherein n, X¹, Q¹, Q², Q³, R¹ and R² are as defined in relation to formula (I) or a pharmaceutically acceptable salt thereof.

In compounds of formulae (I) and (II), a favoured value for Q³ is H, a favoured value for n is 1 and a favoured value for X¹ is CH so that particularly apt compounds of the invention include those of formula (III): 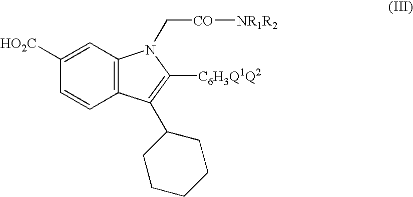
wherein Q¹, Q², R¹ and R² are defined in relation to formula (I), or a pharmaceutically acceptable salt thereof.

In certain apt compounds of formulae (II) and (III), Q² is hydrogen fluorine chlorine, methyl, methoxyl or trifluoromethyl. In certain apt compounds of formulae (II) and (III) Q¹ is hydrogen or fluorine. In certain preferred compounds of formulae (II) and (III) Q¹ is hydrogen and Q² is hydrogen or fluorine.

In compounds of formulae (I), (II) and (III), particularly apt values for NR¹R² are those wherein R¹ is hydrogen or methyl, R² is hydrogen, methyl or ethyl optionally substituted by (i) an aryl group of 5 or 6 ring atoms up to 3 of which may be selected from O, N or S of which not more than one may be O or S which aryl group may be substituted by a methyl or methoxy group; (ii) a 5 or 6 membered saturated ring which one ring atom may be a O, S or N atom and which ring may be substituted by a methyl group; or (iii) 2-substituted by a hydroxy, amino, methylamino or dimethylamino group; or R¹ and R² may be joined so that NR¹R² forms a 4 or 6 membered saturated ring of which one additional ring atom may be a O, S or N atom and which ring may be substituted by a methyl group.

In compounds of formulae (I), (II) and (III), particularly suitable —NR¹R³ groups include (wherein Py is pyridyl):
—NH₂, —NH—CH₃, —NH—C₂H₅, —N(CH₃)₂, —N(CH₃)C₂H₅, —NHCH₂C₆H₅, —NH—CH₂C₆H₄F, —NH—CH₂C₆H₄OCH₃, —N(1CH₃)CH₂C₆H₅, —N(CH₃)CH₂C₆H₄F, —N(CH₃)CH₂C₆H₄OCH₃, 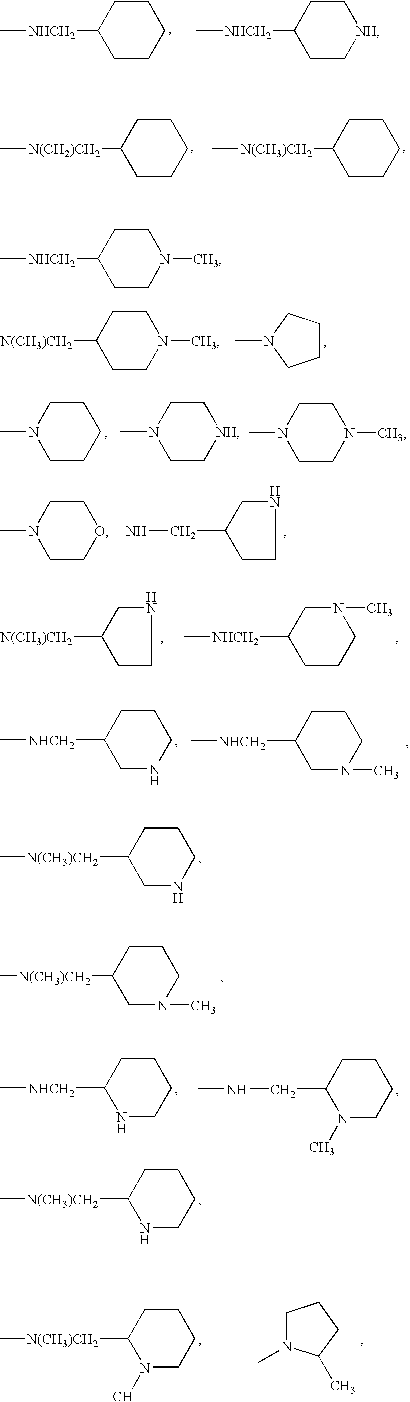 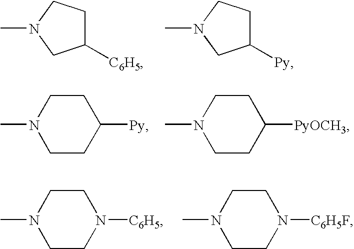
—NH—C(CH₅)C₆H₅,—NH—C(CH₃)Py, —N(CH₃)C(CH₃)C₆H₅, —NH—CH₂CH:CH₂, —NH— CH₂C≡CH, N(CH₃)CH₂CH:CH₂, —N(CH₃)CH₂C≡CH, 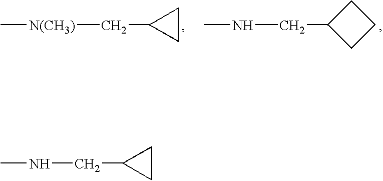
—NH—CH₂CH₂NH₂, —NH—CH₂CH₂—N(CH₃)₂, —NH—CH₂CH₂—NHCH₃, N(CH₃)—CH₂CH₂NH₂, —N(CH₃)CH₂CH₂N(CH₃)₂, —NH—CH₂CH₂OH.

The compounds of the formula (I) may be in the form of a pharmaceutically acceptable salt such as a sodium, potassium, calcium, magnesium or ammonium salt or a salt with a pharmaceutically acceptable organic base. If the compounds of the formula (I) also contain a group, the compound may be zwitterionic or in the form of a salt with a pharmaceutically acceptable acid such as hydrochloric, sulfuric, phosphoric, methanesulfonic and the like acid.

The present invention provides a process for the preparation of compounds of formula (I) and their salts which comprises the reaction of compounds of the formulae (IV) and (V): 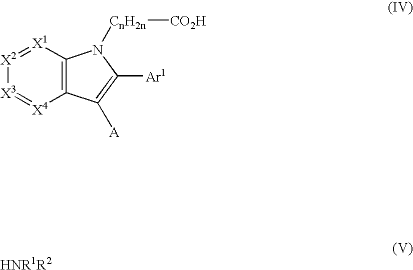
In the compounds of formulae (IV) and (V) any reactions group that requires masking during the amidation reaction may be protected in conventional manner and the protecting group removed thereafter.

This principle of utilising protecting groups also applies to all other reactions described hereinafter. For example, if the desired compound of the formula I contains a CO₂H group, then the compound of the formula (lV) may contain a CO₂CH₃ group and the resulting compound of the formula (I) may be hydrolysed in conventional manner, for example with sodium hydroxide in aqueous methanol or BBr₃ in DCM to yield the compound containing the carboxylate or its sodium salt. Similarly the substituents on the core bicycle may be elaborated after the amidation reaction, for example if the desired compound of formula (I) contains a tetrazole group then the compound of formula (IV) may contain CN group and the resulting compound of formula (I) may be reacted with an azide.

The compound of the formula (IV) may be prepared from the corresponding compound of the formula (VI): 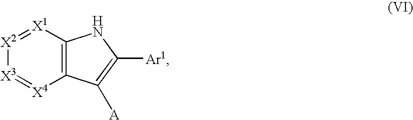
by reaction with 1-bromo ethanoic acid t-butyl ester under conventional conditions for forming an amide followed by de-esterification with trifluoroethanoic acid in DCM.

In an alternative process the compounds of formula (I) may be prepared from the corresponding compound of the formula (VII): 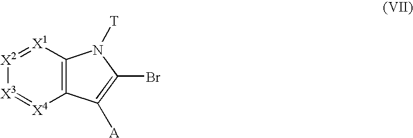
wherein T is a CₙH₂ₙCONR¹R² group by reaction with Ar¹B(OH)₂ in the presence of a Pd[O] catalyst under conditions conventional for the Suzuki reaction.

The compound of formula (VII) wherein T is a CₙH₂ₙCONR¹R² group can be prepared from the compound of formula (VII) wherein T is a hydrogen atom by reaction with 1-bromoethanoic acid t-butyl ester.

Alternatively, the compound of formula (VII) may be prepared by the reaction of NBS and the compound of the formula (VIII): 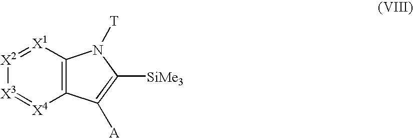
wherein T is CₙH₂ₙCONR¹R² which may itself be prepared from the corresponding compound of formula (VIII) wherein T is H by reaction with BrCₙH₂ₙCONR¹R² under conventional alkylation conditions.

In an alternative synthesis the compounds of the formula (VI) may be prepared from the reaction of corresponding compounds of the formulae (IX) and (X):

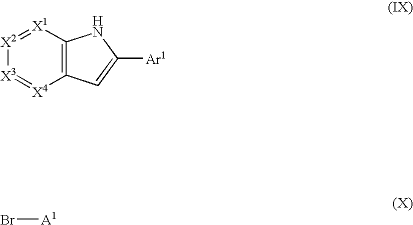

Similarly, certain compounds of the formula (XI) may be prepared by the reaction of a compound of the formula (IX) with compounds of the formula (XII): 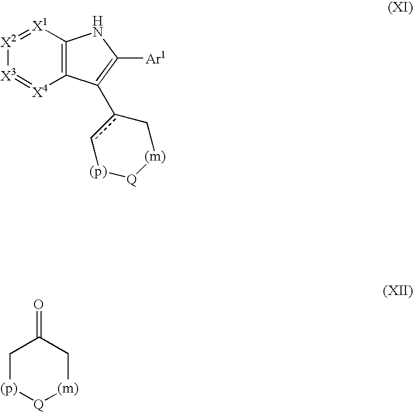
wherein Q is CH₂, NH, O, S, SO or SO₂ and m+p is 1 or 2 and where one or two optional substituents are selected from C₁₋₆ alkyl and hydroxyl and the dotted line is an optional double bond; optionally followed by reduction of said optional double bond.

The compounds of formula (XI) may also be prepared by the reaction of the compounds of the formulae (XIII) and (XIV): 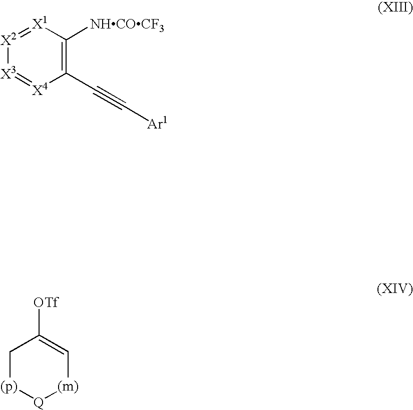
wherein Q, m and p are as defined in relation to formula (XII) in the presence of a Pd[O] catalyst optionally followed by reduction of the optional double bond.

The compound of the formula (XIII) may be prepared from the compounds of the formulae (XV) and (XVI): 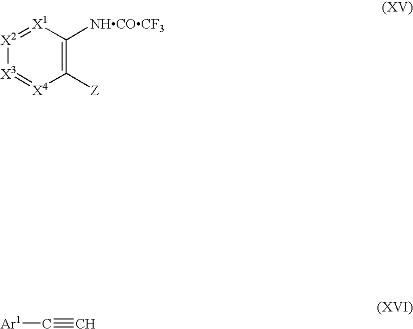
wherein Z is I, Br or OTf in the presence of a Pd[O] catalyst.

A further process for the preparation of the compounds of formula (VIII) wherein T is hydrogen comprises the reaction of the compounds of the formulae: 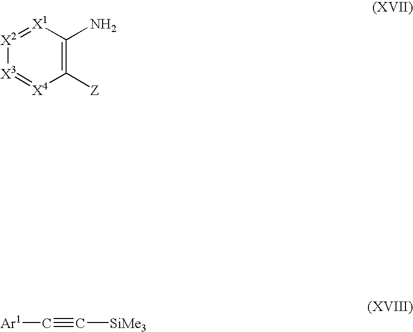
wherein Z is I, Br or OTf.

In addition, compounds of the formula (VI) may be prepared by the reaction of a hydrazine of the formula (XIX): 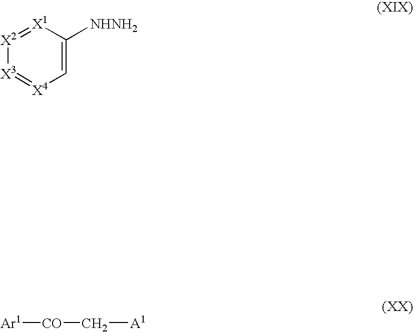
and a ketone of the formula (XX).

The compounds of formulae (I)-(III) may be used for the inhibition of HCV polymerase and so may be used for the manufacture of medicaments which may be used to treat HCV infection.

Accordingly, this invention provides a pharmaceutical composition comprising a compound of the formula (I) as hereinbefore described as a pharmaceutically acceptable salt thereof together with a pharmaceutically acceptable carrier.

The invention also provides pharmaceutical compositions comprising one or more compounds of this invention in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. Typical unit dosage forms contain from 1 to 100 mg, for example 1, 2, 5, 10, 25, 50 or 100 mg, of the active ingredient. The tablets or pills of the novel composition can be coated or otherwise compounded to provide a dosage form affording the advantage of prolonged action.

The liquid forms in which the novel compositions of the present invention may be incorporated for administration orally or by injection include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

In the treatment of infection due to hepatitis C, a suitable dosage level is about 0.01 to 250 mg/kg per day, preferably about 0.05 to 100 mg/kg per day, and especially about 0.05 to 5 mg/kg per day. The compounds may be administered on a regimen of 1 to 4 times per day. Most suitably the administration is orally using a unit done as previously indicated.

In a further aspect this invention provides the use of a compound of formula (I) or a pharmaceutically acceptable salt thereof in the manufacture of a medicament for the treatment of infection by hepatitis C virus. Most suitably the medicament is in unit dose form adapted for oral administration as indicated hereinbefore.

In another aspect this invention provides the use of a compound of formula (I) or a pharmaceutically acceptable salt thereof for the treatment of infection by hepatitis C virus in a mammal and preferably in a human. Most suitably the treatment is effected by oral administration of a unit dose form as indicated hereinbefore.

Useful references in the literature for synthetic preparations include: Nanomoto et al, J. Chem. Soc. Perkin I. 1990, III; Freter, S. Org. Chem., 1975, 40, 2525; Cacchi et al, Eur. J. Org. Chem., 2002, 2671; Ujjainwalla, Tetrahedron Lett., 1998, 39, 5355; Wang et al, J. Org. Chem., 2000, 65, 1889; Larock, J. Org. Chem., 1998, 63, 7652; Kelly et al, J. Org. Chem., 1996, 61, 4623; and Cacchi, Tetrahedron Lett., 1992, 33, 3915.

The following Examples illustrate the preparation of compounds according to the invention.

The compounds of the invention were tested for inhibitory activity against the HCV RNA dependent RNA polymerase (NS5B) in an enzyme inhibition assay (example i)) and a cell based sub-genomic replication assay (describe in example ii)). The compounds generally have IC50's below 5 μM in the enzyme assay and EC50's below 20 μM in the cell based assay. For example, 3-cyclohexyl-1-(2-(methyl[(1-methylpiperidin-2-yl)methyl]amino}-2-oxoethyl)-2-phenyl-1H-indole-6-carboxylic acid trifluoroacetate had an IC₅₀ of 14 nM in the enzyme assay and an EC50 of 270 nM in the cell based assay.

i) In-vitro HCV NS5B Enzyme Inhibition Assay

WO 96/37619 describes the production of recombinant HCV RdRp from insect cells infected with recombinant baculovirus encoding the enzyme. The purified enzyme was shown to possess in vitro RNA polymerase activity using RNA as template. The reference describes a polymerisation assay using poly(A) and oligo(U) as a primer or an heteropolymeric template. Incorporation of tritiated UTP or NTPs is quantified by measuring acid-insoluble radioactivity. The present inventors have employed this assay to screen the various compounds described above as inhibitors of HCV RdRp.

Incorporation of radioactive UMP was measured as follows. The standard reaction (50 μl) was carried out in a buffer containing 20 mM tris/HCl pH 7.5, 5 mM MgCl₂, 1 mM DTT, 50 mM NaCl, 0.03% N-octylglucoside, 1 μCi [³H]-UTP (40 Ci/mmol, NEN), 10 μM UTP and 10 μg/ml poly(A) or 5 μM NTPs and 5 μg/ml heteropolymeric template. Oligo(U)₁₂ (1 μg/ml, Genset) was added as a primer in the assay working on Poly(A) template. The final NS5B enzyme concentration was 5 nM. The order of assembly was: 1) compound, 2) enzyme, 3) template/primer, 4) NTP. After 1 h incubation at 22° C. the reaction was stopped by adding 50 μl of 20% TCA and applying samples to DE81 filters. The filters were washed thoroughly with 5% TCA containing 1M Na₂HPO_{4/}NaH₂PO₄, pH 7.0, rinsed with water and then ethanol, air dried, and the filter-bound radioactivity was measured in the scintillation counter. Carrying out this reaction in the presence of various concentrations of each compound set out above allowed determination of IC₅₀ values by utilising the formula:
% Residual activity=100/(1+[I]/IC₅₀)^{s}
where [I] is the inhibitor concentration and “s” is the slope of the inhibition curve.
ii) Cell based HCV Replication Assay

Cell clones that stably maintain subgenomic HCV replicon were obtained by transfecting Huh-7 cells with an RNA replicon identical to I₃₇₇neo/NS3-3′/wt described by Lohmann et al. (1999) (EMBL-genbank No. AJ242652), followed by selection with neomycin sulfate (G418). Viral replication was monitored by measuring the expression of the NS3 protein by an ELISA assay performed directly on cells grown in 96 wells microtiter plates (Cell-ELSA) using the anti-NS3 monoclonal antibody 10E5/24 (as described by De Francesco, Raffaele; Migliaccio, Giovanni; Paonessa, Giacomo. Hepatitis C virus replicons and replicon enhanced cells. PCT Int. Appl. WO 0259321 A2 20020801). Cells were seeded into 96 well plates at a density of 10⁴ cells per well in a final volume of 0.1 ml of DMEM 10% FCS. Two hours after plating, 50 μl of DMEM/10% FCS containing a 3× concentration of inhibitor were added, cells were incubated for 96 hours and then fixed for 10′ with ice-cold isopropanol. Each condition was tested in duplicate and average absorbance values were used for calculations. The cells were washed twice with PBS, blocked with 5% non-fat dry milk in PBS+0.1% Triton X100+0.02% SDS (PBSTS) and then incubated o/n at 40° C. with the 10E5/24 mab diluted in Milk/PBSTS. After washing 5 times with PBSTS, the cells were incubated for 3 hours at room temperature with Fc specific anti-mouse IgG conjugated to alkaline phosphatase (Sigma), diluted in Milk/PBSTS. After washing again as above, the reaction was developed with p-Nitrophenyl phosphate disodium substrate (Sigma) and the absorbance at 405/620 nm read at intervals. For calculations, we used data sets where samples incubated without inhibitors had absorbance values comprised between 1 and 1.5. The inhibitor concentration that reduced by 50% the expression of NS3 (IC₅₀) was calculated by fitting the data to the Hill equation,
Fraction inhibition=1−(Ai-b)/(A₀-b)=[I]ⁿ/([I]ⁿ+IC₅₀)
where:
Ai=absorbance value of HBI10 cells supplemented with the indicated inhibitor concentration.
A₀=absorbance value of HBI10 cells incubated without inhibitor.
b=absorbance value of Huh-7 cells plated at the same density in the same microtiter plates and incubated without inhibitor.
n=Hill coefficient.
   iii) General Procedures

All solvents were obtained from commercial sources (Fluka, puriss.) and were used without further purification. With the exception of routine deprotection and coupling steps, reactions were carried out under an atmosphere of nitrogen in oven dried (110° C.) glassware. Organic extracts were dried over sodium sulfate, and were concentrated (after filtration of the drying agent) on rotary evaporators operating under reduced pressure. Flash chromatography was carried out on silica gel following published procedure (W. C. Still et al., J. Org. Chem. 1978, 43, 2923) or on automated or semi-automated flash chromatography systems utilising pre-packed columns.

Reagents were usually obtained directly from commercial suppliers (and used as supplied) but a limited number of compounds from in-house corporate collections were utilised In the latter case the reagents are readily accessible using routine synthetic steps that are either reported in the scientific literature or are known to those skilled in the art.

¹H nmr spectra were recorded on Bruker AM series spectrometers operating at (reported) frequencies between 300 and 600 MHz. Chemical shifts (δ) for signals corresponding to non-exchangeable protons (and exchangeable protons where visible) are recorded in parts per million (ppm) relative to tetramethylsilane and are measured using the residual solvent peak as reference. Signals are tabulated in the order: multiplicity (s, singlet; d, doublet; t, triplet, q, quartet; ma, multiplet; br, broad, and combinations thereof); coupling constant(s) in hertz; number of protons. Mass spectral (MS) data were obtained on a Perkin Elmer API 100 operating in negative (ES⁻) or positive (ES⁺) ionization mode and results are reported as the ratio of mass over charge (m/z) for the parent ion only. Preparative scale HPLC separations were carried out on a Waters Delta Prep 4000 separation module, equipped with a Waters 486 absorption detector or on a Thermoquest P4000 equipped with a UV1000 absorption detector. In all cases compounds were eluted with linear gradients of water and acetonitrile both containing 0.1% TFA using flow rates between 15 and 25 mL/min.

The following abbreviations are used in the examples, the schemes and the tables: DIEA: diisopropylethyl amine; DUT: dimethylformamide; DMSO: dimethylsulfoxide; eq.: equivalent(s); AcOEt: ethyl acetate; Et₂O: diethyl ether; MeCN: acetonitrile; h: hour(s); HATU: O-(7-azabenzotriazol-1-yl)-N,N,N′,N′-tetramethyluronium hexafluorophosphate; Me: methyl; EtOH: ethanol; min: minutes; NBS: N-bromo succinimide; Ph: phenyl; HPLC: reversed phase high-pressure liquid chromatography; TEA: trifluoroacetic acid; TBF: tetrahydrofuran; MeOH: methanol; DME: Ethylene glycol dimethyl ether; TMS: trimethylsilyl; EDCI: 1-(3-dimethylaminopropyl)-3-ethylcarbodiimide hydrochloride.

### EXAMPLE 1

### 3-cyclohexyl-1-[2-(diethylamino)-2-oxoethyl]-2-(4-methylphenyl)-1H-indole-6-carboxylic acid

Step 1: methyl 3-cyclohex-2-en-1-yl-1H-indole-6-carboxylate

A solution (0.2 M) of methyl 1H-indole-6-carboxylate in DMF was cooled to 0° C. then treated with LiH (1.3 eq.). The mixture was stirred for 0.5 h then warmed to 20° C. A solution (1.0 M) of 3-bromocyclohex-1-ene (1.5 eq.) in DMF was added and the mixture was stirred for 16 h. AcOEt and H₂O were added and the organic layer was separated then washed with aqueous HCl (1 N) and dried. Removal of the solvent gave a residue that was purified by flash chromatography on silica gel (5:95 AcOEt/petroleum ether) to afford the title compound (52%) as an oil.

¹H NMR (300 MHz, CDCl₃, 300 K) δ 1.61-1.85 (m, 4H), 2.05-1.18 (m, 2H), 3.71-3.75 (m, 1H), 3.94 (s, 3H), 5.80-5.95 (m, 2H), 7.14 (s, 1H), 7.67 (d, J 8.4 H), 7.80 (d, J 8.4 Hz, 1H), 8.12 (s, 1H), 8.20 (br s, 1H); MS (ES⁺) m/z 256 (M+H)⁺.

Step 2: methyl 3-cyclohexyl-1H-indole-6-carboxylate

A solution (0.2 M) of methyl 3-cyclohex-2-en-1-yl-1H-indole-6-carboxylate (from Step 1) in MeOH was treated with 10% Pd/C (10% wt.). The resulting suspension was stirred for 4 h under an atmosphere of hydrogen then purged with nitrogen and filtered. The filtrate was concentrated to afford the title compound (97%) as a solid.

¹H NMR (400 MHz, DMSO-d₆, 300 K) δ 1.22-1.24 (m, 2H), 1.39-1.51 (m, 3H), 1.69-1.81 (m, 3H), 1.95-2.00 (m, 2H), 2.75-2.81 (m, 1H), 3.83 (s, 3H), 7.33 (s, 1H), 7.57 (d, J 8.4 Hz, 1H), 7.63 (d, J 8.4 Hz, 1H), 8.00 (s, 1H), 11.16 (br s, 1); MS (ES⁺) m/z 258 (M+H)⁺.

Step 3: methyl 2-bromo-3-cyclohexyl-1H-indole-6-carboxglate

A solution (0.1 M) of methyl 3-cyclohexyl-1H-indole-6-carboxylate (from Step 2) in CCl₄, was treated with NBS (1.1 eq.). The resulting mixture was stirred at 40° C. for 2 h, then the reaction was quenched by addition of 10% aqueous Na₂S₂O₄. The organic phase was separated and washed with brine, then dried. Removal of the solvent afforded a residue that was purified by flash chromatography on silica gel (5:95 AcOEt/petroleum ether) to afford the title compound (54%) as a solid.

¹H NMR (300 MHz, DMSO-d₆, 300 K) δ 1.32-1.49 (m, 3H), 1.64-2.00 (m, 7H), 2.73-2.88 (m, 1H), 3.84 (s, 3H), 7.61 (d, J 8.4 Hz, 1H), 7.78 (d, J 8.4 Hz, 1H), 7.90 (s, 1H), 12.02 (br s, 1H); MS (ES⁺) m/z 336 (M+H)⁺.

Step 4: methyl 2-bromo-1-(2-tert-butoxy-2-oxoethyl)-3-cyclohexyl-1H-indole-6-carboxylate

A solution (0.1 M) of methyl 2-bromo-3-cyclohexyl-1H-indole-6-carboxylate (from Step 3) in DMF was treated with NaH (1.3 eq.) and stirred for 0.5 h at 0° C. The solution was warmed to room temperature and treated with tert-butylbromoacetate (1.2 eq.) over 0.5 h. The mixture was stirred for 12 h then diluted with AcOEt and washed sequentially with aqueous HCl (1 N) and brine. The dried organic phase was concentrated and the residue purified by flash chromatography on silica gel (5:95 AcOEt/petroleum ether) to afford the title compound (83%) as a solid.

¹H NMR (300 MHz, DMSO-d₆, 300 K) δ 1.31-1.50 (m, 3H), 1.40 (s, 9H), 1.64-1.80 (m, 3H), 1.81-2.04 (m, 4H), 2.80-2.92 (m, 1H), 3.86 (s, 3H), 5.09 (s, 2H), 7.66 (d, J 8.4 Hz, 1H), 7.82 (d, J 8.4 Hz, 1H), 8.13 (s, 1H); MS (ES⁺) m/z 452 (M+H)⁺.

Step 5: [2-bromo-3-cyclohexyl-6-(methoxycarbonyl)-1H-indol-1-yl]acetic acid

A solution (0.05 M) of methyl 2-bromo-1-(2-tert-butoxy-2-oxoethyl)-3-cyclohexyl-1H-indole-6-carboxylate (from Step 4) in a 1:1 mixture of CH₂Cl₂/TFA was stirred for 16 h. The mixture was concentrated and the residue triturated with Et₂O to afford the title compound (95%) as a solid.

¹H NMR (300 MHz, DMSO-d₆, 300 K) δ 1.30-1.50 (m, 3H), 1.64-1.78 (m, 3H), 1.79-2.02 (m, 4H), 2.79-2.90 (m, 1H), 3.86 (s, 3H), 5.10 (s, 2H,), 7.67 (d, J 8.4 Hz, 1H), 7.83 (d, J 8.4 Hz, 1H), 8.13 (s, 1H).

Step 6: methyl 2-bromo-3-cyclohexyl-1-[2-(dimethylamino)-2-oxoethyl]-1H-indole-6-carboxylate

A solution (0.2 M) of [2-bromo-3-cyclohexyl-6-(methoxycarbonyl)-1H-indol-1-yl]acetic acid (from Step 5) in DMF was treated with dimethylamine hydrochloride (1.05 eq.) and HATU (1.05 eq.). The solution was cooled to 0° C. then treated with DIEA (4 eq.) then stirred for 12 h at 20° C. The mixture was diluted with AcOEt then washed sequentially with aqueous HCl (1 N), saturated aqueous NaHCO₃ and brine. The dried organic layer was concentrated and the residue was purified by flash chromatography on silica gel (5:95 AcOEt/petroleum ether) to afford the title compound (90%) as a solid.

¹H NMR (400 MHz, DMSO-d₆, 300 K) δ 1.35-1.50 (m, 3H), 1.68-1.75 (m, 3H), 1.80-2.00 (m, 4H), 2.82-2.88 (m, 1H), 2.87 (s, 3H), 3.17 (s, 3H), 3.86 (s, 3H,), 5.26 (s, 2H), 7.65 (d, J 8.4 Hz, 1H), 7.81 (d, J 8.4 Hz, 1H), 8.07 (s, 1H); MS (ES⁺) m/z 421 (M+H)⁺.

Step 7: 3-cyclohexyl-1-[2-(dimethylamino)-2-oxoethyl]-2-(4-methylphenyl)-1H-indole-6-carboxylic acid

A solution (0.1 M) of methyl 2-bromo-3-cyclohexyl-1-[2-(dimethylamino)-2-oxoethyl]-1H-indole-6-carboxylate (from Step 6) in DME and EtOH (5:2) was treated with 4-methylphenylboronic acid (1.2 eq.). Aqueous Na₂CO₃ (2 N, 8.5 eq.) was added and the solution was degassed, then treated with Pd(PPh₃)₄ (0.1 eq.). The mixture was heated at 80° C. for 4 h, then cooled and diluted with AcOEt and brine. The organic phase was separated and dried then concentrated under reduced pressure. The residue was purified by filtration through silica gel (1:9 AcOEt/petroleum ether) to give a solid that was dissolved in CH₂Cl₂. The resulting solution (0.1 M) was treated dropwise with BBr₃ (3 eq.) then stirred at 20° C. for 2 h. The mixture was concentrated under reduced pressure and the residue was treated with aqueous HCl (1 N) then filtered. Purification by HPLC (stationary phase: Waters Symmetry C₁₈19 mm×100 mm) gave the title compound (66%) as a solid.

¹H NMR (300 MHz, DMSO-d₆, 300 K) δ 1.09-1.42 (m, 3H), 1.59-1.99 (m, 7H), 2.41 (s, 3H), 2.48-2.65 (m, 1H), 2.83 (s, 3H), 2.93 (s, 3H), 4.86 (s, 2H), 7.21 (d, J 7.3 Hz, 2H), 7.35 (d, J 7.3 Hz, 2H), 7.66 (d, J 8.4 Hz, 1H), 7.83 (d, J 8.4 Hz, 1H), 7.92 (s, 1H), 12.60 (br s, 1H); MS (ES⁺) m/z 419 (M+H)⁺.

### EXAMPLE 2

### 3-cyclohexyl-1-[2-(dimethylamino)-2-oxoethyl]-2-(2-fluorophenyl)-1H-indole-6-carboxylic acid

Following the procedure described above for Example 1, Step 7, treatment of methyl 2-bromo-3-cyclohexyl-1-[2-(dimethylamino)-2-oxoethyl]-1H-indole-6-carboxylate (from Example 1, Step 6) with 2-fluorophenylboronic acid gave a residue that was purified by HPLC (stationary phase: Waters Symmetry C₁₈ 19 mm×100 mm) to afford the title compound (53%) as a solid.

¹H NMR (300 MHz, DMSO-d₆, 300 K) δ 1.10-1.40 (m, 3H), 1.60-1.90 (m, 7H), 2.39-2.62 (m, 1H), 2.77 (s, 3H), 2.91 (s, 3H), 4.62 (d, J 17.5 Hz, 1H), 5.15 (d, J 17.5 Hz, 1H), 7.26-7.48 (m, 3H), 7.54-7.64 (m, 1H), 7.68 (d, J 8.4 Hz, 1H), 7.85 (d, J 8.4 Hz, 1H), 8.00 (s, 1H); MS (ES⁺) m/z 423 (M+H)⁺.

### EXAMPLE 3

### 3-cyclohexyl-1-[2-(dimethylamino)-2-oxoethyl]-2-(3-methylphenyl)-1H-indole-6-carboxylic acid

Following the procedure described above for Example 1, Step 7, treatment of methyl 2-bromo-3-cyclohexyl-1-[2-(dimethylamino)-2-oxoethyl]-1H-indole-6-carboxylate (from Example 1, Step 6) with 3-methylphenylboronic acid gave a residue that was purified by HPLC (stationary phase: Waters Symmetry C₁₈ 19 mm×100 mm) to afford the tide compound (61%) as a solid.

¹H NMR (300 MHz, DMSO-d₆, 300 K) δ 1.11-1.41 (m, 3H), 1.60-2.00 (m, 7H), 2.39 (s, 3H), 2.48-2.66 (m, 1H), 2.83 (s, 3H), 2.91 (s, 3H), 4.86 (s, 2H), 7.07-7.20 (m, 2H), 7.32 (d, J 7.3 Hz, 1H), 7.43 (t, J 7.3 Hz, 1H), 7.66 (d, J 8.4 Hz, 1H), 7.84 (d, J 8.4 Hz, 1H), 7.94 (s, 1H), 12.60 (s, 1H); MS (ES⁺) m/z 419 (M+H)⁺.

### EXAMPLE 4

### 3-cyclohexyl-1-[2-(dimethylamino)-2-oxoethyl]-2-(2-hydroxypyrimidin-5-yl)-1H-indole-carboxylic acid

Following the procedure described above for Example 1, Step 7, treatment of methyl 2-bromo-3-cyclohexyl-1-[2-(dimethylamino)-2-oxoethyl]-1H-indole-6-carboxylate (from Example 1, Step 6) with 2-methoxypyrimidin-5-ylboronic acid gave a residue that was purified by HPLC (stationary phase: Waters Symmetry C₁₈ 19 mm×100 mm; mobile phase: linear gradient from 20% to 100% MeCN (containing 0.1% TFA) in H₂O (containing 0.1% TFA) over 10 min) to afford the title compound (21%) as a solid.

¹H NMR (400 MHz, DMSO-d₆, 300 K) δ 1.22-1.40 (m, 3H), 1.62-1.90 (m, 7H), 2.45-2.62 (m, 1H), 2.83 (s, 3H), 3.06 (s, 3H), 5.05 (s, 2H), 7.65 (d, J 8.4 Hz, 1H), 7.81 (d, J 8.4 Hz, 1H), 7.98 (s, 1H), 8.16 (s, 2H); MS (ES⁺) m/z 423 (M+H)⁺.

### EXAMPLE 5

### 3-cyclohexyl-1-[2-(dimethylamino)-2-oxoethyl]-2-(3-furyl)-1H-indole-6-carboxylic acid

Following the procedure described above for Example 1, Step 7, treatment of methyl 2-bromo-3-cyclohexyl-1-[2-(dimethylamino)-2-oxoethyl]-1H-indole-6-carboxylate (from Example 1, Step 6) with 3-furylboronic acid gave a residue that was purified by HPLC (stationary phase: Waters Symmetry C₁₈ 19 mm×100 mm; mobile phase: linear gradient from 20% to 100% MeCN (containing 0.1% ThA) in H₂O (containing 0.1% TFA) over 11 min) to afford the title compound (25%) as a solid.

¹H NMR (400 MHz, DMSO-d₆, 300 K) δ 1.20-1.42 (m, 3H), 1.63-1.95 (m, 7H), 2.65-2.78 (m, 1H), 2.85 (s, 3H), 3.03 (s, 3H), 4.99 (s, 2H), 6.50 (s, 1H), 7.63 (d, J 8.4 Hz, 1H), 7.78 (s, 1H), 7.80 (d, J 8.4 Hz, 1H), 7.87 (s, 1H), 7.95 (s, 1H); MS (ES⁺) m/z 395 (M+H)⁺.

### EXAMPLE 6

### 3-[6-carboxy-3-cyclohexyl-1-[2-(dimethylamino)-2-oxoethyl]-1H-indol-2-yl}pyridinium trifluoroacetate

Following the procedure described above for Example 1, Step 7, treatment of methyl 2-bromo-3-cyclohexyl-1-[2-(dimethylamino)-2-oxoethyl]-1H-indole-6carboxylate (from Example 1, Step 6) with 3-pyridyl boronic acid gave a residue that was purified by HPLC (stationary phase: Waters Symmetry C₁₈ 19 mm×100 mm; mobile phase: linear gradient from 20% to 100% MeCN (containing 0.1% TFA) in H₂O (containing 0.1% TFA) over 10 min) to afford the title compound (23%) as a solid.

¹H NMR (400 MHz, DMSO-d₆, 300 K) δ 1.15-1.40 (m, 3H), 1.62-1.92 (m, 7H), 2.45-2.58 (m, 1H), 2.79 (s, 3H), 2.95 (s, 3H), 4.96 (s, 2H), 7.68 (d, J 8.4 Hz, 1H), 7.73 (dd, J 7.6, 4.8 Hz, 1H), 7.87 (d, J 8.4 Hz, 1H), 7.94 (d, J 7.6 Hz, 1H), 8.01 (s, 1H), 8.62 (s, 1H), 8.78 (d, J 4.8 Hz, 1H); MS (ES⁺) m/z 406 (M+H)⁺.

### EXAMPLE 7

### 3-cyclohexyl-1-[2-(dimethylamino)-2-ozoethyl]-2-phenyl-1H-indole-6-carboxylic acid

Step 1: methyl 3-amino-4-hydroxybenzoate

A solution (0.2 M) of acetyl chloride (3.0 eq.) in MeOH was prepared at 0° C. then allowed to warm to 20° C. 3-amino-4-hydroxybenzoic acid (1.0 eq.) was added and the mixture was heated under reflux for 12 h then cooled and concentrated in vacuo. The residue was triturated with H₂O and dried to afford the title compound (99%) as a solid.

¹H NMR (300 MHz, DMSO-d₆, 300 K) δ 3.83 (s, 3H), 7.15 (d, J 8.5 Hz, 1H), 7.79 (dd, J 2.1, 8.5 Hz, 1H), 7.93 (d, J 2.1 Hz, 1H), 11.65 (br s, 1H).

Step 2: methyl 4-hydroxy-3-[(trifluoroacetyl)amino]benzoate

A solution (0.2 M) of methyl 3-amino-4-hydroxybenzoate (form Step 1) in THF was cooled to 0° C. and treated dropwise with trifluoroacetic anhydride (2.0 eq.). The mixture was stirred at 0° C. for 2 h then at 20° C. for 1 h. The pH was adjusted to 7.5 by addition of saturated aqueous NaHCO₃ and the solution was extracted with AcOEt. The organic layer was washed with brine and dried, then concentrated to afford the title compound (87%) as a solid.

¹H NMR (400 MHz, DMSO-d₆, 300 K) δ 3.82 (s, 3H), 7.02 (d, J 8.5 Hz, 1H), 7.77 (dd, J 2.1, 8.5 Hz, 1H), 7.97 (d, J 2.1 Hz, 1H), 10.82 (br s, 1H).

Step 3: methyl 3-[(trifluoroacetyl)amino]-4-{[(trifluoromethyl)sulfonyl]oxy}benzoate

A solution (0.8 M) of methyl 4hydroxy-3-[(trifluoroacetyl)amino]benzoate (from Step 3) in dry pyridine was cooled to 0° C. and treated dropwise with trifluoromethanesulfonyl anhydride (1.15 eq.). The mixture stirred for 1 h at 20° C. then diluted with H₂O and AcOEt. The organic layer was separated and washed with aqueous HCl (1 N) and brine then dried. Removal of the solvent afforded a residue that was purified by flash chromatography (1:9 AcOEt/petroleum ether eluent) to afford the title compound (64%) as a solid.

¹H NMR (300 MHz, DMSO-d₆, 300 K) δ 3.92 (s, 3H), 7.82 (d, J 8.7 Hz, 1H), 8.11 (dd, J 2.2, 8.7 Hz, 1H), 8.17 (d, J 2.2 Hz, 1H), 11.81 (s, 1H).

Step 4: methyl 2-phenyl-1H-indole-6-carboxylate

A solution (0.3 M) of methyl 3-[(trifluoroacetyl)amino]-4-{[(trifluoromethyl)sulfonyl]oxy}benzoate (from Step 3) in dry DMF was treated with ethynyl benzene (2.0 eq.), tetramethyl guanidine (10.0 eq.), PdCl₂(PPh₃)₂ (0.1 eq.) and Cul (0.1 eq.). The mixture was stirred at 20° C. for 1 h then heated at 100° C. for 8 h. The cooled solution was diluted with Et₂O and filtered through Celite™. The filtrate was washed with aqueous HCl (1 N) and brine then dried. Removal of the solvent afforded a residue that was purified by flash chromatography (1:9 AcOEt/petroleum ether eluent) to afford the title compound (39%) as a solid.

¹H NMR (400 MHz, DMSO-d₆, 300 K) δ 3.88 (s, 3H), 7.04 (s, 1H), 7.40 (t, J 7.6 Hz, 1H), 7.53 (t, J 7.6 Hz, 2H), 7.65 (s, 2H), 7.92 (d, J 7.6 Hz, 2H), 8.08 (s, 1H), 11.94 (s, 1H).

Step 5: methyl 3-cyclohex-2-en-1-yl-2-phenyl-1H-indole-6-carboxylate

A solution (0.06 M) of methyl 2-phenyl-1H-indole-6-carboxylate (from Step 4) in dry DMF was cooled to 0° C. and treated with NaH (1.1 eq.). The mixture was warmed to 20° C. and stirred for 0.5 h, then cooled to 0° C. A solution (0.3 M) 3-bromocyclohexene (1.3 eq.) in DMP was added dropwise and the mixture was stirred for 2 h at 20° C. Aqueous HCl (1 N) and AcOEt were added and the organic layer was separated, washed with brine and dried. Removal of the solvent afforded a residue that was purified by flash chromatography on silica gel (1:9 AcOEt/petroleum ether) to afford the title compound (79%) as a solid.

¹H NMR (400 MHz, DMSO-d₆, 300 K) δ 1.57-1.74 (m, 1H), 1.82-2.05 (m, 3H), 2.06-2.18 (m, 1H), 2.18-2.32 (m, 1H), 3.67-3.81 (m, 1H), 3.87 (s, 3H), 5.69 (d, J 10.4 Hz, 1H), 5.82-5.92 (m, 1H), 7.44-7.52 (m, 1H), 7.54-7.63 (m, 5H), 7.68 (d, J 8.4 Hz, 1H), 8.03 (s, 1H), 11.59 (s, 1H).

Step 6: methyl 3-cyclohexyl-2-phenyl-1H-indole-6-carboxylate

A solution (0.01 M) of methyl 3-cyclohex-2-en-1-yl-2-phenyl-1H-indole-6-carboxylate (from Step 5) in MeOH was treated with 10% Pd/C (10% wt.). The resulting suspension was stirred for 12 h under an atmosphere of hydrogen then purged with nitrogen and filtered. The filtrate was concentrated to afford the title compound (91%) as a solid.

¹H NMR (300 MHz, DMSO-d₆, 300 K) δ 1.21-1.45 (m, 3H), 1.67-1.90 (m, 5H), 1.91-2.11 (m, 2H), 2.82-2.99 (m, 1H), 3.88 (s, 3H), 7.43-7.52 (m, 1H), 7.54-7.60 (m, 4H), 7.62 (dd, J 1.4, 8.4 Hz, 1H), 7.87 (d, J 8.4 Hz, 1H), 8.02 (d, J 1.4 Hz, 1H), 11.51 (s, 1H).

Step 7: methyl 1-(2-tert-butoxy-2-oxoethyl)-3-cyclohexyl-2-phenyl-1H-indole-6-carboxylate

A solution (0.05 M) of methyl 3-cyclohexyl-2-phenyl-1H-indole-6-carboxylate (from Step 6) in DMF was treated with NaH (1.4 eq.) then stirred for 0.5 h. tert-Butyl bromoacetate (2.0 eq.) was added dropwise, and the mixture was heated at 50° C. for 12 h. After cooling to room temperature the solution was diluted with AcOEt and washed sequentially with aqueous HCl (1 N) and brine. The dried organic phase was concentrated to give a residue that was purified by flash chromatography on silica gel (5:95 AcOEt/petroleum ether) to afford the title compound (83%) as a solid.

¹H NMR (300 MHz, DMSO-d₆, 300 K) δ 1.19-1.27 (m, 3H), 1.32 (s, 9H), 1.62-1.95 (m, 7H), 2.59-2.67 (m, 1H), 3.89 (s, 3H), 4.75 (s, 2H), 7.33-7.37 (m, 2H), 7.54-7.57 (m, 3H), 7.71 (d, J 8.4 Hz, 1H), 7.89 (d, J 8.4 Hz, 1H), 8.09 (s, 1H).

Step 8: [3-cyclohexyl-6-(methoxycarbonyl)-2-phenyl-1H-indol-1-yl]acetic acid

A solution (0.07 M) of methyl 1-(2-tert-butoxy-2-oxoethyl)-3-cyclohexyl-2-phenyl-1H-indole-6carboxylate (from Step 7) in a 1:1 (v/v) mixture of CH₂Cl₂/TFA was stirred for 4 h then concentrated under reduced pressure. The residue was triturated with Et₂O to afford the tide compound (98%) as a solid.

¹H NMR (400 MHz, DMSO-d₆, 300 K) δ 1.17-1.29 (m, 3H), 1.63-1.75 (m, 5H), 1.68-1.90 (m, 2H), 2.51-2.60 (m, 1H), 3.86 (s, 3H), 4.73 (s, 2H), 7.33 (d, J 8.0 Hz, 2H), 7.51-7.56 (m, 3H), 7.68 (d, J 8.4 Hz, 1H), 7.86 (d, J 8.4 Hz, 1H), 8.02 (s, 1), 12.96 (br s, 1H).

Step 9: 3-cyclohexyl-1-[2-(dimethylamino)-2-oxoethyl]-2-phenyl-1H-indole-6-carboxylic acid

A solution (0.04 M) of [3-cyclohexyl-6-(methoxycarbonyl)-2-phenyl-1H-indol-1-yl]acetic acid (from Step 8) in DMF was treated with dimethylamine hydrochloride (1.0 eq.) and HATU (1.0 eq.). DIEA (3.0 eq.) was added and the mixture was stirred for 12 h. The mixture was diluted with AcOEt then washed sequentially with aqueous HCl (1 N), saturated aqueous NaHCO₃ and brine. The dried organic layer was concentrated and diluted to with CH₂Cl₂. The resulting solution (0.03 M) was treated dropwise with BBr₃ (3 eq.) then stirred for 2 h. The solvent was removed under reduced pressure and the residue was treated with aqueous HCl (1 N) then filtered. Purification by HPLC (stationary phase: Waters Symmetry C₁₈ 19 mm×100 mm; mobile phase: linear gradient from 40% to 100% MeCN (containing 0.1% TFA) in H₂O (containing 0.1% TFA) over 11 min) gave the tide compound (70%) as a solid.

¹H NMR (400 MHz, DMSO-d₆, 300 K) δ 1.13-1.30 (m, 3H), 1.63-1.75 (m, 5H), 1.80-1.90 (m, 2H), 2.53-2.59 (m, 1H), 2.79 (s, 3H), 2.89 (s, 3H), 4.84 (s, 2H), 7.31 (d, J 6.4 Hz, 2H), 7.48-7.53 (m, 3H), 7.64 (d, J 8.4 Hz, 1H), 7.81 (d, J 8.4 Hz, 1H), 7.92 (s, 1H); MS (ES⁺) m/z 405 (M+H)⁺.

### EXAMPLE 8

### 3-cyclohexyl-1-[2-(methylamino)-2-oxoethyl]-2-phenyl-1H-indole-6-carboxylic acid

Following the procedure described above for Example 7, Step 9, treatment of [3-cyclohexyl-6-(methoxycarbonyl)-2-phenyl-1H-indol-1-yl]acetic acid (from Example 7, Step 8) with methylamine hydrochloride gave a residue that was purified by SPE (stationary phase: Isolute C₁₈ 20 g; mobile phase: 10% to 60% MeCN in H₂O) to afford the title compound (51%) as a solid.

¹H NMR (400 MHz, DMSO-d₆, 300 K) δ 1.16-1.30 (m, 3H), 1.63-1.73 (m, 5H), 1.80-1.89 (m, 2H), 2.51-2.55 (m, 1H), 2.58 (d, J 4.4 Hz, 3H), 4.51 (s, 2H), 7.38 (d, 6.4 Hz, 2H), 7.48-7.52 (m, 3H), 7.66 (d, J 8.4 Hz, 1H), 7.8 (d, J 8.4 Hz, 1H), 7.88 (s, 1H), 7.98 (d, J 4.4 Hz, 1H); MS (ES⁺) m/z 391 (M+H)⁺.

### EXAMPLE 9

### 3-cyclohexyl-1-(2-morpholin-4-yl-2-ozoethyl)-2-phenyl-1H-indole-6-carboxylic acid

Following the procedure described above for Example 7, Step 9, treatment of [3-cyclohexyl-6-(methoxycarbonyl)-2-phenyl-1H-indol-1-yl]acetic acid (from Example 7, Step 8) with morpholine (1.2 eq.) gave a residue that was purified by HPLC (stationary phase: Waters Symmetry C₁₈ 19 mm×100 mm; mobile phase: linear gradient from 30% to 100% MeCN (containing 0.1% TFA) in H₂O (containing 0.1% TFA) over 10 min) to afford the title compound (66%) as a solid.

¹H NMR (400 MHz, DMSO-d₆, 300 K) δ 1.17-1.30 (m, 3H), 1.63-1.77 (m, 5H), 1.80-1.90 (m, 2H), 2.53-2.58 (m, 1H), 3.31-3.39 (im, 8H), 4.89 (s, 2H), 7.31 (d, J 8.0 Hz, 2H), 7.49-7.54 (m, 3H), 7.65 (d, J 8.4 Hz, 1H), 7.82 (d, J 8.4 Hz, 1H), 7.96 (s, 1H); MS (ES⁺) m/z 447 (M+H)⁺.

### EXAMPLE 10

### 3-cyclohexyl-1-(2-{[(1-methylpyrrolidin-3-yl)methyl]amino}-2-oxoethyl)-2-phenyl-1H-indole-6-carboxylic acid hydrochloride

Following the procedure described above for Example 7, Step 9, treatment of [3-cyclohexyl-6-(methoxycarbonyl)-2-phenyl-1H-indol-1-yl]acetic acid (from Example 7, Step 8) with 1-(1-methylpyrrolidin-3-yl)methanamine (1.2 eq.) gave a residue that was purified by SPE (stationary phase: Isolute C₁₈ 20 g; mobile phase: 10% to 70% MeCN in H₂O) to afford the title compound (47%) as a solid.

¹H NMR (400 MHz, DMSO-d₆, 300 K) δ 1.17-1.30 (m, 4H), 1.41-1.50 (m, 1H), 1.63-1.78 (m, 5H), 1.82-1.91 (m, 3H), 2.30-2.39 (mn, 1H), 2.45 (s, 3H), 2.53-2.58 (m, 1H), 2.67-2.90 (m, 3H), 3.07-3.09 (m, 2H), 4.54 (s, 2H), 7.39 (d, J 8.0 Hz, 2H), 7.49-7.54 (m, 3H), 7.66 (d, J 8.4 Hz, 3H), 7.83 (d, J 8.4 Hz, 3H), 7.93 (s, 1H), 8.25 (t, J 6.0 Hz, 1H); MS (ES⁺) m/z 474 (M+H)⁺.

### EXAMPLE 11

### 3-cyclohexyl-1-[2-(4-methylpiperazin-1-yl)-2-oxoethyl]-2-phenyl-1H-indole-6-carboxylic acid trifluoroacetate

Following the procedure described above for Example 7, Step 9, treatment of [3-cyclohexyl-6-(methoxycarbonyl)-2-phenyl-1H-indol-1-yl]acetic acid (from Example 7, Step 8) with 1-methylpiperazine (1.2 eq.) gave a residue that was purified by HPLC (stationary phase: Waters Symmetry C₁₈ 19 mm×100 mm; mobile phase: linear gradient from 10% to 100% MeCN (containing 0.1% TFA) in H₂O (containing 0.1% TFA) over 10 min) to afford the title compound (38%) as a solid.

¹H NMR (400 MHz, DMSO-d₆, 300 K) δ 1.17-1.35 (m, 3H), 1.63-1.73 (m, 5H), 1.80-1.89 (m, 2H), 2.53-2.60 (m, 1H), 2.81 (s, 3H), 2.70-2.97 (m, 4H), 3.18-3.42 (m, 2H), 3.97-4.11 (m, 1H), 4.31-4.40 (m, 1H), 4.88-5.12 (m, 2H), 7.30 (d, J6.8 Hz, 2H), 7.50-7.55 (m, 3H), 7.66 (d, J 8.4 Hz, 1H), 7.83 (d, J 8.4 Hz, 1H), 7.98 (s, 1H), 9.88 (br s, 1H), 12.50 (br s, 1H); MS (ES⁺) m/z 460 (M+H)⁺.

### EXAMPLE 12

### 3-cyclohexyl-1-(2-{[1-(5-methyl-4H-1,2,4-triazol-3-yl)ethyl]amino}-2-oxoethyl)-2-phenyl-1H-indole-6-carboxylic acid trifluoroacetate

Following the procedure described above for Example 7, Step 9, treatment of [3-cyclohexyl-6-(methoxycarbonyl)-2-phenyl-1H-indol-1-yl]acetic acid (from Example 7, Step 8) with 1-(5-methyl-4H-1,2,4-triazol-3-yl)ethanamine dihydrochloride (1.2 eq.) and DIEA (5.0 eq.) gave a residue that was purified by HPLC (stationary phase: Waters Symmetry C₁₈ 19 mm×100 mm; mobile phase: linear gradient from 10% to 90% MeCN (containing 0.1% TFA) in H₂O (containing 0.1% TFA) over 10 min) to afford the title compound (46%) as a solid.

¹H NMR (400 MHz, DMSO-d₆, 300 K) δ 1.15-1.30 (m, 3H), 1.36 (d, J 6.8 Hz, 3H), 1.63-1.73 (m, 5H), 1.82-1.90 (m, 2H), 2.34 (s, 3H), 2.53-2.60 (m, 1H), 4.57-4.63 (m, 2H), 4.94-4.98 (m, 1H), 7.36 (d, J 6.4 Hz, 1H), 7.47-7.51 (m, 3H), 7.65 (d, J 8.4 Hz, 3H), 7.82 (d, J 8.4 Hz, 1H), 7.97 (s, 1H), 8.61 (d, J 8.0 Hz, 1H); MS (ES⁺) m/z 486 (M+H)⁺.

### EXAMPLE 13

### 3-cyclohexyl-1-(2-{methyl[(1-methylpiperidin-3-yl)methyl]amino}-2-oxoethyl)-2-phenyl-1H-indole-6-carboxylic acid trifluoroacetate

Following the procedure described above for Example 7, Step 9, treatment of a solution (0.03 M) of [3-yclohexyl-6-(methoxycarbonyl)-2-phenyl-1H-indol-1-yl]acetic acid (from Example 7, Step 8) in CH₂Cl₂ with N-methyl-1-(1-methylpiperidin-3-yl)methanamine (1.2 eq.), HATU (2.0 eq.) and DIEA (6.0 eq.) gave a residue that was purified by HPLC (stationary phase: Waters Symmetry C₁₈ 19 mm ×50 mm; mobile phase: linear gradient from 10% to 90% MeCN (containing 0.1% TFA) in H₂O (containing 0.1% TFA) over 5.5 min) to afford the tide compound (51%) as a solid.

¹H NMR (300 MHz, DMSO-d₆, 340 K) δ 1.13-1.41 (m, 3H), 1.47-1.97 (m, 11H), 1.97-2.19 (m, 1H), 2.57-2.71 (m, 1H), 2.78 (s, 311), 2.94 (s, 3H), 3.04-3.36 (m, 6H), 4.92 (s, 2H), 7.33-7.50 (m, 2H), 7.53-7.65 (m, 3H), 7.73 (d, J 8.2 Hz, 1H), 7.86 (d, J 8.2 Hz, 1H), 8.00 (br s, 1H); MS (ES⁺) m/z 502 (M+H)⁺.

### EXAMPLE 14

### 3-cyclohexyl-1-(2-{[(1-methylpiperidin-3-yl)methyl]amino)-2-oxoethyl)-2-phenyl-1H-indole-6-carboxylic acid trifluoroacetate

Following the procedure described above for Example 7, Step 9, treatment of a solution (0.03 M) of [3-cyclohexyl-6-(methoxycarbonyl)-2-phenyl-1H-indol-1-yl]acetic acid (from Example 7, Step 8) in CH₂Cl₂ with 1-(1-methylpiperidin-3-yl)methanamine (1.2 eq.), HATU (2.0 eq.) and DIEA (6.0 eq.) gave a residue that was purified by HPLC (stationary phase: Waters Symmetry C₁₈ 19 mm×50 mm; mobile phase: linear gradient from 10% to 90% MeCN (containing 0.1% TFA) in H₂O (containing 0.1% TFA) over 5.5 min) to afford the title compound (57%) as a solid.

¹H NMR (300 MHz, DMSO-d₆, 340 K) δ 0.98-1.40 (m, 5H), 1.48-2.04 (m, 12H), 2.57-2.70 (m, 1H), 2.84 (s, 3H), 2.97-3.11 (m, 2H), 3.17-3.50 (m, 2H), 4.59 (s, 2H), 7.39-7.49 (m, 2H), 7.50-7.61 (m, 3H), 7.71 (d, J 8.4 Hz, 11), 7.84 (d, J 8.4 Hz, 1H), 7.99 (s, 1H), 8.06 (t, J 5.0 Hz, 1H), 9.15 (br s, 1H); MS (ES⁺) m/z 488 (M+H)⁺.

### EXAMPLE 15

### 3-cyclohexyl-1-(2-{methyl[(1-methylpiperidin-2-yl)methyl]amino}-2-oxoethyl)-2-phenyl-1H-indole-6-carboxylic acid trifluoroacetate

Following the procedure described above for Example 7, Step 9, treatment of a solution (0.03 M) of [3-cyclohexyl-6-(methoxycarbonyl)-2-phenyl-1H-indol-1-yl]acetic acid (from Example 7, Step 8) in CH₂Cl₂ with N-methyl-1-(1-methylpiperidin-2-yl)methanamine (1.2 eq.), HATU (2.0 eq.) and DIEA (6.0 eq.) gave a residue that was purified by HPLC (stationary phase: Waters Symmetry C₁₈ 19 mm ×50 mm; mobile phase: linear gradient from 10% to 90% MeCN (containing 0.1% TFA) in H₂O (containing 0.1% TEA) over 5.5 min) to afford the title compound (57%) as a solid.

¹H NMR (300 MHz, DMSO-d₆, 340 K) δ 1.12-1.42 (m, 5H), 1.54-1.98 (m, 12H), 2.57-2.70 (m, 1H), 2.79 (s, 3H), 2.96 (s, 3H), 3.11-3.83 (m, 4H), 4.93 (s, 2H), 7.30-7.45 (m, 2H), 7.47-7.61 (m, 3H), 7.69 (d, J 8.4 Hz, 1H), 7.83 (d, J 8.4 Hz, 1H), 7.98 (s, 1H); MS (ES⁺) m/z 502 (M+H)⁺.

### EXAMPLE 16

### 3-cyclohexyl-1-(2-{methyl[(5-methyl-1H-imidazol-2-yl)methyl]amino}-2-oxoethyl)-2-phenyl-1H-indole-6-carboxylic acid trifluoroacetate

Following the procedure described above for Example 7, Step 9, treatment of a solution (0.03 M) of [3-cyclohexyl-6-(methoxycarbonyl)-2-phenyl-1H-indol-1-yl]acetic acid (from Example 7, Step 8) in CH₂Cl₂ with N-methyl-1-(5-methyl-1H-imidazol-2-yl)methanamine (1.2 eq.), HATU (2.0 eq.) and DIEA (6.0 eq.) gave a residue that was purified by HPLC (stationary phase: Waters Symmetry C₁₈ 19 mm×50 mm; mobile phase: linear gradient from 10% to 90% MeCN (containing 0.1% TPA) in H₂O (containing 0.1% TFA) over 5.5 min) to afford the title compound (65%) as a solid.

1H NMR (300 MHz, DMSO-d₆, 340 K) δ 1.08-1.39 (m, 3H), 1.55-1.99 (m, 7H), 2.28 (s, 3H), 2.52-2.68 (m, 1H), 3.02 (s, 3H), 4.62 (s, 2H), 4.93 (s, 2H), 7.11-7.39 (m, 3H), 7.41-7.60 (m, 3H), 7.69 (d, J 8.4 Hz, 1H), 7.81 (d, J 8.4 Hz, 1H), 7.99 (s, 1H); MS (ES⁺) m/z 485 (M+H)⁺.

### EXAMPLE 17

### 3-cyclohexyl-1-(2-{([2-(diethylamino)ethyl]amino}-2-oxoethyl)-2-phenyl-1H-indole-6-carboxylic acid trifluoroacetate

Following the procedure described above for Example 7, Step 9, treatment of a solution (0.03 M) of (3-cyclohexyl-6-(methoxycarbonyl)-2-phenyl-1H-indol-1-yl]acetic acid (from Example 7, Step 8) in CH₂Cl₂ with N,N-dimethylethane-1,2-diamine (1.2 eq.), HATU (2.0 eq.) and DIEA (6.0 eq.) gave a residue that was purified by HPLC (stationary phase: Waters Symmetry C₁₈ 19 mm×50 mm; mobile phase: linear gradient from 10% to 90% MeCN (containing 0.1% TFA) in H₂O (containing 0.1% TFA) over 5.5 min) to afford the title compound (63%) as a solid.

¹H NMR (300 MHz, DMSO-d₆, 300 K) δ 1.06-1.45 (m, 3H), 1.56-2.03 (m, 7H), 2.51-2.65 (m, 1H), 2.80 (d, J4.6 Hz, 6H), 3.04-3.19 (m, 2H), 3.35-3.49 (m, 2H), 4.63 (s, 2H), 7.33-7.45 (m, 2H), 7.61-7.48 (m, 3H), 7.69 (d, J 8.4 Hz, 1H), 7.86 (d, J 8.4 Hz, 1H), 7.97 (s, 1H), 8.38 (t, J 5.4 Hz, 1H), 9.38 (br s, 1H), 12.60 (br s, 1H); MS (ES⁺) m/z 448 (M+H)⁺.

### EXAMPLE 18

### 3-cyclohexyl-1-(2-{[2-(1-methylpyrrolidin-3-yl)ethyl]amino}-2-oxoethyl)-2-phenyl-1H-indole-6-carboxylic acid trifluoroacetate

Following the procedure described above for Example 7, Step 9, treatment of a solution (0.03 M) of [3-cyclohexyl-6-(methoxycarbonyl)-2-phenyl-1H-indol-1-yl)acetic acid (from Example 7, Step 8) in CH₂Cl₂ with 2-(1-methylpyrrolidin-3-yl)ethanamine (1.2 eq.), HATU (2.0 eq.) and DIEA (6.0 eq.) gave a residue that was purified by HPLC (stationary phase: Waters Symmetry C₁₈ 19 mm×50 mm; mobile phase: linear gradient from 10% to 90% MeCN (containing 0.1% TFA) in H₂O (containing 0.1% TFA) over 5.5 min) to afford the title compound (64%) as a solid.

¹H NMR (300 MHz, DMSO-d₆, 300 K) δ 1.04-1.45 (m, 3H), 1.48-2.13 (m, 13H), 2.14-2.35 (m, 1H), 2.54-2.70 (m, 1H) 2.80 (d, J4.9 Hz, 3H), 2.97-3.30 (m, 3H), 3.49-3.68 (m, 1H), 4.59 (s, 2H), 7.36-7.48 (m, 2H), 7.50-7.61 (m, 3H), 7.70 (dd, J 8.4, 1.1 Hz, 1H), 7.86 (d, J 8.4 Hz, 1H), 7.96 (d, J 1.1 Hz, 1H), 8.28 (t, J 5.6 Hz, 1H), 9.39 (br s, 1H), 12.64 (br s, 1H); MS (ES⁺) m/z 488 (M+H)⁺.

### EXAMPLE 19

### 2-[3-cyclohexyl-2-phenyl-6-(1H-tetrazol-5-yl)-1H-indol-1-yl]-N,N-dimethylacetamide

Step 1: 3-cyclohexyl-1-[2-(dimethylamino)-2-oxoethyl]-2-phenyl-1H-indole-6-carboxamide

A solution (0.15 M) of 3-cyclohexyl-1-[2-(dimethylamino)-2-oxoethyl]-2-phenyl-1H-indole-6-carboxylic acid (prepared as described in Example 7) in DMF was treated with pyridine (0.67 eq.), NH₄HCO₃ (1.45 eq.) and di-tert-butyl dicarbonate (1.5 eq.). The mixture was stirred for 72 h then diluted with aqueous HCl (1 N) and AcOEt. The organic phase was separated, washed with brine and dried. Removal of the solvent afforded the title compound (67%) as a solid.

¹H NMR (600 MHz, DMSO-d₆, 300 K) δ 1.14-1.36 (m, 3H), 1.61-1.79 (mn, 5H), 1.81-1.93 (m, 2H), 2.51-2.60 (m, 1H), 2.81 (s, 3H), 2.92 (s, 3H), 4.80 (s, 2H), 7.20 (br s, 1H), 7.31 (d, J7.1 Hz, 2H), 7.45-7.54 (m, 3H), 7.60 (s, J 8.5 Hz, 1H), 7.76 (d, J 8.5 Hz, 1H), 7.86 (br s, 1H), 7.87 (s, 1H); MS (ES⁺) m/z 404 (M+H)⁺.

Step 2: 2-(6-cyano-3-cyclohexyl-2-phenyl-1H-indol-1-yl)-N,N-dimethylacetamide

A solution (0.04 M) of 3-cyclohexyl-1-[2-(dimethylamino)-2-oxoethyl]-2-phenyl-1H-indole-6-carboxamide (from Step 1) in CH₂Cl₂ was treated with triethylamine (6.4 eq.) and then cooled to 0° C. Trifluoroacetic anhydride was (3.2 eq.) was added dropwise and the mixture was warmed to 20° C. After 1 h the solvent was removed and the residue was taken up in AcOEt and aqueous HCl (1 N). The organic layer was separated, washed with brine and dried. Removal of the solvent gave a residue that was purified by flash chromatography on silica gel (1:9 AcOEt/petroleum ether) to afford the title compound (90%) as a solid.

¹H NMR (300 MHz, DMSO-d₆, 300 K) δ 1.08-1.40 (m, 3H), 1.58-1.97 (m, 7H, 2.51-2.65 (m, 1H), 2.80 (s, 3H), 2.90 (s, 3H), 4.87 (s, 2H), 7.28-7.36 (m, 2H), 7.38 (dd, J 8.4, 1.2 Hz, 2H), 7.48-7.61 (m, 3H), 7.94 (d, J 8.4 Hz, 1H), 8.00 (d, J 1.2 Hz, 1H); MS (ES⁺) m/z 386 (M+H)⁺.

Step 3: 2-[3-cyclohexyl-2-phenyl-6-(1H-tetraazol-5-yl)-1H-indol-1-yl]-N,N-dimethylacetamide

A solution (0.02 M) of 2-(6-cyano-3-cyclohexyl-2-phenyl-1H-indol-1-yl)-N,N-dimethylacetamide (from Step 2) in toluene was treated with Bu₃SnN₃ (2.0 eq.) and the mixture was heated under reflux for 24 h. The cooled solution was diluted with AcOEt and washed with aqueous HCl (1 N) and then brine. The organic phase was dried and concentrated, and the residue was triturated with pentane to afford a yellow solid. Purification of this material by HPLC (stationary phase: Waters X-terra C₁₈ 19 mm×100 mm) afforded the title compound (45%) as a solid.

¹H NMR (600 MHz, DMSO-d₆, 300 K) δ 1.14-1.27 (m, 2H), 1.27-1.38 (m, 1H), 1.62-1.70 (m, 1H), 1.70-1.81 (m, 4H), 1.83-1.96 (m, 2H), 2.55-2.63 (m, 1H), 2.82 (s, 3H), 2.93 (s, 3H), 4.86 (s, 2H), 7.33 (d, J 6.6 Hz, 2H), 7.38 (dd, J 8.4, 1.2 Hz, 1H), 7.46-7.57 (m, 3H), 7.70 (d, J 8.2 Hz, 1H), 8.97 (d, J 8.2 Hz, 1H), 8.00 (s, 1H); MS (ES⁺) m/z 429 (M+H)⁺.

### EXAMPLE 20

### 3-cyclohexyl-N-methyl-1-(2-morpholin-4-yl-2-ozoethyl)-2-phenyl-1H-indole-6-carboxamide

A solution (0.02 M) of 3-cyclohexyl-1-(2-morpholin4-yl-2-oxoethyl)-2-phenyl-1H-indole-6-carboxylic acid (prepared as described in Example 9) in CH₂Cl₂ was treated with methylamine hydrochloride (1.2 eq.) and HATU (2.0 eq.). DIEA (6.0 eq.) was added and the mixture was stirred for 12 h. The mixture was diluted with CH₂Cl₂ then washed sequentially with aqueous HCl (1 N), aqueous NaOH (1 N) and brine. The dried organic layer was concentrated and the residue was purified by HPLC (stationary phase: Waters Symmetry C₁₈ 19 mm×100 mm) to afford the tide compound (35%) as a solid.

¹H NMR (300 MHz, DMSO-d₆, 300 K) δ 1.20-1.38 (m, 3H), 1.70-1.80 (m, 5H), 1.86-1.98 (m, 2H), 2.34 (s, 3H), 2.58-2.68 (m, 1H), 2.88 (d, J 4.5 Hz, 3H), 3.40-3.54 (m, 6H), 3.55-3.60 (m, 2H), 4.89 (s, 2H), 7.37 (d, J 5.7 Hz, 2H), 7.53-7.62 (m, 4H), 7.84 (d, J 8.4 Hz, 1H), 7.91 (s, 1H), 8.33 (d, J 4.5 Hz, 1H); MS (ES⁺) m/z 460 (M+E)⁺.

### EXAMPLE 21

### 3-cyclohexyl-1-[2-(dimethylamino)-2-oxoethyl]-2-phenyl-1H-pyrrolo[2,3-b]pyridine-6-carboxylic acid

Step 1: (cyclohexylethynyl)(trimethyl)silane

A solution (0.16 M) of 2,2,2-trichloro-1-cyclohexylethyl 4-methylbenzenesulfonate (obtained as described in J. Org. Chem., 65, 1889-1891, 2000) was cooled to −10° C. and a solution of MeLi (1.6 M) was added via dropping funnel keeping the temperature below −5° C. After the addition the temperature was raised to room temperature over 1 h then the mixture was cooled to −78° C. and treated with TMSCl (1.7 eq.). After warming to 0° C. the reaction was quenched with saturated aqueous NH₄Cl solution and Et₂O. The organic layer was separated and washed with brine then dried and concentrated to give a crude material which was submitted to fractional distillation. The title compound (63%) distilled off as colorless liquid at 80-82 ° C./15-17 mbar.

¹H NMR (300 MHz, CDCl₃, 300 K) δ 0.33 (s, 9H), 1.14-1.49 (m, 6H), 1.62-1.82 (m, 4H), 2.30-2.41 (m, 1H).

Step 2: methyl 3cyclohexyl-2-(trinethylsilyl)-1H-pyrrolo[2,3-b]pyridine-6 carboxylate

To a solution (0.1 M) of methyl 6-amino-5-bromo-2-pyridinecarboxylate (obtained as described in J. Org. Chem., 61, 4623-4633, 1996) in DMF were added (cyclohexylethynyl)(trimethyl)silane (obtained as described in step 1) (3 eq.), LiCl (1 eq.), Na₂CO₃ (2 eq.) and Pd(dppf)Cl₂ (0.1 eq.). The suspension was heated at 110° C. for 15 h under argon, then diluted with AcOEt and H₂O and filtered through celiteam. The organics were washed with H₂O and dried, then concentrated and purified by flash chromatography on silica gel (AcOEt/petroleum ether) to afford the title compound (60%) as a pale yellow solid.

¹H NMR (300 MHz, CDCl₃, 300 K) δ 0.39 (s, 9H), 1.39 (m, 3H), 1.82-1.90 (m, 7H), 2.75-2.90 (m, 1H), 4.02 (s, 3H), 7.89 (d, J 8.2 Hz, 1H), 8.13 (d, J 8.2 Hz, 1H), 8.53 (br s, 1H); MS (ES⁺) m/z 331 (M+H)⁺.

Step 3: methyl 2-bromo-1-(2-tert-butoxy-2-oxoethyl)-3-cyclohexyl-1H-pyrrolo[2,3-b]pyridine-6-carboxylate

To a solution (0.15 M) of methyl 3-cyclohexyl-2-(trimethylsilyl)-1H-pyrrolo[2,3-b]pyridine-6-carboxylate (from Step 2) in DMF was added NaH (1.2 eq.) and the suspension was heated at 40° C. for 15 min under nitrogen. To the resulting clear solution tert-butyl bromoacetate (1.3 eq.) was added and the mixture was stirred at 60° C. for 45 min. The reaction was cooled to room temperature, diluted with AcOEt and washed with water, brine, dried and concentrated to give methyl 1-(2-tert-butoxy-2-oxoethyl)-3-cyclohexyl-1H-pyrrolo[2,3-b]pyridine-6-carboxylate as a pale orange solid. MS (ES⁺) mn/z 373 (M+H)⁺.

A solution (0.10 M) of this crude material in CH₂Cl₂ was treated with NBS (1.2 eq.) then stirred at 20° C. for 1 h. The solution was diluted with AcOEt and washed with saturated aqueous Na₂S₂O₃ solution and brine then dried, concentrated and purified by flash chromatography on silica gel (AcOEt/petroleum ether) to afford the title compound (50%) as a white solid.

¹H NMR (400 MHz, CDCl₃, 300 K) δ 1.40-1.46 (m, 2H), 1.47 (s, 9H), 1.81-1.92 (m, 8H), 2.88-2.97 (m, 1H), 4.01 (s, 3H), 5.11 (s, 2H), 7.91 (d, J 8.2 Hz, 1H), 8.09 (d, J 8.2 Hz, 1H); MS (ES⁺) m/z 451 (M+H)⁺.

Step 4: methyl 1-(2-tert-butoxy-2-oxoethyl)-3-cyclohexyl-2-phenyl-1H-pyrrolo[2,3-b]pyridine-6-carboxylate

To a solution (0.08 M) of methyl 2-bromo-1-(2-tert-butoxy-2-oxoethyl)-3-cyclohexyl-1H-pyrrolo[2,3-b]pyridine-6-carboxylate (from Step 3) in toluene were added phenylboronic acid (1.5 eq.), potassium phosphate (1.2 eq.), Pd(PPh₃)₄ (0.5 eq.) and the suspension was heated at 110° C. overnight under argon. After cooling to room temperature, the solvent was removed and the residue dissolved in AcOEt and washed with H₂O, brine, dried, concentrated and purified by flash chromatography on silica gel (AcOEt/petroleum ether) to afford the title compound (60%) as a pale yellow solid.

¹H NMR (400 MHz, CDCl₃, 300 K) δ 1.30-1.33 (m, 2H), 1.33 (s, 9H), 1.79-1.85 (m, 8H), 2.61-2.72 (m, 1H), 4.01 (s, 3H), 4.88 (s, 2H), 7.37-7.51 (m, 5H), 7.95 (d, J 8.4 Hz, 1H), 8.16 (d, J 8.4 Hz, 1H); MS (ES⁺) m/z 449 (M+H)⁺.

Step 5: [3-cyclohexyl-6-(methoxycarbonyl)-2-phenyl-1H-pyrrolo[2,3-b]pyridin-1-yl]acetic acid

A solution (0.06 M) of methyl 1-(2-tert-butoxy-2-oxoethyl)-3-cyclohexyl-2-phenyl-1H-pyrrolo[2,3-b]pyridine-6carboxylate (from Step 4) in CH₂Cl₂/TFA (1:1, v/v) was stirred at room temperature for 1 h. The solvent was removed to afford the title compound (100%) as a yellow solid.

¹H NMR (400 MHz, CDCl₃, 300 K) δ 1.28-1.33 (m, 2H), 1.68-1.82 (n, 8H), 2.61-2.72 (m, 1H), 4.00 (s, 3H), 4.88 (s, 2H), 7.40-7.55 (m, 5H), 7.96 (d, J 8.0 Hz, 1H), 8.21 (d, J 8.0 Hz, 1H); MS (ES⁺) m/z 393 (M+H)⁺.

Step 6: methyl 3-cyclohexyl-1-[2-(dimethylamino)-2-oxoethy]1-2-phenyl-1H-pyrrolo[2,3-b]pyridine-6-carboxylate

To a solution (0.05 M) of [3-cyclohexyl-6-(methoxycarbonyl)-2-phenyl-1H-pyrrolo[2,3-b]pyridin-1-yl]acetic acid (from Step 5) in DMP were added dimethylamine hydrochloride (1.1 eq.), HATU (1.2 eq.), DIEA (3.5 eq.) and the solution was stirred at room temperature under nitrogen for 1.5 h. The solution was diluted with AcOEt and washed with aqueous HCl (1 N), aqueous NaOH (1 N) and brine then dried and concentrated to afford the title compound (100%) as a yellow solid.

¹H NMR (400 MHz, CDCl₃, 300 K) δ1.28-1.33 (m, 2H), 1.72-1.90 (m, 8H), 2.62-2.68 (m, 1H), 2.88 (s, 3H), 3.02 (s, 3H), 4.01 (s, 3H), 4.97 (s, 2H), 7.43-7.51 (m, 5H), 7.93 (d, J 8.2 Hz, 1H), 8.15 (d, J 8.2 Hz, 1H); MS m/z (ES⁺) 420 (M+H)⁺.

Step 7: 3-cyclohexyl-1-[2-(dimethylamino)-2-oxoethyl]-2-phenyl-1H-pyrrolo[2,3-b]pyridine-6-carboxylic acid

To a solution (0.02 M) of methyl 3-cyclohexyl-1-[2-(dimethylamino)-2-oxoethyl]-2-phenyl-1H-pyrrolo[2,3-b]pyridine-6-carboxylate (from Step 6) in CH₂Cl₂ was added neat BBr₃ (3.0 eq.) and the solution was stirred at room temperature under nitrogen for 30 min. The solvent was removed and the residue treated with aqueous HCl (1 N) and purified by preparative HPLC (mobile phase: MeCN/H₂O containing 0.1% TFA) to afford the title compound (45%) as a yellow solid.

¹H NMR (400 MHz, DMSO-d₆, 300 K) δ 1.16-1.35 (m, 3H), 1.63-1.88 (m, 7H), 2.53-2.63 (m, 1H), 2.74 (s, 3H), 2.94 (s, 3H), 4.99 (s, 2H), 7.38-7.58 (m, SH), 7.85 (d, J8.2 Hz, 1H), 8.32 (d, J 8.2 Hz, 1H); MS (ES⁺) m/z 406 (M+H)⁺.

### EXAMPLE 22

### 3-cyclohexyl-1-[2-(4-methylpiperazin-1-yl)-2-oxoethyl]-2-phenyl-1H-pyrrolo[2,3-b]pyridine-5-carboxylic acid

Step 1: methyl 6-amino-5-iodonicotinate

To a solution (0.48 M) of methyl 6-aminonicotinate in glacial acetic acid/TFA (20:1, v/v) was added NIS (1.5 eq.) and the solution was stirred at room temperature overnight. To the solution were added ice, saturated aqueous NH₄OH until pH c. 9 was reached. The precipitate was isolated by filtration, dissolved in CHCl₃ and washed with saturated aqueous Na₂S₂O₃ solution, H₂O and brine then dried and concentrated to afford the title compound (50%) as a solid.

¹H NMR (400 MHz, DMSO-d₆, 300 K) δ 3.77 (s, 3H), 6.90-7.0 (br s, 2H), 8.26 (d, J 2.0 Hz, 1H), 8.49 (d, J 2.0 Hz, 1H); MS (ES⁺) m/z 279 (M+H)⁺.

Step 2: methyl 3-cyclohexyl-2-(trimethylsilyl)-1H-pyrrolo[2,3-b]pyridine-5-carboxylate

To a solution (0.1 M) of methyl 6-amino-5-iodonicotinate (obtained as described in step 1) in DMF were added (cyclohexylethynyl)(trimethyl)silane (from Example 21, Step 1) (3 eq.), LiCl (1 eq.), Na₂CO₃ (2 eq.) and Pd(dppf)Cl₂ (1 eq.). The suspension was heated in microwave for 10 min at 180° C., then diluted with AcOEt/H₂O (1/1, v/v) and filtered through celite™. The organics were washed with brine and dried then concentrated and purified by flash chromatography on silica gel (AcOEt/petroleum ether) to afford the tide compound (20%) as an off-white solid.

¹H NMR (400 MHz, CDCl₃, 300 K) δ 0.39 (s, 9H), 1.37-1.45 (m, 3H), 1.80-1.97 (m, 7H), 2.77-2.85 (m, 1H), 3.97 (s, 3H), 8.71 (s, 1H), 8.93 (s, 1H), 9.04 (br s 1H); MS m/z (ES⁺) 331 (M+H)⁺.

Step 3: methyl 1-(2-tert-butoxy-2-oxoethyl)-3-cyclohexyl-2-(trimethylsilyl)-1H-pyrrolo[2,3-b]pyridine-5-carboxylate

To a solution (0.16 M) of methyl 3-cyclohexyl-2-(trimethylsilyl)-1H-pyrrolo[2,3-b]pyridine-5-carboxylate (from Step 2) in DMF was added NaH (1.2 eq.) and the suspension was heated at 40° C. for 15 min under nitrogen. To the resulting clear solution tert-butyl bromoacetate (1.3 eq.) was added and the mixture stirred at 60° C. for 45 min. After cooling the solution was diluted with AcOEt, washed with H₂O and brine then dried, concentrated and purified by flash chromatography on silica gel (AcOEt/petroleum ether) to afford the title compound (60%) as yellow oil.

¹H NMR (400 MHz, CDCl₃, 300 K) δ 0.39 (s, 9H), 1.37-1.51 (m, 3H), 1.44 (s, 9H), 1.75-1.97 (m, 7H), 2.87-2.98 (m, 1H), 3.95 (s, 3H), 5.08 (s, 2H), 8.66 (d, J 2.0 Hz, 1H), 8.91 (d, J 2.0 Hz, 1H); MS (ES⁺) m/z 447 (M+H)⁺.

Step 4: methyl 2-bromo-1-(2-tert-butoxy-2-oxoethyl)-3-cyclohexyl-1H-pyrrolo[2,3-b]pyridine-5-carboxylate

To a solution (0.1 M) of methyl 1-(2-tert-butoxy-2-oxoethyl)-3-cyclohexyl-2-(trimethylsilyl)-1H-pyrrolo[2,3-b]pyridine-5-carboxylate (from Step 3) in CH₂Cl₂ was added NBS (2 eq.) and the solution stirred at room temperature for 1 h. The solution was diluted with AcOEt and washed with saturated aqueous Na₂S₂O₃ solution and brine then dried, concentrated and purified by flash chromatography on silica gel (AcOEt/petroleum ether) to afford the title compound (45%) as a white solid.

¹H NMR (400 MHz, CDCl₃, 300 K) δ1.37-1.48 (m, 3H), 1.42 (s, 9H), 1.75-1.93 (m, 7H), 2.85-2.96 (m, 1H), 3.97 (s, 3H), 5.02 (s, 2H), 8.62 (d, J 2.0 Hz, 1H), 8.90 (d, J 2.0 Hz, 1H); MS (ES⁺) m/z 451 (M+H)⁺.

Step 5: methyl 1-(2-tert-butoxy-2-oxoethyl)-3-cyclohexyl-2-phenyl-1H-pyrrolo[2,3-b]pyridine-5-carboxylate

To a solution (0.08 M) of methyl 2-bromo-1-(2-tert-butoxy-2-oxoethyl)-3-cyclohexyl-1H-pyrrolo[2,3-b]pyridine-5-carboxylate (from Step 4) in toluene were added phenylboronic acid (1.5 eq.), potassium phosphate (2 eq.), Pd(PPh₃)₄ (0.1 eq.) and the suspension was heated at 110° C. overnight under argon. After cooling, the solvent was removed and the residue was dissolved in AcOEt, washed with H₂O and brine then dried, concentrated and purified by flash chromatography on silica gel (AcOEt/petroleum ether) to afford the title compound (70%) as a colorless oil.

¹H NMR (300 MHz, CDCl₃, 300 K) δ 1.22 (m, 2H), 1.29 (s, 9H), 1.75-1.79 (m, 8H), 2.57-2.63 (m, 1H), 3.95 (s, 3H), 4.71 (s, 2H), 7.31-7.45 (m, 5H), 8.67 (d, J 2.0 Hz, 1H), 8.92 (d, J 2.0 Hz, 1H); MS (ES⁺) m/z 449 (M+H)⁺.

Step 6: 3-cyclohexyl-1-[2-(4methylpiperazin-1-yl)-2-oxoethyl[-2-phenyl-1H-pyrrolo[2,3-b]pyridine-5-carboxylic acid

A solution (0.05 M) of methyl 1-(2-tert-butoxy-2-oxoethyl)-3-cyclohexyl-2-phenyl-1H-pyrrolo[2,3-b]pyridine-5-carboxylate (from Step 5) in CH₂Cl₂/TFA (1:1, v/v) was stirred at room temperature for 1 h. The solvent was removed to give [3-cyclohexyl-5-(methoxycarbonyl)-2-phenyl-1H-pyrrolo[2,3-b]pyridin-1-yl]acetic acid (100%). To a solution (0.09 M) of this material in DMF were added N-methylpiperazine (1.5 eq.), HATU (1.2 eq.), DIEA (3.0 eq.) and the resulting mixture was stirred at room temperature under nitrogen for 1.5 h. The solution was diluted with AcOEt and washed with saturated aqueous NH₄Cl solution, H₂O and brine then dried and concentrated to give methyl 3-cyclohexyl-1-[2-(4-methylpiperazin-1-yl)-2-oxoethyl]-2-phenyl-1H-pyrrolo[2,3-b]pyridine-5-carboxylate as a red oil. This material was dissolved in THF (0.18 M) and aqueous KOH (1 N, 3 eq.) was added The solution was stirred overnight at room temperature then adjusted to pH 3 by addition of aqueous HCl (1 N). The solution was diluted with MeCN/H₂O and purified by preparative HPLC (mobile phase: CH₃CN /H₂O containing 0.1% TFA) to afford the title compound (50%) as a solid.

¹H NMR (600 MHz, DMSO-d₆, 300 K) δ 1.19-1.33 (m, 3H), 1.66-1.68 (m, 1H), 1.77-1.82 (m, 6H), 2.59-2.61 (m, 1H), 2.79 (br s, 6H), 4.09-4.27 (m, 2H), 4.97-5.08 (m, 2H), 7.36-7.38 (m, 2H), 7.51-7.56 (m, 3H), 8.63 (d, J 1.7 Hz, 1H), 8.79 (d, J 1.7 Hz, 1H), 9.8 (br s, 1H); MS (ES⁺) m/z 461 (M+H)⁺.

### EXAMPLE 23

### 3-cyclohexyl-2-{3-[2-(dimethylamino)ethyl]phenyl}-1-[2-(dimethylamino)-2-oxoethyl]-1H-indole-6-carboxylic acid

Step 1: 1-tert-butyl 6-methyl 2-bromo-3-cyclohexyl-1H-indole-1,6-dicarboxylate

To a solution of methyl 2-bromo-3-cyclohexyl-1H-indole-6-carboxylate (0.1 M) in CH₂Cl₂, 4dimethylaminopyridine (1.05 eq.) and di-tert-butyl dicarbonate (1.05 eq.) were added. The mixture was stirred at room temperature for 1.5 h then diluted with CH₂CI₂ and washed with aqueous HCl (1 N) and brine. The organic phase was dried and concentrated to give the title compound (88 %) as a solid.

¹H NMR (300 MHz, DMSO-d₆, 300 K) δ 1.33-1.50 (m, 3H), 1.67 (s, 9H), 1.70-2.10 (m, 7H), 2.90-3.09 (m, 1H), 3.89 (s, 3H), 7.83 (d, J 8.2 Hz, 1H), 7.94 (d, J 8.2 Hz, 1H), 8.69 (s, 1H).

Step 2: 1-tert-butyl 6-methyl 3-cyclohexyl-2-(tributylstannyl)-1H-indole-1,6-dicarboxylate

To a solution (0.1 M) of 1-tert-butyl 6-methyl 2-bromo-3-cyclohexyl-1H-indole-1,6-dicarboxylate (from Step 1) in TBF, BuLi (1.3 eq, 1.6 N in hexane) was added dropwise at −78° C. After 15 min tributyl(chloro)stannane (1.2 eq.) was added dropwise and the mixture was allowed to warm to room temperature, then quenched with H₂O and EtOAc. The organic phase was separated then washed with brine, and dried. Removal of the solvent afforded a residue that was purified by flash chromatography (2% EtOAc in petroleum ether) to afford the title compound (65%) as solid.

¹H NMR (300 MHz, CDCl₃, 300 K) δ 0.88 (t, J 7.1 Hz, 9H), 0.94-1.19 (m, 6H), 1.20-1.46 (m, 9H) 1.48-1.62 (m, 9H), 1.68 (s, 9H), 1.70-2.12 (m, 4H), 2.78-2.95 (m, 1H), 3.94 (s, 3H), 7.76 (d, J 8.4 Hz, 1H), 7.83 (d, J 8.4 Hz, 1H), 8.74 (s, 1H).

Step 3: 1-tert-butyl 6-methyl 3-cyclohexy-2-{3-[2-(dimethylamino)ethyl]phenyl}-1H-indole-1.6-dicarboxylate

To a solution of 1-tert-butyl 6-methyl 3-cyclohexyl-2-(tributylstannyl)-1H-indole-1,6-dicarboxylate (from Step 2) in dioxane (0.05 M), CsF (8.8 eq) and [2-(3-bromophenyl)ethyl]dimethylamine (1.5 eq) were added. The resulting mixture was degassed with nitrogen and then Pd₂(dba)₃ (0.1 eq) and t-Bu₃P (0.4 eq) were added. The solution was refluxed for 5 h then cooled room temperature and diluted with EtOAc. The organic phase was separated then washed with brine and dried. Removal of the solvent afforded a residue that was purified by flash chromatography (3% MeOH in CH₂Cl₂ and 0.5% of triethylamine) to give the tide compound (68 %) as oil.

¹H NMR (300 MHz, DMSO-d₆, 300 K) δ 1.16 (s, 9H), 1.24-1.40 (m, 4H), 1.60-1.89 (m, 6H), 2.19 (s, 6H), 2.50 (m, 3H, under DMSO signal), 2.78 (t, J 7.1 Hz, 2H), 3.90 (s, 3H), 7.17 (d, J 7.3 Hz, 1H), 7.22 (s, 1H), 7.32 (d, J 7.3 Hz, 1H), 7.40 (t, J 7.3 Hz, 1H), 7.87 (d, J 8.4 Hz, 1H), 7.95 (d, J 8.4 Hz, 1H), 8.83 (s, 1H); MS (ES⁺) m/z 505 (M+H)³⁰ .

Step 4: 3-cyclohexyl-2-{3-[2-(dimethylamino)ethyl]phenyl{1-[2-(dimethylamino)-2-oxoethyl]-1H-indole-6-carboxylic acid

A solution (0.1 M) of 1-tert-butyl 6-methyl 3-cyclohexyl-2-{3-[2-(dimethylamino)ethyl]phenyl}-1H-indole-1,6-dicarboxylate (from Step 3) in a 1:1 mixture of TFA/CH₂Cl₂ was stirred at room temperature for 1 h. The solution was concentrated to afford a solid that was taken up in DMF. The resulting solution (0.1 M) was treated with NaH (2.5 eq) and stirred at room temperature for 0.5 h, then 2-chloro-N,N-dimethylacetamide (1.3 eq.) was added as DMF solution (0.1 M). After 3.5 h the mixture was quenched with aqueous HCl (1 N) and diluted with EtOAc. The organic phase was washed with brine, dried and concentrated. The crude compound was dissolved in CH₂CI₂ and the resulting mixture (0.05 M) was treated with BBr₃ (3 eq.). After 1 h the reaction was quenched with water and concentrated. The residue was purified by HPLC to give the title compound as solid (30%).

¹H NMR (300 MHz, DMSO-d₆, 300 K) δ 1.10-1.36 (m, 3H), 1.60-2.01 (m, 7H), 2.48-2.56 (m, 1H), 2.82 (s, 3H), 2.85 (s, 3H), 2.86 (s, 3H), 2.93 (s, 3H), 3.01-3.10 (m, 2H), 3.31 (m, 2H, partially obscured by residual H₂O signal), 4.88 (s, 2H), 7.19-7.27 (m, 2H), 7.42 (d, J 7.5 Hz, 1H), 7.52 (t, J 7.5 Hz, 1H), 7.66 (d, J 8.4 Hz, 1H), 7.84 (d, J 8.4 Hz, 1H), 7.95 (s, 1H); MS (ES⁺) m/z 476 (M+H)⁺.

### EXAMPLE 24

### 3-cyclohexyl-1-[2-(dimethylamino)prop-2-en-1-yl]-2-(2-methyl-1,2,3,4-tetrahydroisoquinolin-7-yl)-1H-indole-6-carboxylic acid

Step 1: N-[2-(4-bromohenyl)ethyl]-2,2,2-trifluoroacetamide

A solution of [2-(4-bromophenyl)ethyl]amine in trifluoroacetic anhydride was stirred for 1 h then quenched with H₂O. The white precipitate was filtered and dried to afford the title compound (97%).

¹H NMR (300 MHz, DMSO-d₆, 300 K) δ 2.80 (t, J 7.3 Hz, 2H), 3.33-3.50 (m, 2H), 7.19 (d, J 8.2 Hz, 2H), 7.50 (d, J 8.2 Hz, 2H), 9.40-9.53 (m, 1H).

Step 2: 7-bromo-2-(trifluoroacetyl)-1,2,3,4-tetrahydroisoquinoline

To a solution (0.3 M) of N-[2-(3-bromophenyl)ethyl]-2,2,2-trifluoroacetamide (from Step 1) in a 3:2 mixture of conc. sulfuric acid and acetic acid, paraformaldehyde (1.6 eq.) was added. The resulting solution was stirred overnight at room temperature, then diluted with EtOAc and washed with saturated aqueous NaHCO₃ solution and brine. The organic phases were concentrated and dried to give the title compound (83%) as an oil.

¹H NMR (300 MHz, DMSO-d₆, 300 K) δ 2.82-2.93 (m, 2H), 3.75-3.88 (m, 2H), 4.72-4.83 (m, 2H) 7.19 (d, J 8.2 Hz, 1H), 7.38-7.45 (m, 1H), 7.57 (d, J 8.2 Hz, 1H).

Step 3: 7-bromo-2-methyl-1,2,3,4-tetrahydroisoguinoline

To a solution (0.3 M) of 7-bromo-2-(trifluoroacetyl)-1,2,3,4-tetrahydroisoquinoline (from Step 2) in a 1:1 mixture of MeOH:H₂O, K₂CO₃ (3 eq.) was added. The mixture was stirred for 1 h, then diluted with EtOAc and washed with brine. The organic phase was dried and concentrated to give a residue that was dissolved in 1,2-dichloroethane. The resulting solution (0.1 M) was treated with formaldehyde (5 eq.) and Na(OAc)₃BH (5.2 eq.). The mixture was stirred overnight, then diluted EtOAc and washed with H₂O. The organic phase were dried, concentrated to give a residue that was purified by flash-chromatography (2:98 MeOH:CH₂Cl₂ containing 0.2% Et₃N) to give the title compound (60%) as oil.

¹H NMR (300 MHz, DMSO-d₆, 300 K) δ 2.32 (s, 1H), 2.53-2.62 (m, 2H), 2.70-2.82 (m, 2H), 3.40-3.51 (m, 2H), 7.07 (d, J7.7 Hz, 1H), 7.22-7.33 (m, 2H); MS (ES⁺) m/z 228 (M+H)⁺.

Step 4: 3-cyclohexyl-1-[2-(dimethylamino)prop-2-en-1-yl]-2-(2-methyl-1,2,3,4-tetrahydroisoluinolin-7-yl)-1H-indole-6-carboxylic acid.

Following the procedure described in Example 23, Steps 3 and 4, treatment of 1-tert-butyl 6-methyl 3-cyclohexyl-2-(tributylstannyl)-1H-indole-1,6-dicarboxylate (from Example 23, Step 2) with 7-bromo-2-methyl-1,2,3,4tetrahydroisoquinoline (from Example 23, Step 3) afforded a residue that was purified by RP-HPLC to afford the title compound (26%) as a solid.

¹H NMR (300 MHz, DMSO-d₆, 300 K) δ 1.10-1.42 (m, 3H), 1.60-1.80 (m, 5H), 1.81-2.05 (m, 2H), 2.50-2.55 (m, 1H, under DMSO), 2.82 (s, 3H), 2.85-3.10 (m, 2H), 2.95 (s, 6H), 3.10-3.30 (m, 2H), 4.25-4.65 (m, 2H), 4.89 (s, 2H), 7.15 (s, 1H), 7.24 (d, J 7.1 Hz, 1H), 7.42 (d, J 7.1 Hz, 1H), 7.66 (d, J 8.6 Hz, 1H), 7.84 (d, J 8.6 Hz, 1H), 7.95 (s, 1H); MS (ES⁺) m/z 474 (M+H)⁺.

### EXAMPLE 25

### 2-[3-cyclohexyl-6-(5-oxo-4,5-dihydro-1,2,4-oxadiazol-3-yl)-2-phenyl-1H-indol-1-yl]-N,N-dimethylacetamide

Step 1: 3-cyclohexyl-2-phenyl-1H-indole-6-carboxylic acid

To a solution (0.07 M) of methyl 3-cyclohexyl-2-phenyl-1H-indole-6-carboxylate in CH₂Cl₂ at 0° C. was added dropwise a solution (1.0 M) of BBr₃ (7.4 eq.) in CH₂Cl₂. The reaction mixture was stirred overnight then treated with a further 1.85 eq. of BBr₃ (1.0 M). After 4 h the reaction was diluted with EtOAc and the organic phase was washed with H₂O (2×) and brine then dried. Removal of the solvent in vacuo afforded the title compound (100%) as a solid.

¹H NMR (400 MHz, DMSO-d₆, 300 K) δ 1.10-1.50 (m, 4H), 1.60-1.95 (m, 6H), 2.50-2.55 (m, 1H, under DMSO), 7.31-7.34 (m, 2H), 7.49-7.55 (m, 3H) 7.65 (d, J 8.6 Hz, 1H), 7.83 (d, J 8.6 Hz, 1H), 8.0 (s 1H), 11.10 (s, 1H), 12.57 (br s, 1H); MS (ES⁺) m/z 320 (M+H)⁺.

Step 2: 3-cyclohexyl-2-phenyl-1H-indole-6-carbonitrile

To a solution (0.41 M) of 3-cyclohexyl-2-phenyl-1H-indole-6-carboxylic acid (from Step 1) in dry DMF were added dry pyridine (0.46 eq.), NH₄HCO₃ (1.26 eq.) and di-tert-butyl dicarbonate (1.3 eq.). The mixture was stirred overnight at room temperature then a further 1 eq. of di-tert-butyl dicarbonate were added and the mixture was stirred for a further 48 h. The mixture was diluted with ethyl acetate and H₂O and the organic layer was separated, washed with brine and dried. Removal of the solvent afforded a residue that was taken-up in a 1:2 mixture of dry CH₂Cl₂:CHCl₃ to give a solution (0.11 M). The solution was cooled to 0° C. then treated with Et₃N (3.0 eq) and (CF₃CO)₂O (1.3 eq). The mixture was stirred for 3 h at 0° C. then the volatiles were evaporated in vacuo and the residue was purified by flash chromatography on silica gel (15:85 EtOAc/petroleum ether) to afford the title compound (80%) as a solid.

¹H NMR (300 MHz, DMSO-d₆, 300 K) δ 1.20-1.28 (m, 4H), 1.45-1.85 (m, 4H), 1.90-2.08 (m, 2H), 2.75-2.81 (m, 1H), 7.27 (d, J 8.4 Hz, 1H), 7.50-7.58 (m, 5H), 7.74 (s, 1H), 7.95 (d, J 8.4 Hz, 1H), 11.67 (br s, 1H); MS (ES⁺) m/z 301 (M+H)⁺.

Step 3: tert-butyl (6-cyano-3-cyclohexyl-2-phenyl-1H-indol-1-yl)acetate

NaH (1.4 eq) was added to a solution (0.22 M) of 3-cyclohexyl-2-phenyl-1H-indole-6-carbonitrile (from Step 2) in DMF. The reaction mixture was stirred for 1 h at then treated with tert-butyl bromoacetate (2.0 eq) and warmed to 50° C. After 2 h the reaction mixture was diluted with EtOAc and aqueous HCl (1 N). The organic phase was washed with H₂O and brine then dried. Removal of the solvent in vacuo afforded the title compound (95%) as a solid.

1H NMR (400 MHz, DMSO-d₆, 300 K) δ 1.10-1.20 (m, 4H), 1.28 (s, 9H), 1.60-1.85 (m, 6H), 2.50-2.55 (m, 1H, under DMSO), 4.75 (s, 2H), 7.31-7.34 (m, 2H), 7.40 (d, J 8.9 Hz, 1H), 7.52-7.57 (m, 3H), 7.95 (d, J 8.9 Hz, 1H), 8.10 (s, 1H); MS (ES⁺) m/z 415 (M+H)⁺.

Step 4: tert-butyl [3-cyclohexyl-6-(5-oxo-4,5-dihydro-1,2,4-oxadiazol-3-yl)-2-phenyl-1H-indol-1-yl]acetate

ⁱPr₂NEt was added (10 eq.) to a solution (0.11 M) of tert-butyl (6-cyano-3-cyclohexyl-2-phenyl-1H-indol-1-yl)acetate (from Step 3) in MeOH. After 5 min. hydroxylamine hydrochloride (10 eq) was and the reaction mixture was stirred for 48 h. The mixture was diluted with EtOAc and the organic phase was washed with H₂O (2×) and brine, then dried. Removal of the solvent in vacuo afforded a residue that was taken up in dioxane. This solution (0.075 M) was treated with carbonyldiimidazole (1.2 eq.) then heated to 70° C. After 0.5 h the mixture was cooled and the volatiles were evaporated in vacuo. The residue was taken up in H₂O and extracted with EtOAc. The organic phase was washed with brine and dried, then the solvent was evaporated in vacuo to give a residue that was purified by flash chromatography on silica gel (3:7 EtOAc/petroleum ether and 0.1% of acetic acid) to afford the title compound (40%) as a white solid.

¹H NMR (300 MHz, DMSO-d₆, 300 K) δ 1.21-1.33 (m, 2H), 1.29 (s, 9H), 1.63-1.90 (m, 8H), 2.50-2.58 (m, 1H, under DMSO), 4.66 (s, 2H), 7.32-7.40 (m, 2H), 7.49-7.53 (m, 4H), 7.92-7.95 (m, 2H), 12.81 (br s, 1H); MS (ES⁺) m/z 474 (M+H)⁺.

Step 5: [3:cyclohexyl-6-(5-oxo-4,5-dihydro-1,2,4-oxadiazol-3-yl)-2-phenyl-1H-indol-1-yl]acetic acid

A solution (0.14 M) of tert-butyl [3-cyclohexyl-6-(5-oxo-4,5-dihydro-1,2,4-oxadiazol-3-yl)-2-phenyl-1H-indol-1-yl]acetate (from Step 4) in dry CH₂Cl₂ was treated with TFA (90 eq.). The mixture was stirred for 2 h then the solvent was evaporated in vacuo. The residue was then dried overnight under reduced pressure to afford the title compound (92%) as a solid.

¹H NMR (400 MHz, DMSO-d₆, 300 K) δ 1.21-1.43 (m, 3H), 1.52-1.90 (m, 7H), 2.50-2.58 (m, 1H, under DMSO), 4.55 (s, 2H), 7.33-7.38 (m, 2H), 7.50-7.60 (m, 4H), 7.91 (s, 1H), 7.95 (d, J 8.0 Hz, 1H), 12.82 (br s, 1H), 13.05 (br s, 1H); MS (ES⁺) m/z 418 (M+H)⁺.

Step 6: 2-[3-cyclohexyl-6-(5-oxo-4,5-dihydro-1,2,4-oxadiazol-3-yl)-2-phenyl-1H-indol-1-yl]-N,N-dimethylacetamide

To a solution (0.060 M) of [3-cyclohexyl-6-(5-oxo-4,5-dihydro-1,2,4-oxadiazol-3-yl)-2-phenyl-1H-indol-1-yl]acetic acid (from Step 5) in CH₂Cl₂ were added ⁱPr₂NEt (1.1 eq.) and dimethylamine (1.1 eq.). After 5 min., TBTU (1.1 eq) was added and the reaction mixture was then stirred overnight. The solvent was evaporated in vacuo and the residue was purified by RP-HPLC to afford the title compound (30%) as a white powder.

¹H NMR (400 MHz, DMSO-₆, 300 K) δ 1.20-1.43 (m, 3H), 1.63-1.80 (m, 5H), 1.83-1.90 (m, 2H), 2.50-2.58 (m, 1H, under DMSO), 2.85 (s, 3H), 2.95 (s, 3H), 4.56 (s, 2H), 7.31-7.36 (m, 2H), 7.47-7.56 (m, 4H), 7.79 (s, 1H), 7.93 (d, J 8.2 Hz, 8.2 Hz, 1H), 12.85 (br s, 1H); MS (ES⁺) m/z 445 (M+H)⁺.

### EXAMPLE 26

### 3-[3-cyclohexyl-1-{2-[4-(dimethylamino)piperidin-1-yl]-2-oxoethyl}-2-(3-furyl)-1H-indol-6-yl]-1,2,4-oxadiazol-5(4H)-one

Step 1: 2-bromo-3-cyclohexyl-1H-indole-6-carboxylic acid

KOH (3.0 eq) was added at room temperature to a solution (0.071 M) of methyl 2-bromo-3-cyclohexyl-1H-indole-6-carboxylate in a 1:1:2 mixture of TBF:MeOH:H₂O. The reaction mixture was stirred at 70° C. for 5 h then cooled and concentrated in vacuo. The residue was treated with aqueous HCl (1 N) and the precipitate was collected by filtration. After washing with H₂O and petroleum ether, the solid was dried under reduced pressure to afford the title compound (98%) as a white solid.

¹H NMR (300 MHz, DMSO-d₆, 300 K) δ 1.24-1.50 (m, 3H), 1.70-1.88 (m, 7H), 2.50-2.77 (m, 1H, under DMSO), 7.55 (d, J 8.4 Hz, 1H), 7.72 (d, J 8.4 Hz, 1H), 7.93 (s, 1H), 12.03 (s, 1H), 12.65 (br s, 1H); MS (ES⁺) m/z 322 (M+H)⁺.

Step 2: 2-bromo-3-cyclohexyl-1H-indole-6-carbonitrile

A solution (0.70 M) of 2-bromo-3-cyclohexyl-1H-indole-6-carboxylic acid (from Step 1) in dry DMF was treated with pyridine (0.26 eq.), NH₄HCO₃ (3.78 eq.) and di-tert-butyl dicarbonate (3.90 eq.). The reaction mixture was stirred overnight then diluted with EtOAc. The organic phase was washed with H₂O (twice), brine and dried, then the volatiles were removed to give a residue that was taken up in a 1:2 mixture of CH₂Cl₂:CHCl₃. The resulting solution (0.056 M) was treated with Et₃N (3.0 eq.) and (CF₃CO)₂O (1.3 eq.) at 0° C. After stirring for 3 h at 0° C. the solution was concentrated in vacuo and the residue was purified by flash chromatography on silica gel (15:85 EtOAc/petroleum ether) to afford the title compound (51%) as a solid.

¹H NMR (300 MHz, DMSO-d₆, 300 K) δ 1.24-1.50 (m, 3H), 1.75-1.90 (m, 7H), 2.50-2.80 (m, 1H, under DMSO), 7.24 (d, J 8.2 Hz, 1H), 7.70 (s, 1H), 7.88 (d, J 8.2 Hz, 1H), 12.24 (s, 1H); MS (ES⁺) m/z 303 (M+H)⁺.

Step 3: tert-butyl (2-bromo-6-cyano-3-cyclohexyl-1H-indol-1-yl)acetate

Following the procedure described in Example 25, Step 3, treatment of 2-bromo-3-cyclohexyl-1H-indole-6-carbonitrile (from Step 2) with NaH (1.4 eq) and tert-butyl bromoacetate (2.0 eq) afforded the title compound (88%) as a solid.

¹H NMR (400 MHz, DMSO-d₆, 300 K) δ 1.24-1.50 (m, 3H), 1.40 (s, 9H), 1.75-1.90 (m, 7H), 2.50-2.84 (m, 1H, under DMSO), 5.10 (s, 2H), 7.37 (d, J 9.0 Hz, 1H), 7.90 (d, J 9.0 Hz, 1H), 8.18 (s, 1H); MS (ES⁺) m/z 417 (M+H)⁺.

Step 4: tert-butyl [2-bromo-3-cyclohexyl-6-(5-oxo-4,5-dihydro-1,2,4-oxadiazol-3-yl)-1H-indol-1-yl]acetate

Following the procedure described in Example 25, Step 4, treatment of tert-butyl (2-bromo-6-cyano-3-cyclohexyl-1H-indol-1-yl)acetate (from Step 3) with ⁱPr₂NEt (10 eq.), hydroxylamine hydrochloride (10 eq.) and carbonyldiimidazole (1.2 eq.) afforded the title compound (38%) as a solid.

¹H NMR (400 MHz, DMSO-d₆, 300 K) δ 1.24-1.50 (m, 3H), 1.41 (s, 9H), 1.73-1.95 (m, 7H), 2.50-2.86 (m, 1H, under DMSO), 5.02 (s, 2H), 7.49 (d, J 8.0 Hz, 1H), 7.91 (d, J 8.0 Hz, 1H), 7.98 (s, 1H) 12.83 (br s, 1H); MS (ES⁺) mn/z 476 (M+H)⁺.

Step 5: [2-bromo-3-cyclohexyl-6-(5-oxo-4,5-dihydro-1,2,4-oxadiazol-3-yl -1H-indol-1-yl]acetic acid

Following the procedure described in Example 25, Step 5, treatment of tert-butyl [2-bromo-3-cyclohexyl-6-(5-oxo-4,5-dihydro-1,2,4-oxadiazol-3-yl)-1H-indol-1-yl]acetate (from Step 4) with TPA (90 eq. afforded the title compound (90%) as a solid.

¹H NMR (400 MHz, DMSO-d₆, 300 K) δ 1.24-1.50 (m, 3H), 1.70-1.95 (m, 7H), 2.50-2.86 (m, 1H, under DMSO), 5.04 (s, 2H), 7.49 (d, J 8.0 Hz, 1H), 7.91 (d, J 8.0 Hz, 1H), 7.97 (s, 1H) 12.83 (br s, 1H), 13.30 (br s, 1H); MS (ES⁺) m/z 420 (M+H)⁺.

Step 6: 1-{[2-bromo-3-cyclohexyl-6-(5-oxo-4,5-dihydro-1,2,4-oxadiazol-3-yl)-1H-indol-1-yl]acetyl}-N,N-dimethylpiperidin-4-aminium trifluoroacetate

Following the procedure described in Example 25, Step 6, treatment of [2-bromo-3-cyclohexyl-6-(5-oxo-4,5-dihydro-1,2,4-oxadiazol-3-yl)-1H-indol-1-yl]acetic acid (from Step 5) with ⁱPr₂NEt (3.1 eq.), 4-(dimethylamino)piperidinium bis(trifluoroacetate) (1.1 eq), and TBTU (1.1 eq.) gave the title compound (40%) as a white solid.

¹H NMR (400 MHz, DMSO-d₆, 300 K) δ 1.24-1.50 (m, 4H), 1.70-1.95 (m, 6H), 2.0-2.17 (m, 2H), 2.50-2.57 (m, 1H, under DMSO), 2.60 (s, 6H), 2.60-2.90 (m, 1H), 3.15-3.28 (m, 1H), 3.50-3.40 (m, 2H), 4.25 (d, J 8 Hz, 1H), 4.45 (d, J 8 Hz, 1H), 5.24 (d, J 17 Hz, 1H), 5.32 (d, J 17 Hz, 1H), 7.45 (d, J 7.7 Hz, 1H), 7.89-7.93 (m, 2H) 9.60 (br s, 1H), 12.90 (br s, 1H); MS (ES⁺) m/z 530.

Step 7: 1-{[3-cyclohexyl-2-(3-furyl)-6-(5-oxo-4,5-dihydro-1,2,4-oxadiazol-3-yl)-1H-indol-1yl]acetyl}-N,N-dimethylpiperidin-4-aminium trifluoroacetate

Pd(PPh₃)₂Cl₂ (0.2 eq) was added to a solution (0.037 M) of the product of Step 6 in dry dioxane. After 15 min., 3-furylboronic acid (3 eq.) and an aqueous solution of Na₂CO₃ (5.7 eq, 2 N) were added. The reaction mixture was heated under reflux for 1 h then cooled to room temperature. The volatiles were evaporated and the residue was purified by RP-HPLC to afford the title compound (23%) as a white solid.

¹H NMR (400 MHz, DMSO-d₆, 300 K) δ 1.24-1.50 (m, 4H), 1.70-1.95 (m, 6H), 2.0-2.17 (m, 2H), 2.50-2.59 (m, 3H), 2.60 (s, 6H), 3.05-3.10 (m, 1H), 3.33-3.40 (m, 1H), 3.45-3.48 (m, 1H), 4.09-4.13 (m, 1H), 4.41-4.44 (m, 1H), 5.05 (s, 2H), 6.52 (s, 1H), 7.45 (d, J 8.4 Hz, 1H), 7.82-7.84 (m, 2H), 7.88 (s, 1H), 7.93 (d, J 8.4 Hz, 1H), 9.60 (br s, 1H), 12.90 (br s, 1H); MS (ES⁺) m/z 518 (M+H)⁺.

### EXAMPLE 27

### 3-cyclohexyl-1-[2-(dimethylamino)-2-ozoethyl]-N-(ethylsulfonyl)-2-phenyl-1H-indole-6-carboxamide

A solution (0.05 M) of 3-cyclohexyl-1-[2-(dimethylamino)-2-oxoethyl]-2-phenyl-1H-indole-6-carboxylic acid (from Example 7) in CH₂Cl₂ was treated with DMAP (1.5 eq.) and ethane sulfonamide (1.5 eq.). EDCl (1.5 eq.) was added and the mixture was stirred overnight. The volatiles were evaporated under reduced pressure and the residue was purified by RP-HPLC to afford the tide compound (42%) as a solid.

¹H NMR (400 MHz, DMSO-d₆, 300 K) δ 1.20-1.43 (m, 3H), 1.28 (t, J 7.2 Hz, 3H) 1.63-1.80 (m, 7H), 2.50-2.58 (m, 1H, under DMSO), 2.80 (s, 3H), 2.90 (s, 3H), 3.5a (q, J 7.2 Hz, 2H), 4.57 (s, 2H), 7.31-7.36 (m, 2H), 7.49-7.56 (m, 3H), 7.66 (d, J 9.0 Hz, 1H), 7.85 (d, J 9.0 Hz, 1H), 8.05 (s, 1H), 10.76 (br s, 1H); MS (ES⁺) m/z 496 (M+H)⁺.

### EXAMPLE 28

### N-(benzylsulfonyl)-3-cyclohexyl-1-[2-(dimethylamino)-2-oxoethyl]-2-phenyl-1H-indole-6-carboxamide

A solution (0.05 M) of 3-cyclohexyl-1-[2-(dimethylamino)-2-oxoethyl]-2-phenyl-1H-indole-6-carboxylic acid (from Example 7) in CH₂Cl₂ was treated with a solution of oxalyl chloride (2.0 eq., 2 N in CH₂Cl₂). A catalytic quantity of DMF was added and the mixture was stirred for 1 h. The volatiles were evaporated at reduced pressure and the residue was taken-up in CH₂Cl₂. The resulting solution (0.05 M) was treated with DMAP (2.0 eq) and 1-phenylmethanesulfonamide (1.1 eq). The reaction mixture was stirred for 2 hours then the volatiles were evaporated under reduced pressure and the residue was triturated with a boiling mixture of H₂O: MeOH:acetone (1:2:2). The solid was separated by filtration, washed with CH₂Cl₂ and dried in vacuo to afford the title compound as a light yellow solid (40%).

¹H NMR (400 MHz, DMSO-d₆, 300 K) δ 1.15-1.38 (m, 3H), 1.63-1.91 (m, 7H), 2.50-2.58 (m, 1K, under DMSO), 2.78 (s, 3H), 2.89 (s, 3H), 4.77 (s, 2H), 4.92 (s, 2H), 7.31-7.39 (m, 7H), 7.49-7.56 (m, 3H), 7.66 (d, J 8.8 Hz, 1H), 7.86 (d, J 8.8 Hz, 1H), 8.10 (s, 1H), 11.71 (s, 1H); MS (ES⁺) m/z 558 (M+H)⁺.

### EXAMPLE 29

### 2-(4-chlorophenyl)-3-cyclohexyl-1-(2-morpholin-4-yl-2-ozoethyl)-1H-indole-6-carboxylic acid

Step 1: methyl 2-(4-chlorophenyl)-3-cyclohexyl-1H-indole-6-carboxylate

A solution (0.1 M) of methyl 2-bromo-3-cyclohexyl-1H-indole-6-carboxylate in DME and EtOH (5:2) was treated with 4chlorophenylboronic acid (1.2 eq.). Aqueous Na₂CO₃ (2 N, 8.5 eq.) was added and the solution was degassed, then treated with Pd(PPh₃)₄ (0.1 eq.). The mixture was heated at 80° C. for 4 h, then cooled and diluted with EtOAc and brine. The organic phase was separated and dried then concentrated under reduced pressure. The residue was purified by filtration through silica gel (1:9 EtOAc/petroleum ether) to afford the title compound (86%) as a solid.

¹H NMR (300 MHz, DMSO-d₆, 300 K) δ 1.22-1.50 (m, 3H), 1.68-1.89 (m, 5H), 1.90-2.11 (m, 2H), 2.77-2.95 (m, 1H), 3.88 (s, 3H), 7.56 (d, J 8.4 Hz, 2H), 7.62 (dd, J 8.4, 1.1 Hz, 1H), 7.64 (d, J 8.4 Hz, 2H), 7.87 (d, J 8.4 Hz, 1H), 8.02 (d, J 1.1 Hz, 1H), 11.57 (s, 1H).

Step 2: [2-(4chlorophenyl)-3-cyclohexyl-6-(methoxycarbonyl)-1H-indol-1-yl]acetic acid

A solution (0.1 M) of methyl 2-(4-chlorophenyl)-3-cyclohexyl-1H-indole-6-carboxylate (from Step 1) in DMF was treated portionwise with NaH (1.4 eq) at room temperature. The mixture was stirred for 1 h then treated dropwise with tert-butyl bromoacetate. After 2 h the reaction was diluted with EtOAc and aqueous HCl (1 N). The organic layer was separated, washed with aqueous HCl (1 N) and brine then dried. Removal of the solvent afforded a solid that was purified by flash chromatography (petroleum ether:EtOAc, 95:5) to afford a solid that was dissolved in a 1:1 mixture of CH₂Cl₂:TFA. The resulting solution (0.07 M) was stirred for 4 h then concentrated under reduced pressure. Trituration of the resulting oil with ether afforded the title compound (94%) as a solid.

¹H NMR (300 MHz, DMSO-d₆, 300 K) δ 1.15-1.42 (m, 3H), 1.61-196 (m, 7H), 2.54-2.65 (m, 1H), 3.89 (s, 3H), 4.78 (s, 2H), 7.38 (d, J 8.4 Hz, 2H), 7.64 (d, J 8.4 Hz, 2H), 7.71 (dd, J 8.4, 1.1 Hz, 1H), 7.89 (d, J 8.4 Hz, 1H), 8.07 (d, J 1.1 Hz, 1H), 12.99 (br s, 1H).

Step 3: 2-(4-chlorophenyl)-3-cyclohexyl-1-(2-morpholin-4-yl-2-oxoethyl)-1H-indole-6-carboxylic acid

A solution (0.04 M) of [2-(4-chlorophenyl)-3-cyclohexyl-6-(methoxycarbonyl)-1H-indol-1-yl]acetic acid (from Step 2) in CH₂Cl₂ was added to a glass tube containing PS-carbodiimide (3 eq). A solution (0.09 M) of morpholine (2.4 eq) in CH₂Cl₂ was added and the resulting mixture was combined to homogeneity using vortex shaking, then agitated for 48 h. PS-NCO resin (6.0 eq) was added along with further CH₂Cl₂ to ensure homogeneity. The mixture was agitated for 15 h then filtered. The filtrate was treated with a freshly prepared solution (1.52 M) of BBr₃ (4.0 eq) then stirred for 1 h. The solvent was removed and the residue treated with aqueous HCl (1 N). The resulting residue was dissolved in DMSO and purified by automated RP-HPLC to afford the title compound (9%) as a solid.

¹H NMR (400 MHz, DMSO-δ₆, 300 K) δ 1.13-1.42 (m, 3H), 1.61-1.99 (m, 7H), 2.54-2.66 (m, 1H), 3.38-3.47 (m, 4H), 3.47-3.60 (m, 4H), 4.94 (s, 2H), 7.35 (d, J 8.4 Hz, 2H), 7.63 (d, J 8.4 Hz, 2H), 7.67 (d, J 8.4 Hz, 1H), 7.84 (d, J 8.4 Hz, 1H), 7.99 (s, 1H), 12.57 (br s, 1H); MS (ES⁺) m/z 481 (M+H)⁺.

### EXAMPLE 30

### 3-cyclohexyl-2-(4-methoxyphenyl)-1-(2-morpholin-4-yl-2-oxoethyl)-1H-indole-6-carboxylic acid

Step 1: methyl 1-(2-tert-butoxy-2-oxoethyl)-3-cyclohexyl-2-(4-methoxyphenyl)-1H-indole-6-carboxylate

A solution (0.1 M) of methyl 2-bromo-1-(2-tert-butoxy-2-oxoethyl)-3-cyclohexyl-1H-indole-6-carboxylate (prepared as described in Example 1, Step 4) in DME and EtOH (5:2) was treated with 4methoxyphenylboronic acid (1.5 eq.). Aqueous Na₂CO₃ (8.5 eq., 2 N) was added and the solution was degassed, then treated with Pd(PPh₃)₄ (0.1 eq.). The mixture was heated at 80° C. for 2 h, then cooled and diluted with EtOAc and brine. The organic phase was separated, dried and concentrated under reduced pressure. The residue was purified by filtration through silica gel (5:95 EtOAc/petroleum ether) to give the title compound (81%) as a solid.

¹H NMR (300 MHz, DMSO-d₆, 300 K) δ 1.13-1.38 (m, 3H), 1.33 (s, 9H), 1.61-1.96 (m, 7H), 2.55-2.67 (m, 1H), 3.86 (s, 3H), 3.88 (s, 3H), 4.73 (s, 2), 7.11 (d, J 8.6 Hz, 2H), 7.27 (d, J 8.6 Hz, 2H), 7.69 (dd, J 8.4, 1.1 Hz, 1H), 7.86 (d, J 8.4 Hz, 1H), 7.86 (d, J 8.4 Hz, 1H), 8.06 (d, J 1.1 Hz).

Step 2: [3-cyclohexyl-6-(methoxycarbonyl)-2-(4-methoxyphenyl-1H-indol-1-yl]acetic acid

A solution (0.4 M) of methyl 1-(2-tert-butoxy-2-oxoethyl)-3-cyclohexyl-2-(4-methoxyphenyl)-1H-indole-6-carboxylate (from Step 1) in a 1:1 mixture of CH₂CI₂/TFA was stirred at 25° C. for 4 h. The mixture was concentrated and the residue was triturated with Et₂O to afford the tide compound (95%) as a solid.

¹H NMR (300 MHz, DMSO-d₆, 300 K) δ 1.13-1.36 (m, 3H), 1.62-1.95 (m, 7H), 2.56-2.67 (m, 1H), 3.86 (s, 3H), 3.89 (s, 3H), 4.74 (s, 2H), 7.12 (d, J 8.6 Hz, 2H), 7.28 (d, J 8.6 Hz, 2H), 7.69 (dd, J 8.5, 1.2 Hz, 1H), 7.86 (d, J 8.5 Hz), 8.02 (d, J 1.2 Hz, 1H), 12.98 (br s, 1H).

Step 3: 3-cyclohexyl-2-(4-methoxyphenyl)-1-(2-morpholin-4-yl-2-oxoethyl)-1H-indole-6-carboxylic acid

A solution (0.04 M) of [3-cyclohexyl-6-(methoxycarbonyl)-2-(4-methoxyphenyl)-1H-indol-1-yl]acetic acid in (from Step 2) CH₂Cl₂ was treated with DIPEA (4 eq.), HATU (2 eq.) and morpholine (1.5 eq.). The mixture was stirred at 25° C. for 12 h and then diluted with EtOAc and washed sequentially with aqueous HCl (1 N), NaOH (2 N) and brine. The dried organic layer was concentrated and the residue was dissolved in a 1:1 mixture of THF/H₂O. The resulting solution (0.05 M) was treated with aqueous KOH (4 eq., 1 N) then stirred at 70° C. for 16 h. The mixture was concentrated under reduced pressure and the residue was treated with aqueous HCl (1 N). After extraction with EtOAc the combined organic layer was washed with brine, dried, and concentrated to afford the title compound (91%) as a solid.

¹H NMR (300 MHz, DMSO-d₆, 300 K) δ 1.12-1.40 (m, 3n), 1.61-1.99 (m, 7H), 2.55-2.68 (m, 1H), 3.39-3.46 (m, 4H), 3.47-3.62 (m, 4H), 3.86 (s, 3H), 4.89 (s, 2H), 7.11 (d, J 8.6 Hz, 2H), 7.25 (d, J 8.6 Hz, 2H), 7.66 (d, J 8.4 Hz, 1H), 7.81 (d, J 8.4 Hz, 1H), 7.95 (s, 1H), 12.59 (br s, 1H); MS (ES⁺) m/z 477 (M+H)⁺.

### EXAMPLE 31

### 1-{[5-carboxy-3-cyclohexyl-2-(4-methoxyphenyl)-1H-indol-1-yl]acetyl}-N,N-dimethylpiperidin-4-aminium trifluoroacetate

Step 1: methyl 3-cyclohexyl-2-(trimethylsilyl)-1H-indole-5-carboxylate

Following the procedure described in Example 21, Step 2, reaction of methyl 4-amino-3-iodobenzoate with (cyclohexylethynyl)(trimethyl)silane (obtained as described in Example 21, Step 1) afforded the title compound (67%) as a solid.

¹H NMR (300 MHz, DMSO-d₆, 300 K) δ 0.38 (s, 9H), 1.23-1.52 (m, 3H), 1.61-2.03 (m, 7H), 2.77-2.95 (m, 1H), 3.86 (s, 3H), 7.45 (d, J 8.6 Hz, 1i), 7.68 (d, J 8.6 Hz, 1H), 8.37 (s, 1H), 10.78 (s, 1H).

Step 2: methyl 2-bromo-3-cyclohexyl-1H-indole-5-carboxylate

A solution (0.09 M) of methyl 3-cyclohexyl-2-(trimethylsilyl)-1H-indole-5-carboxylate (from Step 1) in CCl₄ was treated portionwise with N-bromosuccinimide (1.2 eq.). The mixture was stirred at room temperature for 1 h then diluted with a saturated aqueous solution of Na₂S₂O₃. After 3 h the organic phase was separated, washed with brine and dried. Removal of the solvent afforded a residue which was purified by flash chromatography on silica gel (EtOAc/petroleum ether, 5:95) to afford the title compound (94%) as a yellow solid.

¹H NMR (300 MHz, DMSO-d₆, 300 K) δ 1.28-1.53 (m, 3H), 1.64-1.98 (m, 7H), 2.76-2.92 (m, 1n), 3.86 (s, 3H), 7.37 (d, J 8.5 Hz, 1i), 7.72 (d, J 8.5 Hz, 1H), 8.31 (s, 1H), 12.04 (s, 1H).

Step 3: methyl 2-bromo-3-cyclohexyl-1-{2-[4-(dimethylamino)piperidin-1-yl]-2-oxoethyl}-1H-indole-5-carboxylate

Methyl 2-bromo-3-cyclohexyl-1H-indole-5-carboxylate (from Step 2) was alkylated and deprotected as described in Example 1, Steps 4 and 5 to afford [2-bromo-3-cyclohexyl-5-(methoxycarbonyl)-1H-indol-1-yl]acetic acid. A solution (0.3 M) of this material in CH₂Cl₂ was treated with N,N-dimethylpiperidinamine (1.5 eq.), DIPEA (4 eq.) and HATU (1.5 eq.). The solution was stirred at 20° C. for 12 h. The mixture was diluted with CH₂Cl₂ then washed with aqueous NaOH (2 N) and brine. The organic layer was dried and concentrated to give a residue that was purified by flash chromatography on silica gel (5:95 EtOAc/petroleum ether) to afford the title compound (70%) as a solid.

¹H NMR (300 MHz, DMSO-d₆, 300 K) δ 1.31-1.56 (m, 5H), 1.67-1.96 (m, 9H), 2.21 (s, 6H), 2.30-2.44 (m, 1H), 2.60-2.74 (m, 1H), 2.80-2.97 (m, 1H), 3.07-3.24 (m, 1H), 3.88 (s, 3H), 3.99-4.13 (m, 1H), 4.17-4.32 (m, 1H), 5.13-5.34 (m, 2H), 7.54 (d, J 8.6 Hz, 1H), 7.75 (d, J 8.6 Hz, 1H), 8.34 (s, 1H); MS (ES⁺) m/z 504, 506 (M+H)⁺.

Step 4: 1-{[5-carboxy-3-cyclohexyl-2-(4-methoxyphenyl)-1H-indol-1-yl]acetyl}-N,N-dimethylpiperidin-4-aminium trifluoroacetate

Following the procedure described in Example 1, step 7, treatment of methyl 2-bromo-3-cyclohexyl-1-{2-[4-(dimethylamino)piperidin-1-yl]-2-oxoethyl)-1H-indole-5-carboxylate (from Step 3) with 4-methoxyphenylboronic acid gave a residue that was purified by RP-HPLC to afford the title compound (40%) as a solid.

¹H NMR (400 MHz, DMSO-d₆, 300 K) δ 1.13-1.42 (m, 5H), 1.63-1.90 (m, 7H), 1.91-2.05 (m, 2H), 2.56-2.66 (m, 3H), 2.74 (s, 6H), 2.91-3.07 (m, 1H), 3.84 (s, 3H), 3.90-4.02 (m, 1H), 4.35-4.47 (m, 1H), 4.79-4.94 (m, 2H), 7.11 (d, J 8.2 Hz, 2H), 8.25 (d, J 8.2 Hz, 2H), 7.37 (d, J 8.6 Hz, 1H), 7.74 (d, J 8.6 Hz, 1H), 8.40 (s, 1H), 9.57 (br s, 1H), 12.50 (br s, 1H); MS (ES⁺) m/z 518 (M+H)⁺.

### EXAMPLE 32

### 1-{[5-carboxy-3-cyclohexyl-2-(3-furyl)-1H-indol-1-yl]acetyl}-N,N-dimethylpiperidin-4-aminium trifluoroacetate

Following the same procedure described in Example 31, Step 4, methyl 2-bromo-3-cyclohexyl-1-{2-[4-(dimethylamino)piperidin-1-yl]-2-oxoethyl}-1H-indole-5-carboxylate (from Example 31, Step 3) was treated with 3-furanboronic acid to afford the title compound (45%) as a solid.

¹H NMR (400 M&z, DMSO-d₆, 300 K) δ 1.22-1.52 (m, 5H), 1.69-1.94 (m, 7H), 1.96-2.08 (m, 2H), 2.56-2.66 (m, 1H), 2.68-2.86 (m, 7H), 3.00-3.13 (m, 1H), 3.98-4.16 (m, 1H), 4.34-4.51 (m, 1H), 5.00 (s, 2H), 6.52 (s, 1H), 7.41 (d, J 8.6 Hz, 1H), 7.74 (d, J 8.6 Hz, 1H), 7.81 (s, 1H), 7.89 (s, 1H), 8.39 (s, 1H), 9.60 (br s, 1H), 12.53 (br s, 1H); MS (ES⁺) m/z 479 (M+H)⁺.

### EXAMPLE 33

### (4-{[6-carboxy-2-(4-chlorophenyl)-3-cyclohexyl-1H-indol-1-yl]acetyl}morpholin-2-yl)-N,N-dinethylmethanaminium trifluoroacetate

Following the procedure described in Example 29, Step 3, treatment of [2-(4-chlorophenyl)-3-cyclohexyl-6-(methoxycarbonyl)-1H-indol-1-yl]acetic acid (from Example 29, Step 2) with dimethyl(morpholin-2-ylmethyl)amine afforded the title compound (9%) as a solid.

¹H NMR (400 MHz, DMSO-d₆, 340 K) δ 1.15-1.41 (m, 3H), 1.58-1.96 (m, 7H), 2.60 (m, 1H), 2.83 (s, 6H), 3.10-3.31 (m, 2H), 3.39-3.53 (m, 1H), 3.67-4.23 (br m, 6H), 4.76-5.11 (m, 2H), 7.36 (d, J 8.2 Hz, 2H), 7.60 (d, J 8.2 Hz, 2H), 7.68 (d, J 8.3 Hz, 1H), 7.83 (d, J 8.3 Hz, 1H), 7.97 (s, 1H), 9.44 (brs, 1H); MS (ES⁺) m/z 538 (M+H)⁺.

TABLE 1
Additional Examples (C-6 carboxylic acids)

| | Molecular |
|---|---|
| | Ion |
| STRUCTUTRE | [M + H]⁺ |
| 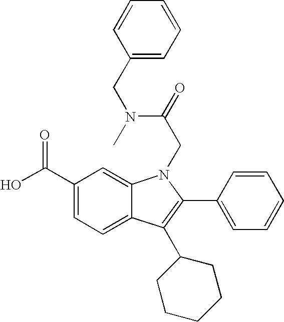 | 481 |
| 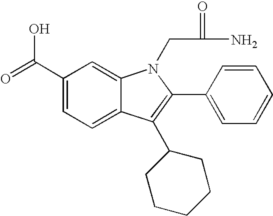 | 377 |
| 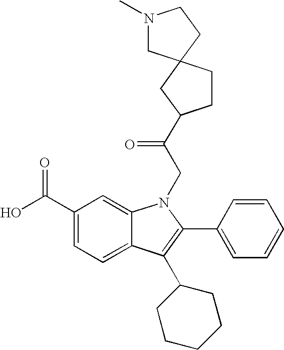 | 500 |
| 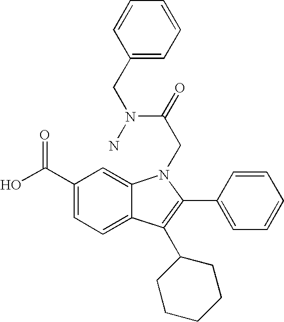 | 467 |
| 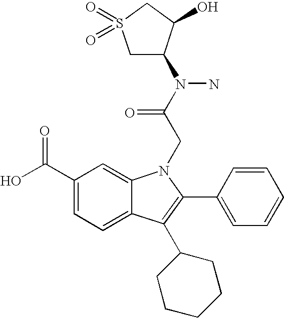 | 511 |
| 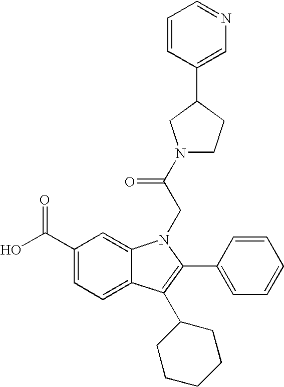 | 508 |
| 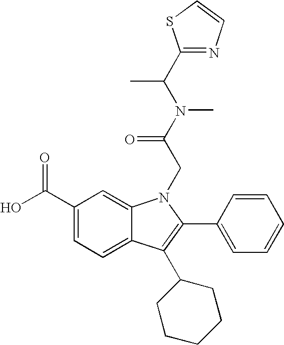 | 502 |
| 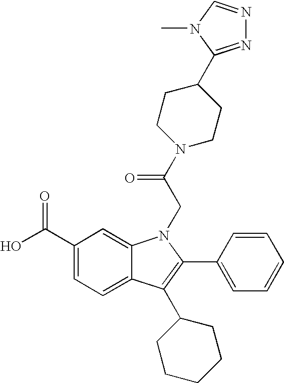 | 526 |
| 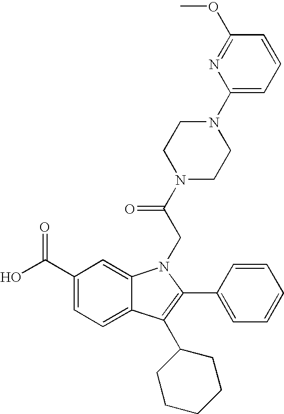 | 553 |
| 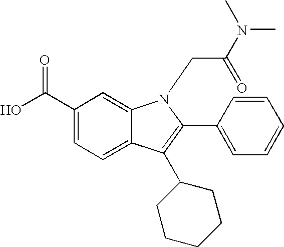 | 406 |
| 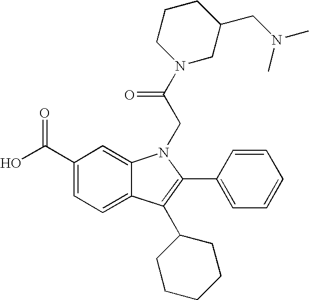 | 502 |
| 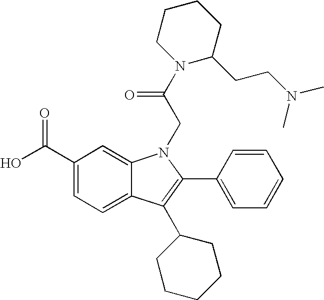 | 516 |
| 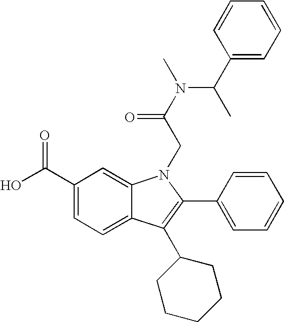 | 496 |
| 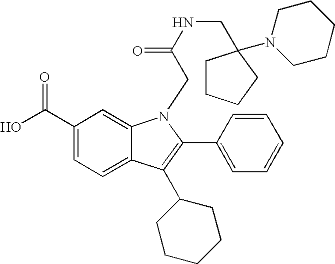 | 542 |
| 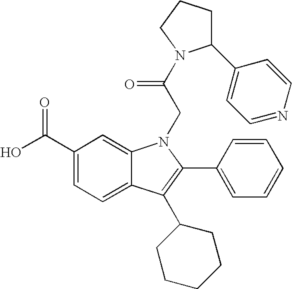 | 508 |
| 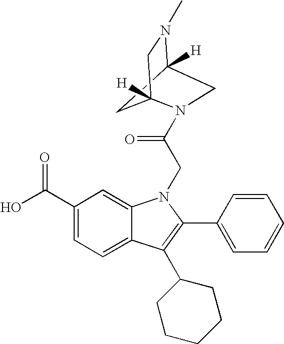 | 472 |
| 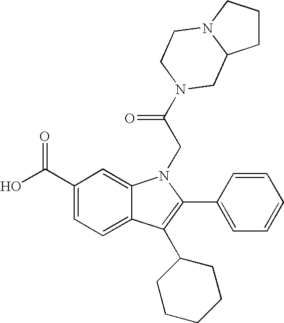 | 486 |
| 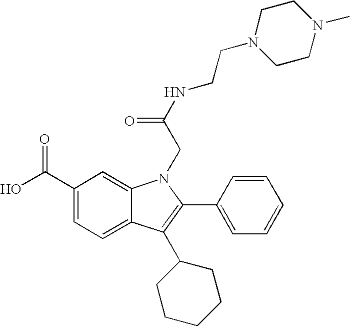 | 503 |
| 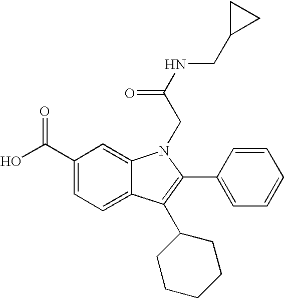 | 431 |
| 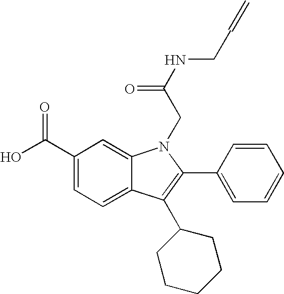 | 415 |
| 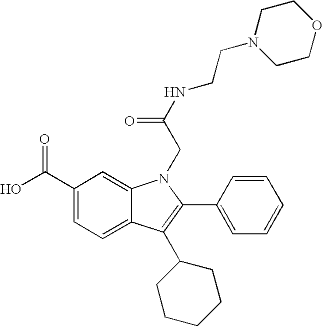 | 490 |
| 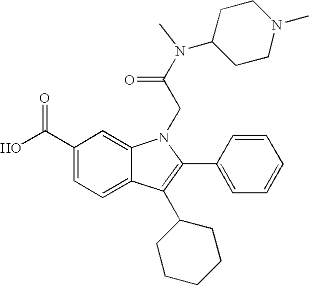 | 486 |
| 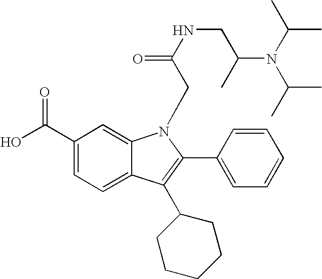 | 504 |
| 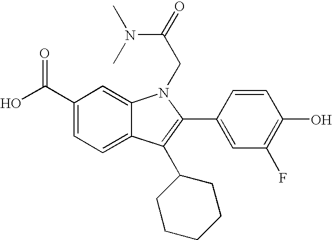 | 439 |
| 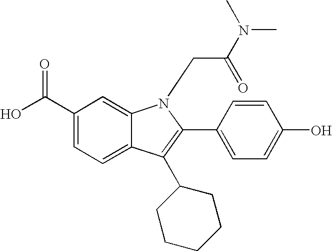 | 421 |
| 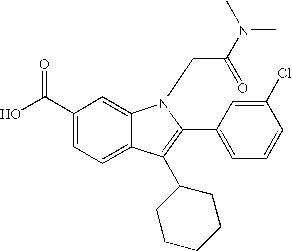 | 439 |
| 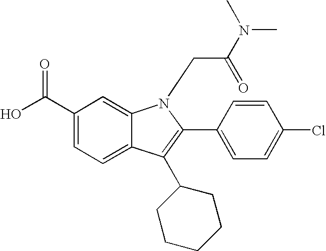 | 439 |
| 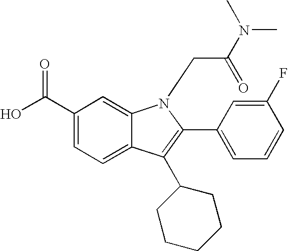 | 423 |
| 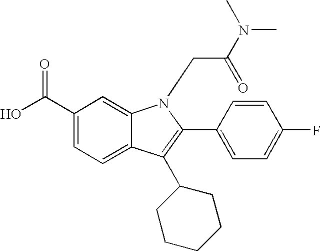 | 423 |
| 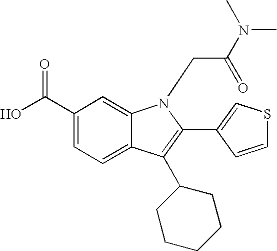 | 411 |
| 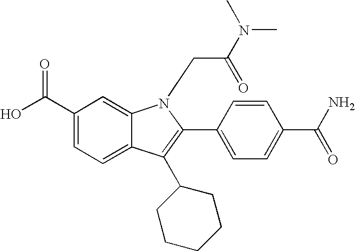 | 448 |
| 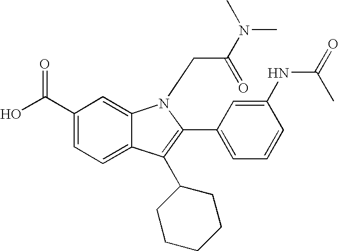 | 462 |
| 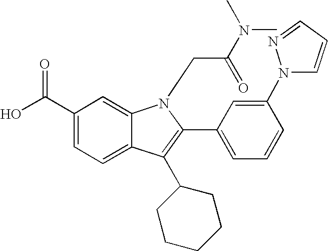 | 471 |
| 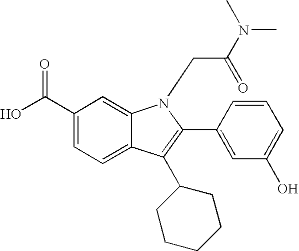 | 421 |
| 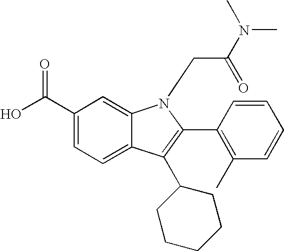 | 419 |
| 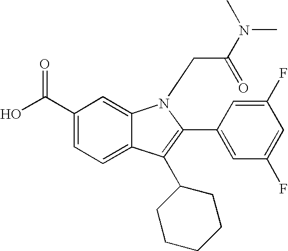 | 441 |
| 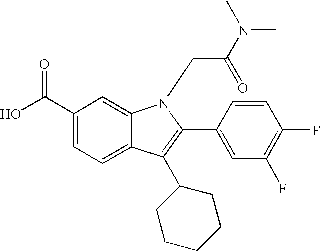 | 441 |
| 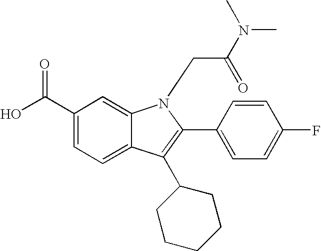 | 441 |
| 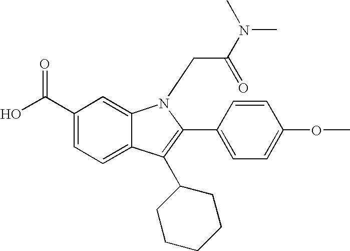 | 435 |
| 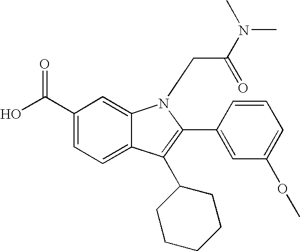 | 435 |
| 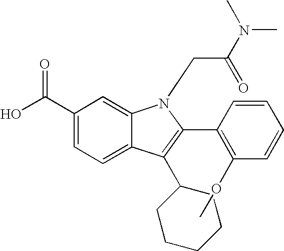 | 435 |
| 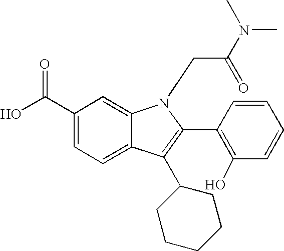 | 421 |
| 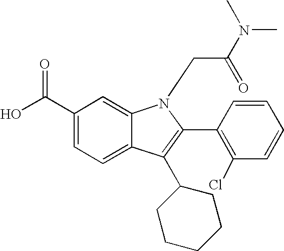 | 439 |
| 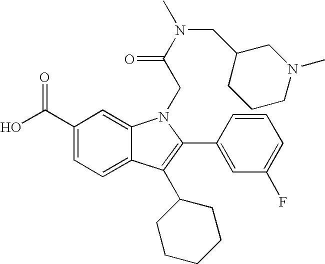 | 520 |
| 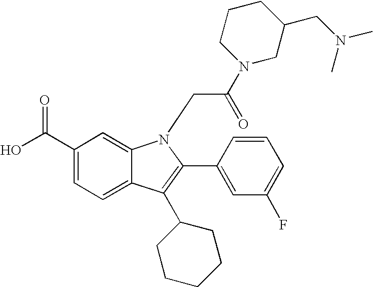 | 520 |

TABLE 2
Additional Examples

| | Molecular Ion |
|---|---|
| STRUCTURE | [M + H]⁺ |
| 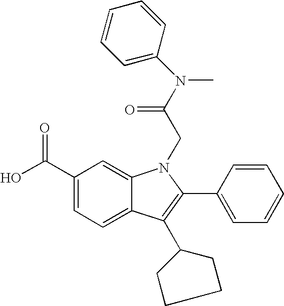 | 453 |
| 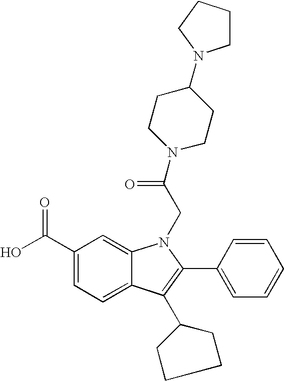 | 500 |
| 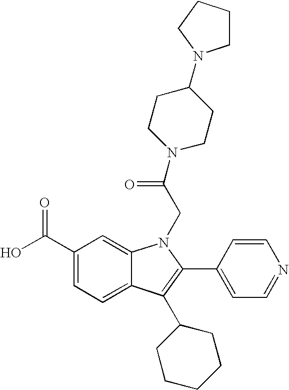 | 515 |
| 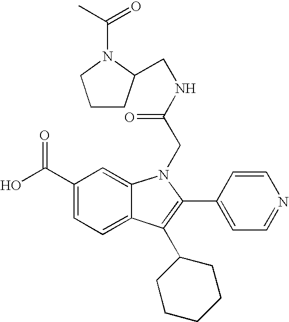 | 503 |
| 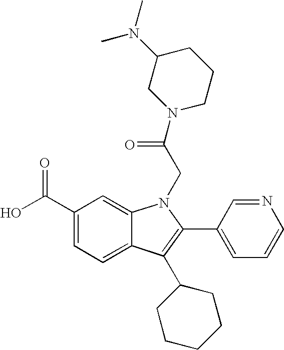 | 489 |
| 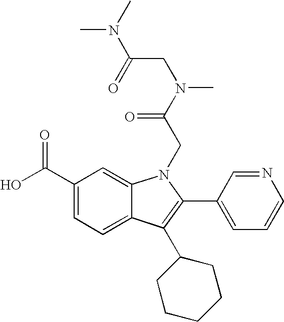 | 477 |
| 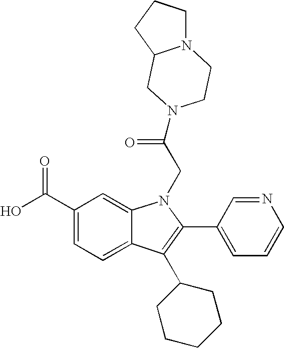 | 487 |
| 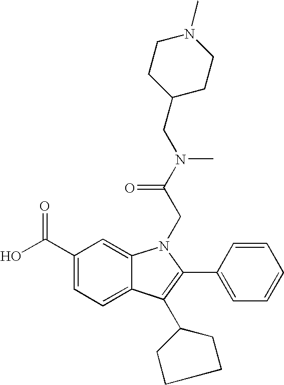 | 488 |
| 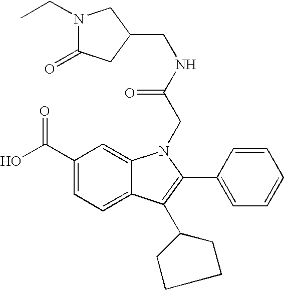 | 488 |
| 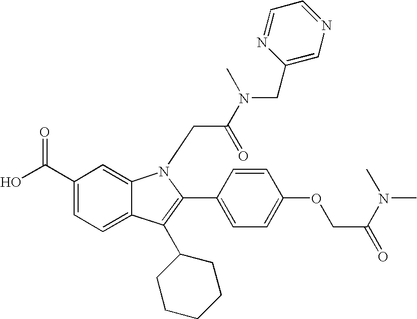 | 584 |
| 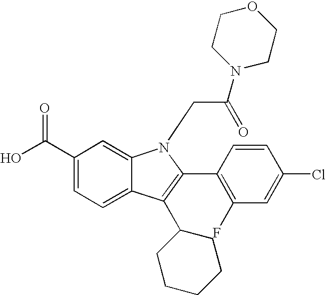 | 499 |
| 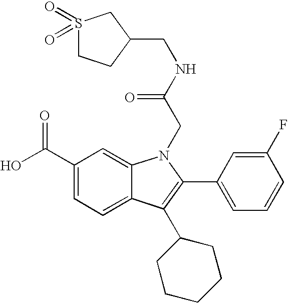 | 527 |
| 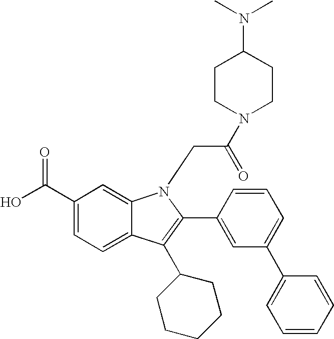 | 564 |
| 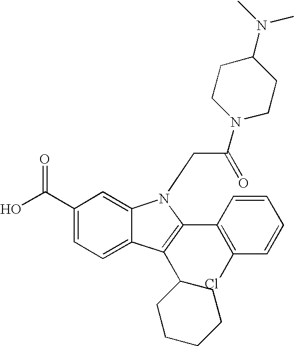 | 522 |
| 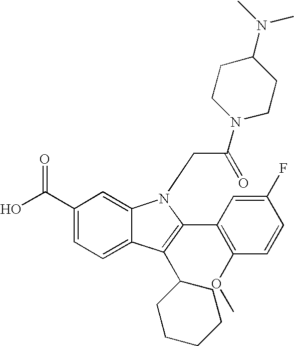 | 536 |
| 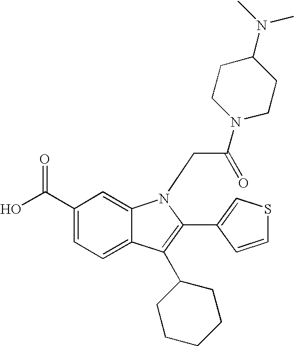 | 494 |
| 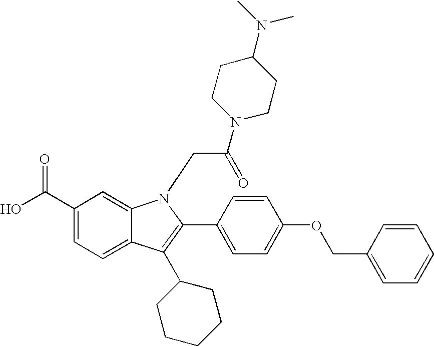 | 594 |
| 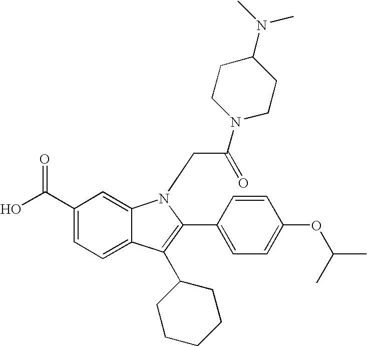 | 546 |
| 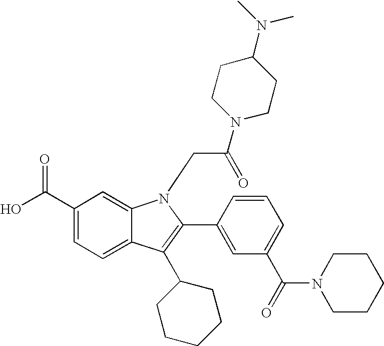 | 599 |
| 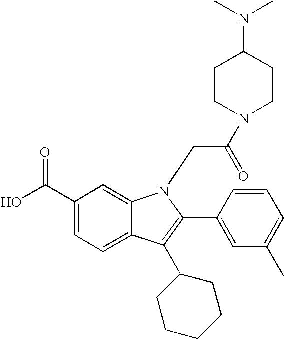 | 502 |
| 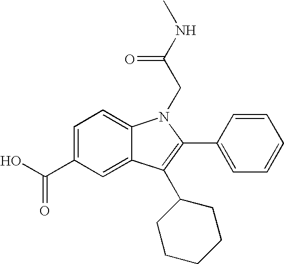 | 391 |
| 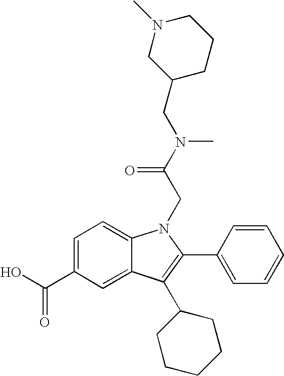 | 502 |
| 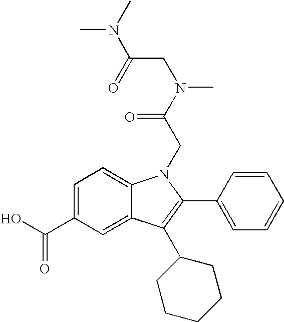 | 476 |
| 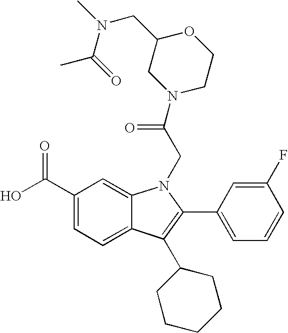 | 550 |
| 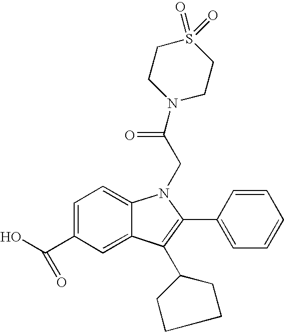 | 481 |
| 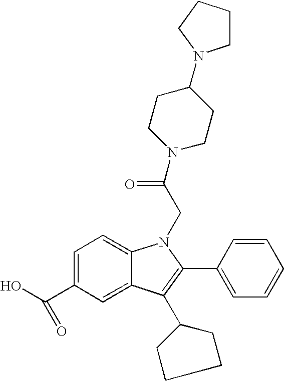 | 500 |
| 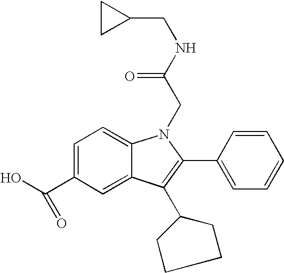 | 417 |
| 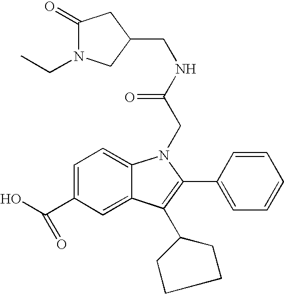 | 488 |
| 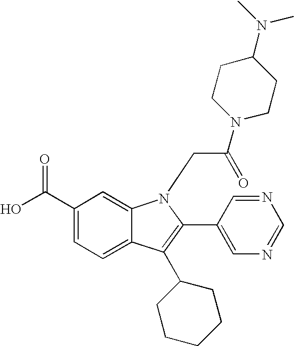 | 490 |
| 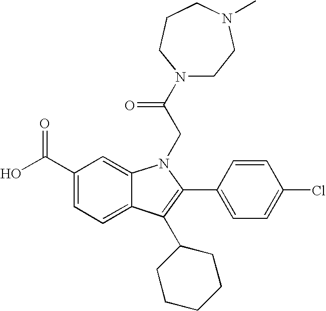 | 508 |
| 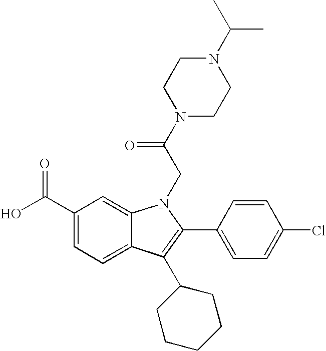 | 522 |
| 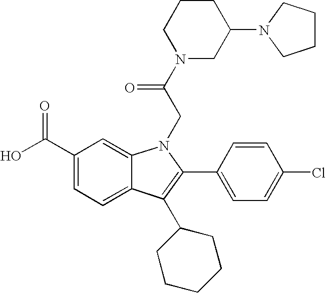 | 548 |
| 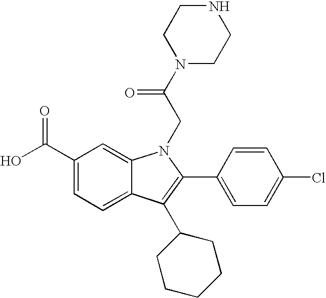 | 480 |
| 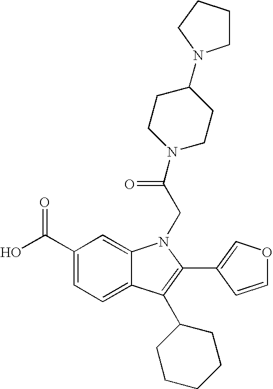 | 504 |
| 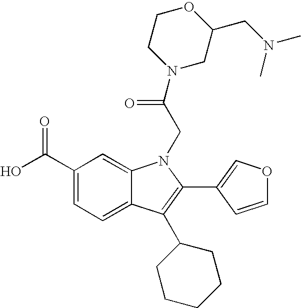 | 494 |
| 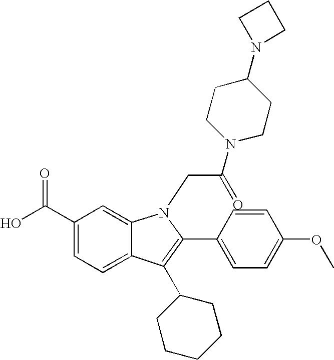 | 530 |
| 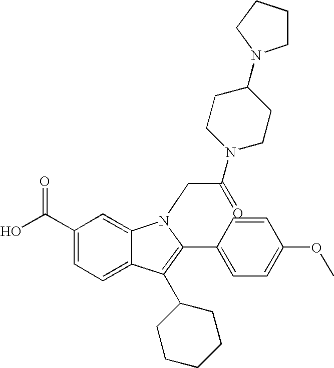 | 544 |
| 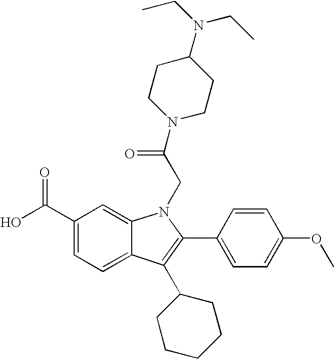 | 546 |
| 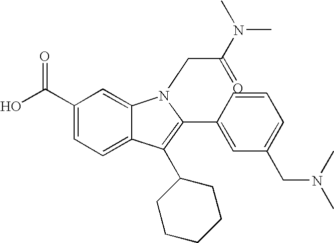 | 462 |
| 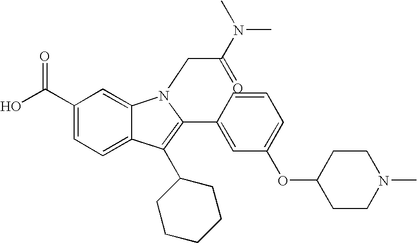 | 518 |
| 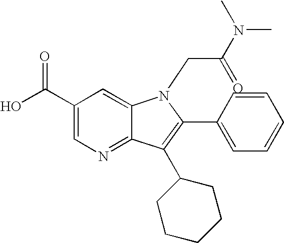 | 406 |
| 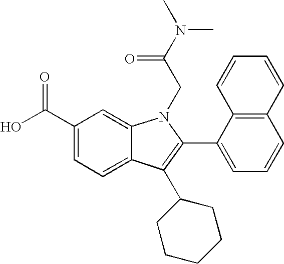 | 455 |
| 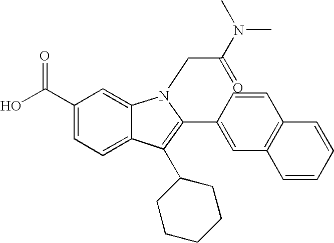 | 455 |
| 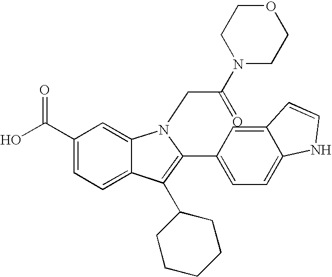 | 486 |
| 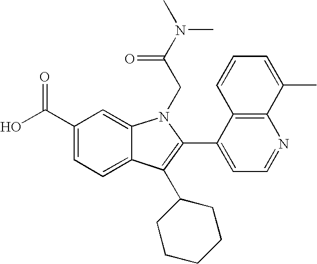 | 470 |

TABLE 3
C-6 Carboxamides

| | Molecular Ion |
|---|---|
| STRUCTURE | [M + H]⁺ |
| 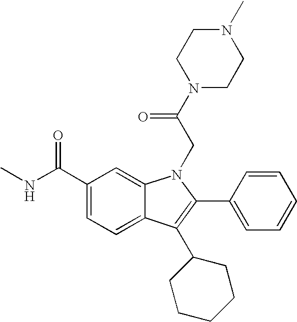 | 473 |
| 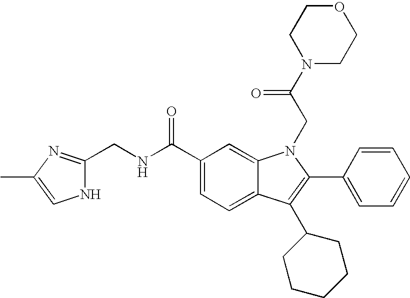 | 540 |
| 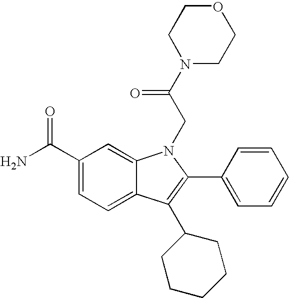 | 446 |
| 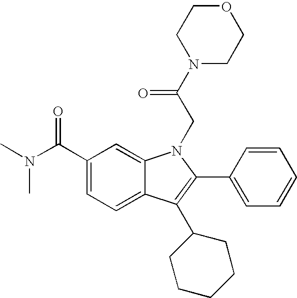 | 474 |
| 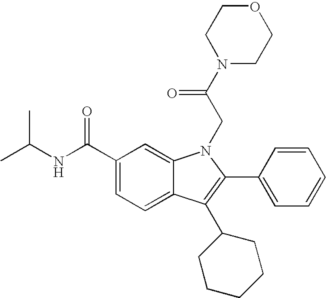 | 488 |
| 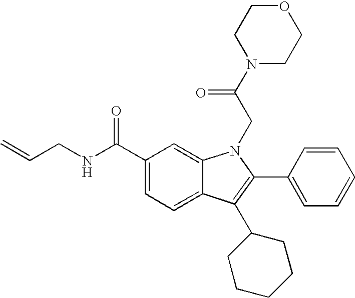 | 486 |
| 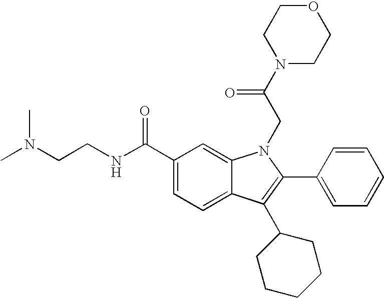 | 517 |
| 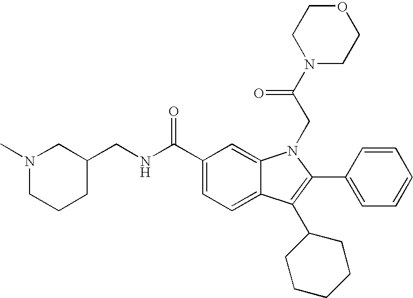 | 557 |
| 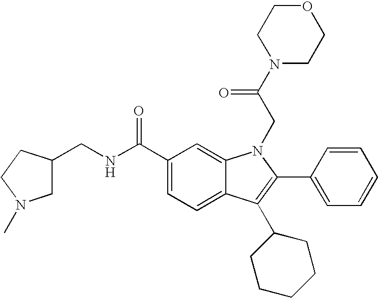 | 543 |
| 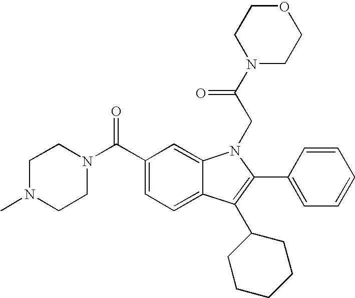 | 529 |
| 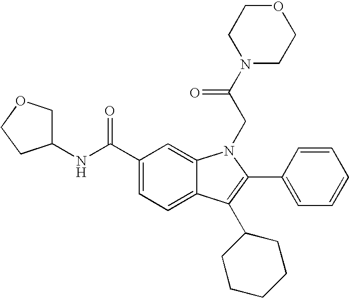 | 516 |
| 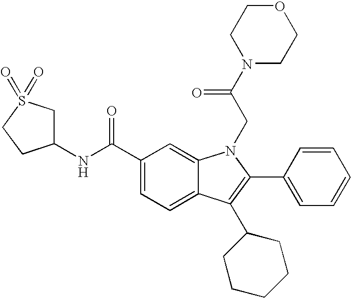 | 564 |
| 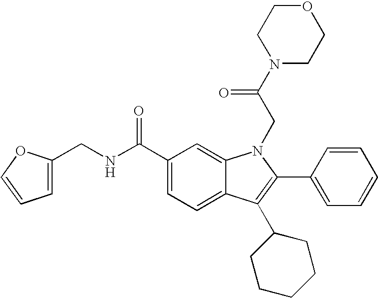 | 526 |
| 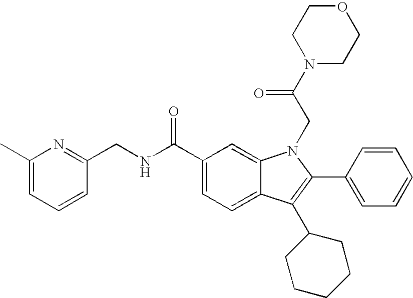 | 551 |
| 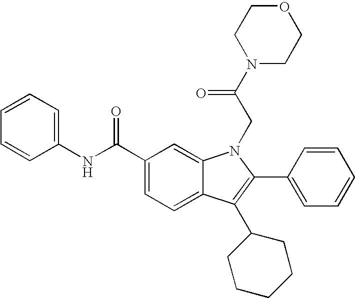 | 522 |
| 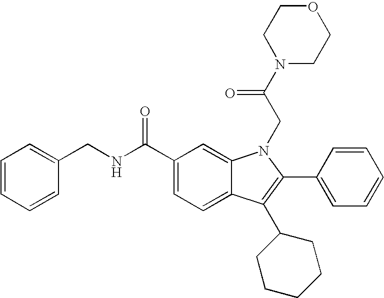 | 536 |
| 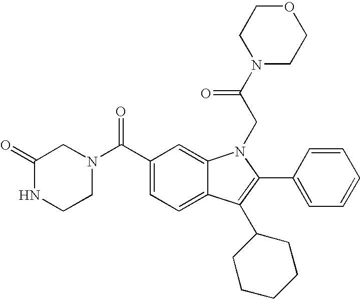 | 529 |
| 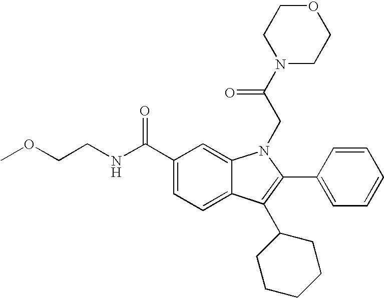 | 504 |
| 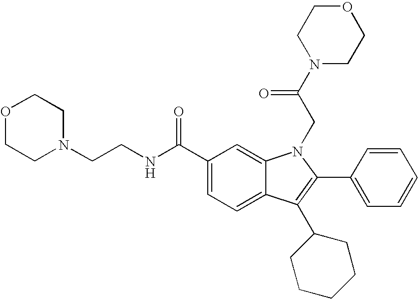 | 559 |
| 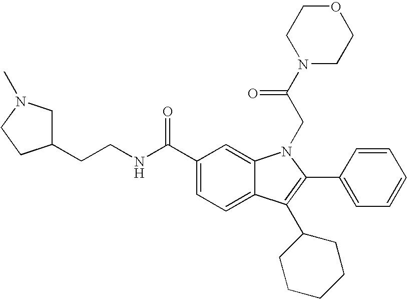 | 557 |
| 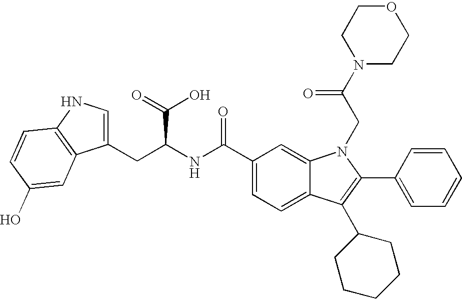 | 649 |
| 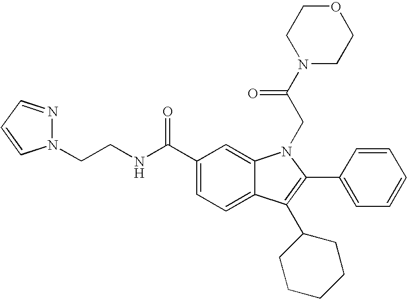 | 540 |

TABLE 4
C-6 Acid Replacements

| | Molecular Ion |
|---|---|
| STRUCTURE | [M + H]⁺ |
| 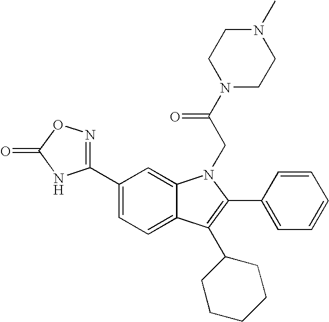 | 500 |
| 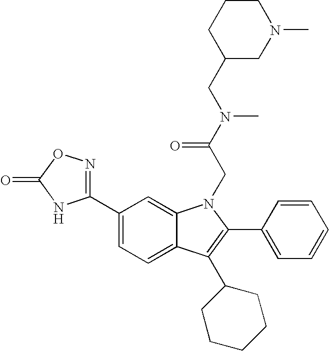 | 542 |
| 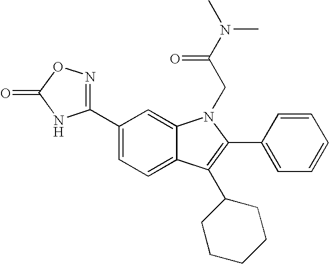 | 445 |
| 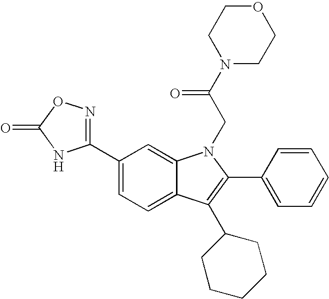 | 487 |
| 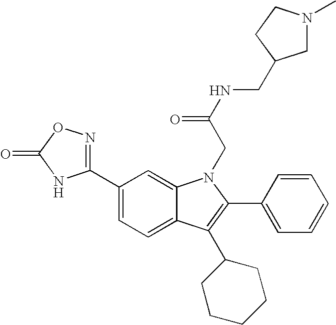 | 514 |
| 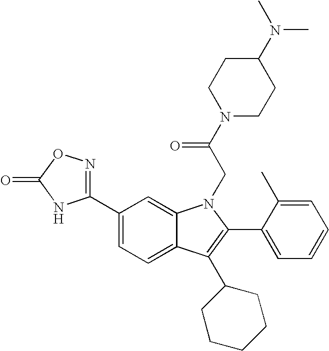 | 542 |
| 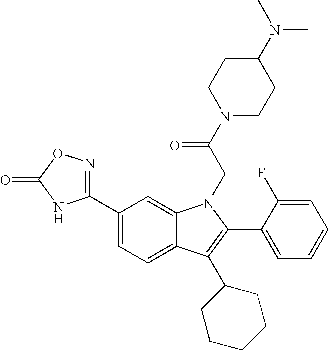 | 546 |
| 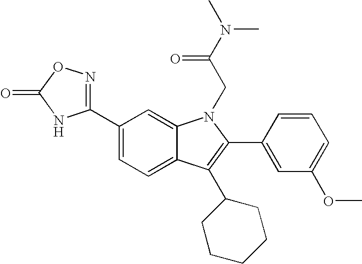 | 475 |
| 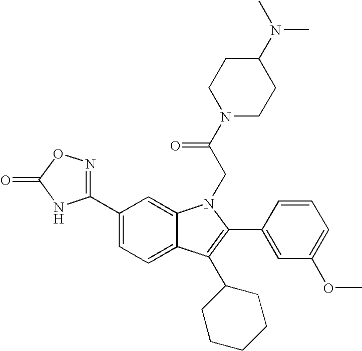 | 558 |
| 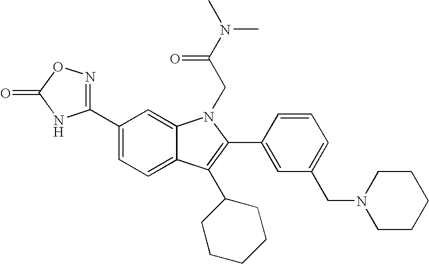 | 542 |
| 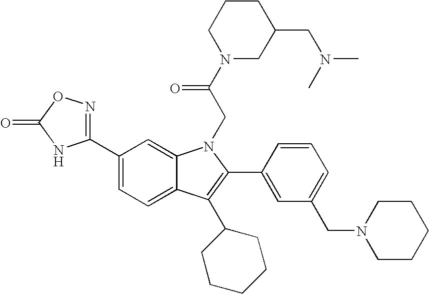 | 639 |
| 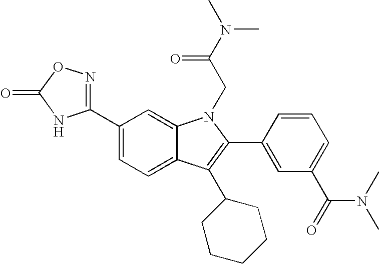 | 516 |
| 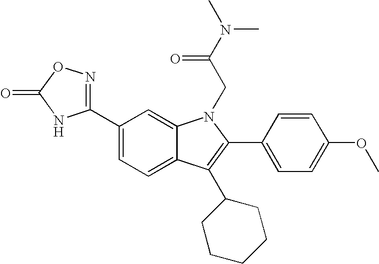 | 475 |
| 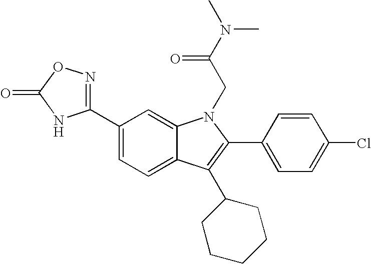 | 479 |
| 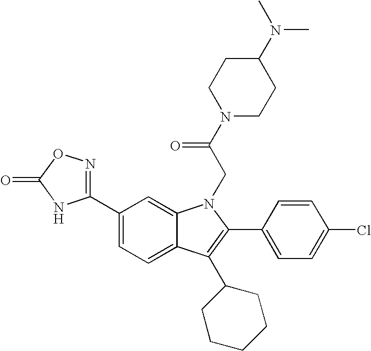 | 562 |
| 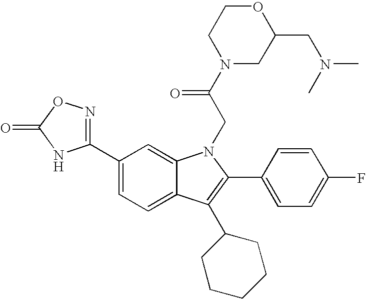 | 562 |
| 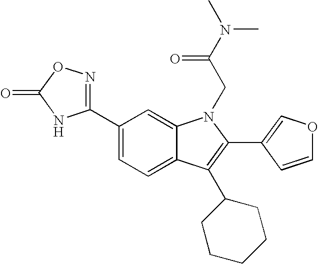 | 435 |
| 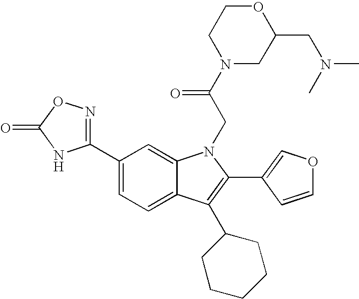 | 534 |
| 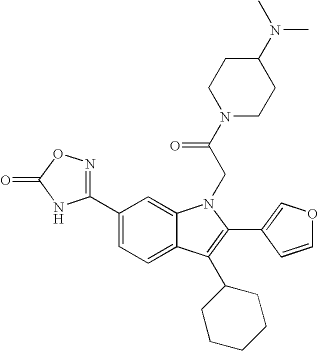 | 518 |
| 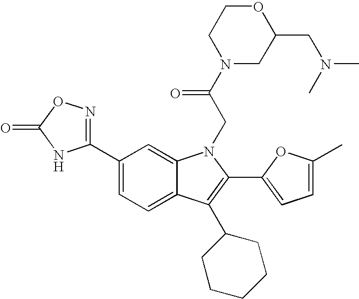 | 548 |
| 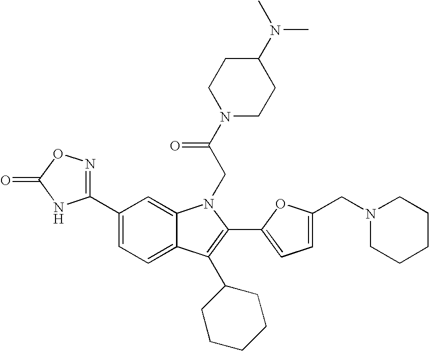 | 615 |
| 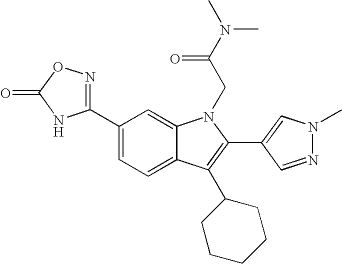 | 449 |
| 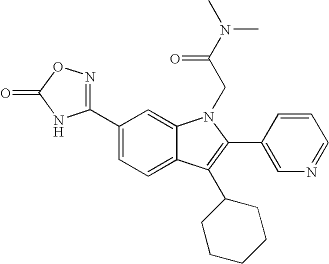 | 446 |
| 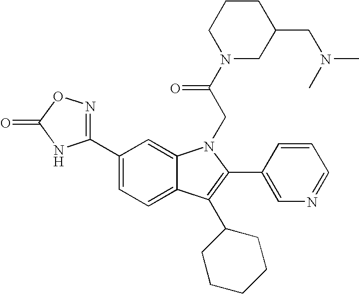 | 543 |
| 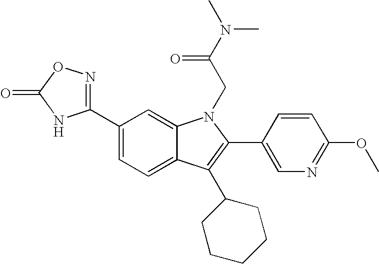 | 476 |
| 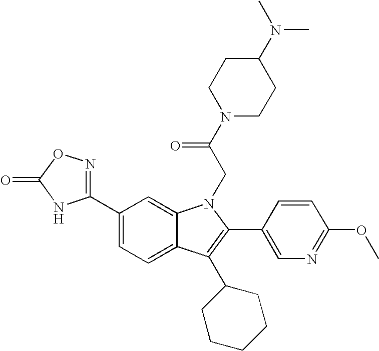 | 559 |
| 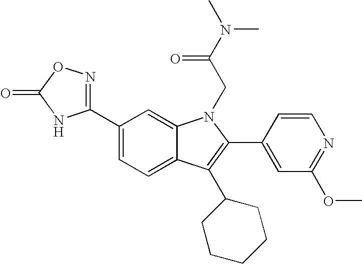 | 476 |
| 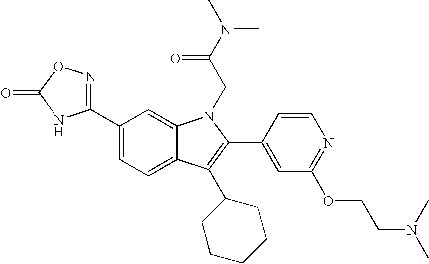 | 533 |
| 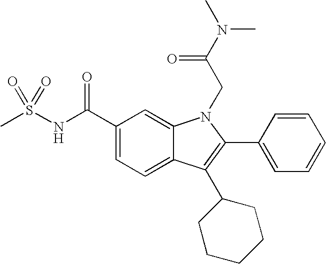 | 482 |
| 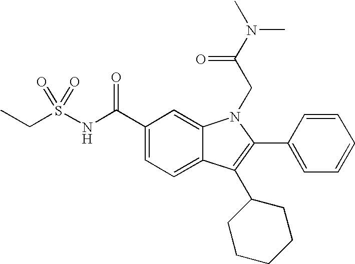 | 496 |
| 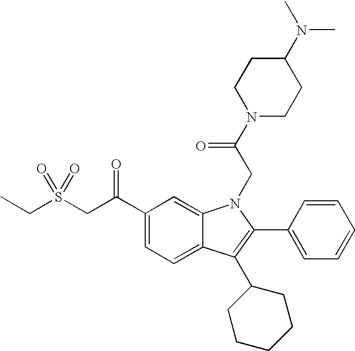 | 579 |
| 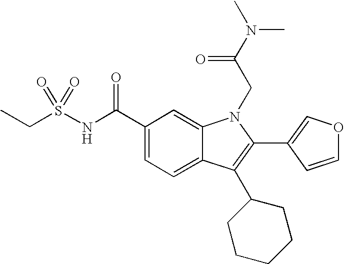 | 486 |
| 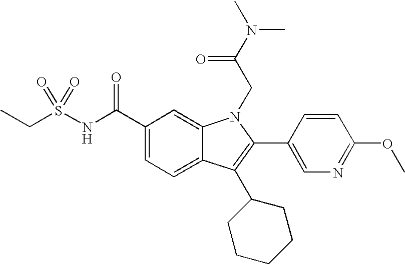 | 527 |
| 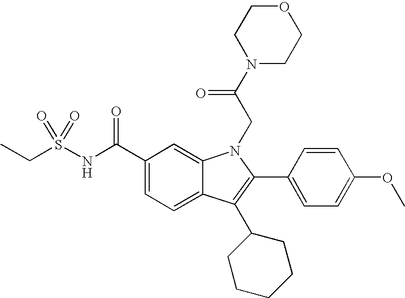 | 568 |
| 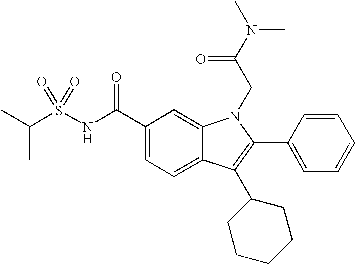 | 510 |
| 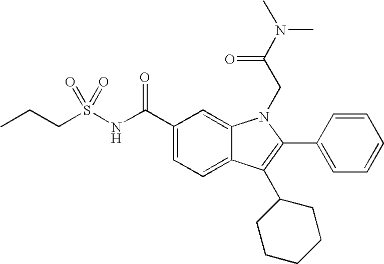 | 510 |
| 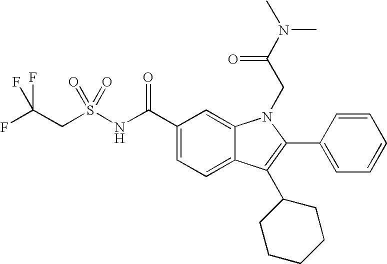 | 550 |
| 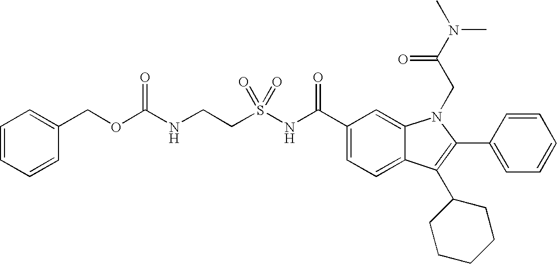 | 645 |
| 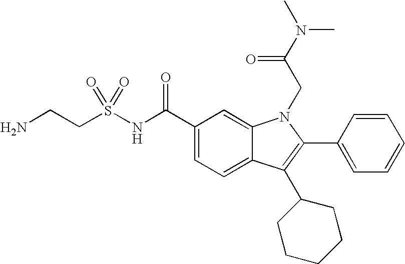 | 511 |
| 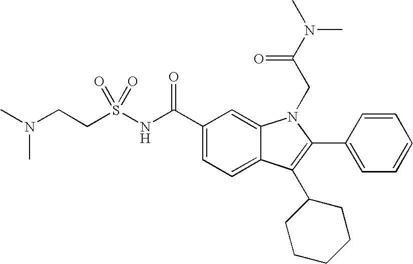 | 539 |
| 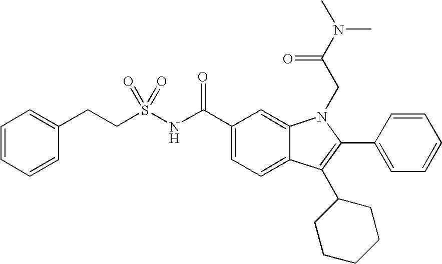 | 572 |
| 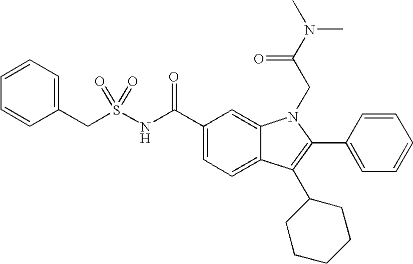 | 558 |
| 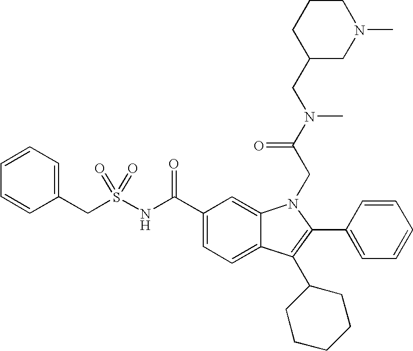 | 655 |
| 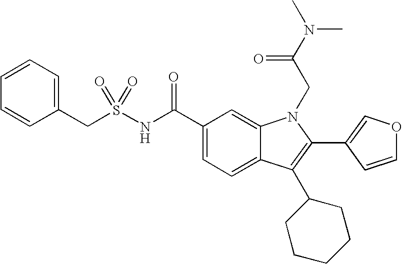 | 548 |
| 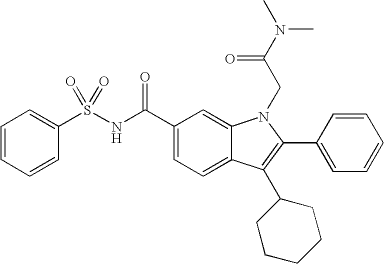 | 544 |
| 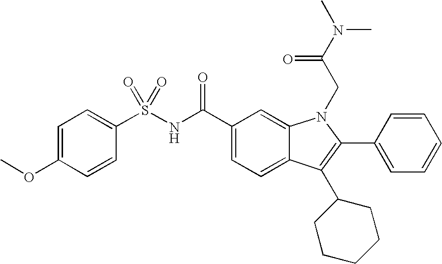 | 574 |
| 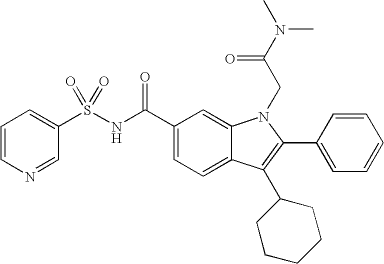 | 545 |
| 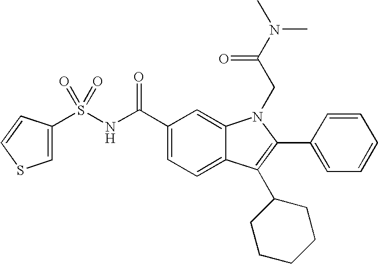 | 550 |

1. A compound of formula (I): 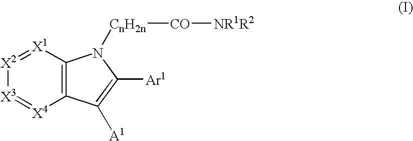
wherein
Ar¹ is a moiety containing at least one aromatic ring and possesses 5-, 6-, 9- or 10-ring atoms optionally containing 1, 2 or 3 heteroatoms independently selected from N, O and S, which ring is optionally substituted at any substitutable position by groups Q¹ and Q²;
Q¹ is halogen, hydroxy, C₁₋₄ alkyl, C₁₋₄ alkoxy, aryl, heteroaryl, CONR^{c}R^{d}, CₘH₂ₘNR^{c}R^{d}, —O—(CH₂)₂₋₄NR^{c}R^{d}, —O—CₘH₂ₘCONR^{c}R^{d}, —O—CₘH₂ₘ aryl, —O—CₘH₂ₘ heteroaryl, —O—CHR^{e}R^{f};
R^{c} and R^{d} are each independently selected from hydrogen, C₁₋₄ alkyl and C(O)C₁₋₄ alkyl;
or R^{c}, R^{d} and the nitrogen atom to which they are attached form a heteroaliphatic ring of 4 to 7 ring atoms, where said ring is optionally substituted by halogen, hydroxy, C₁₋₄ alkyl or C₁₋₄ alkoxy;
m is 0, 1, 2 or 3
R^{e} and R^{f} are each independently selected from hydrogen and C₁₋₄ alkoxy;
or R^{e} and R^{f} are linked by a heteroatom selected from N, O and S to form a heteroaliphatic ring of 4 to 7 ring atoms, where said ring is optionally substituted by halogen, hydroxy, C₁₋₄ alkyl or C₁₋₄alkoxy;
and wherein said C₁₋₄ alkyl, C₁₋₄ alkoxy and aryl groups are optionally substituted by halogen or hydroxy;
Q² is halogen, hydroxy, C₁₋₄ alkyl or C₁₋₄ alkoxy, where said C₁₋₄ alkyl and C₁₋₄ alkoxy groups are optionally substituted by halogen or hydroxyl;
or Q¹ and Q² may be linked by a bond or a heteroatom selected from N, O and S to form a ring of 4 to 7 atoms, where said ring is optionally substituted by halogen, hydroxy, C₁₋₄ alkyl or C₁₋₄ alkoxy;
A¹ is C₁₋₆ alkyl, C₂₋₆ alkenyl, where said C₁₋₆ alkyl and C₂₋₆ alkenyl groups are optionally substituted by C₁₋₄ alkoxy or up to 5 fluorine atoms, or a non-aromatic ring of 3 to 8 ring atoms where said ring may contain a double bond and/or may contain a O, S, SO, SO₂ or NH moiety and where said ring is optionally substituted by one or two alkyl groups of up to 2 carbon atoms or by 1 to 8 fluorine atoms, or a non-aromatic bicyclic moiety of 4 to 8 ring atoms which ring may be optionally substituted by fluorine or hydroxy;
X¹ is N or CR^{a};
X² is N or CR³;
X³ is N or CR⁴;
X⁴ is N or CR^{b};
with the proviso that X² and X³ are not both N;
R^{a} and R^{b} are each independently selected from hydrogen, fluorine, chlorine, C₁₋₄ alkyl, C₂₋₄ alkenyl or C₁₋₄ alkoxy, where said C₁₋₄ alkyl, C₂₋₄ alkenyl and C₁₋₄ alkoxy groups ar optionally substituted by hydroxy or fluorine;
one of R³ or R⁴is hydrogen, halogen, C₁₋₄ alkyl, C₁₋₄ alkoxy, CN, CO₂H, CO₂C₁₋₄ alkyl, aryl, heteroaryl or C(O)NR⁹R¹⁰, where said C₁₋₄ alkyl, C₁₋₄ alkoxy, aryl and heteroaryl groups are optionally substituted by hydroxy or fluorine;
R⁹ is hydrogen or C₁₋₄ alkyl;
R¹⁰ is hydrogen, C₁₋₄ alkyl, C₂₋₄ alkenyl or (CH₂)₀₋₃R¹² or SO₂R¹¹;
R¹² is NR^{h}Rⁱ, OR^{h}, aryl, heteroaryl, indolyl or Het;
R^{h} and Rⁱ are each independently selected from hydrogen and C₁₋₄ alkyl;
Het is a heteroaliphatic ring of 4 to 7 ring atoms, which ring may contain 1, 2 or 3 heteroatoms selected from N, O or S or a group S(O), S(O)₂, NH or NC₁₋₄ alkyl;
R¹¹ is C₁₋₄ alkyl, C₂₋₄ alkenyl or (CH₂)₀₋₃R¹³;
R¹³ is aryl, heteroaryl, C₁₋₄ alkyl, C₃₋₈ cycloalkyl, Het or NR^{m}Rⁿ, wherein Het is as hereinbefore defined, R^{m} and Rⁿ are each independently selected from hydrogen, C₁₋₄ alkyl and CO₂(CH₂)₀₋₃aryl, and wherein R¹³ is optionally substituted by halogen, C₁₋₄ alkyl or NR^{o}R^{p}, wherein R^{o} and R^{p} are each independently selected from hydrogen and C₁₋₄ alkyl; and where R¹⁰ is optionally substituted by hydroxy, fluorine, chlorine, C₁₋₄ alkyl, ═O, CO₂H or CO₂C₁₋₄ alkyl;
or R⁹,R¹⁰ and the nitrogen atom to which they are attached form a heteroaliphatic ring of 4 to 7 ring atoms, where said ring is optionally substituted by halogen, hydroxy, ═O, C₁₋₄ alkyl or C₁₋₄ alkoxy;
the other of R³ and R⁴is hydrogen, fluorine, chlorine, C₁₋₄ alkyl, C₂₋₄ alkenyl or C₁₋₄ alkoxy, where said C₁₋₄ alkyl, C₂₋₄ alkenyl and C₁₋₄ alkoxy groups are optionally substituted by hydroxy or fluorine;
n is 1, 2, 3 or 4;
R¹ and R² are each independently selected from hydrogen, C₁₋₆ alkyl, C₂₋₆ alkenyl, C₁₋₆ alkynyl, C₁₋₄ alkoxy, C₃₋₈ cycloalkyl C₁₋₄ alkyl, (CH₂)₀₋₃R¹⁴;
R¹⁴ is aryl, heteroaryl, NR^{q}R^{r}, Het, where Het is as hereinbefore defined;
R^{q} and R^{r} are each independently selected from hydrogen and C₁₋₄ alkyl;
or R^{q}, R^{r} and the nitrogen atom to which they are attached form a heteroaliphatic ring of 4 to 7 ring atoms;
and R¹ and R² are optionally substituted by hydroxy, C₁₋₄ alkyl, ═O, C(O)C₁₋₄ alkyl or C₃₋₈ cycloalkyl;
or R¹, R² and the nitrogen atom to which they are attached form a heteroaliphatic ring of 4 to 7 ring atoms, which ring optionally contains 1, 2 or 3 additional heteroatoms selected from O and S or a group S(O), S(O)₂, NH or NR^{s}, where R^{s} is C₁₋₄ alkyl or heteroaryl, or said heteroaliphatic ring is fused to or substituted by a spiro-fused five- or six-membered nitrogen-containing heteroaliphatic ring, which heteroaliphatic ring is optionally substituted by hydroxy, C₁₋₄ alkyl, C₁₋₄ alkoxy, (CH₂)₀₋₃NR^{t}R^{u}, aryl, heteroaryl, or a —CH₂— or —CH₂CH₂— alkylene bridge, where aryl and heteroaryl are optionally substituted by hydroxy, C₁₋₄ alkyl or C₁₋₄ alkoxy;
R^{t} and R^{u} are each independently selected from hydrogen, C₁₋₄ alkyl and C(O)C₁₋₄ alkyl,
or R^{t}, R^{u} and the nitrogen atom to which they are attached form a heteroaliphatic ring of 4 to 7 ring atoms optionally substituted by C₁₋₄ alkyl; or a pharmaceutically acceptable salt thereof.

2. A compound of formula (Ia): 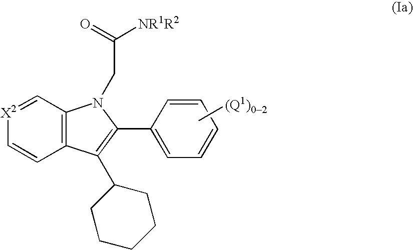
wherein Q¹, X², R¹and R² are as defined in claim 1, or a pharmaceutically acceptable salt thereof.

3. A compound as claimed in claim 1 selected from:
3-cyclohexyl-1-[2-(dimethylamino)-2-oxoethyl]-2-(4-methylphenyl)-1H-indole-6-carboxylic acid,
3-cyclohexyl-1-[2-(dimethylamino)-2-oxoethyl]-2-(2-fluorophenyl)-1H-indole-6-carboxylic acid,
3-cyclohexyl-1-[2-(dimethylamino)-2-oxoethyl]-2-(3-methylphenyl)-1H-indole-6-carboxylic acid,
3-cyclohexyl-1-[2-(dimethylamino)-2-oxoethyl]-2-(2-hydroxypyrimidin-5-yl)-1H-indole-6-carboxylic acid,
3-cyclohexyl-1-[2-(dimethylamino)-2-oxoethyl]-2-(3-furyl)-1H-indole-6-carboxylic acid,
3-{6-carboxy-3-cyclohexyl-1-[2-(dimethylamino)-2-oxoethyl]-1H-indol-2-yl}pyridinium trifluoroacetate,
3-cyclohexyl-1-[2-(dimethylamino)-2-oxoethyl]-2-phenyl-1H-indole-6-carboxylic acid,
3-cyclohexyl-1-[2-(methylamino)-2-oxoethyl]-2-phenyl-1H-indole-6-carboxylic acid,
3-cyclohexyl-1-(2-morpholin-4-yl-2-oxoethyl)-2-phenyl-1H-indole-6-carboxylic acid,
3-cyclohexyl-1-(2-{[(1-methylpyrrolidin-3-yl)methyl]amino}-2-oxoethyl)-2-phenyl-1H-indole-6-carboxylic acid hydrochloride,
3-cyclohexyl-1-[2-(4-methylpiperazin-1-yl)-2-oxoethyl]-2-phenyl-1H-indole-6-carboxylic acid trifluoroacetate,
3-cyclohexyl-1-(2-{[1-(5-methyl-4H-1,2,4-triazol-3-yl)ethyl]amino}-2-oxoethyl)-2-phenyl-1H-indole-6-carboxylic acid trifluoroacetate,
3-cyclohexyl-1-(2-{methyl[(1-methylpiperidin-3-yl)methyl]amino}-2-oxoethyl)-2-phenyl-1H-indole-6-carboxylic acid trifluoroacetate,
3-cyclohexyl-1-(2-{[(1-methylpiperidin-3-yl)methyl]amino}-2-oxoethyl)-2-phenyl-1H-indole-6-carboxylic acid trifluoroacetate,
3-cyclohexyl-1-(2-{methyl[(1-methylpiperidin-2-yl)methyl]amino}-2-oxoethyl)-2-phenyl-1H-indole-6-carboxylic acid trifluoroacetate,
3-cyclohexyl-1-(2-{methyl[(5-methyl-1H-imidazol-2-yl)methyl]amino}-2-oxoethyl)-2-phenyl-1H-indole-6-carboxylic acid trifluoroacetate,
3-cyclohexyl-1-(2-{[2-(dimethylamino)ethyl]amino}-2-oxoethyl)-2-phenyl-1H-indole-6-carboxylic acid trifluoroacetate,
3-cyclohexyl-1-(2-{[2-(1-methylpyrrolidin-3-yl)ethyl]amino}-2-oxoethyl)-2-phenyl-1H-indole-6-carboxylic acid trifluoroacetate,
2-[3-cyclohexyl-2-phenyl-6-(1H-tetrazol-5-yl)-1H-indol-1-yl]-N,N-dimethylacetamide,
3-cyclohexyl-N-methyl-1-(2-morpholin-4-yl-2-oxoethyl)-2-phenyl-1H-indole-6-carboxamide,
3-cyclohexyl-1-[2-(dimethylamino)-2-oxoethyl]-2-phenyl-1H-pyrrolo[2,3-b]pyridine-6-carboxylic acid,
3-cyclohexyl-1-[2-(4-methylpiperazin-1-yl)-2-oxoethyl]-2-phenyl-1H-pyrrolo[2,3-b]pyridine-5-carboxylic acid,
3-cyclohexyl-2-{3-[2-(dimethylamino)ethyl]phenyl}-1-[2-(dimethylamino)-2-oxoethyl]-1H-indole-6-carboxylic acid,
3-cyclohexyl-1-[2-(dimethylamino)prop-2-en-1-yl]-2-(2-methyl-1,2,3,4-tetrahydroisoquinolin-7-yl)-1H-indole-6-carboxylic acid,
2-[3-cyclohexyl-6-(5-oxo-4,5-dihydro-1,2,4-oxadiazol-3-yl)-2-phenyl-1H-indol-1-yl]-N,N-dimethylacetamide,
3-[3-cyclohexyl-1-{2-[4-(dimethylamino)piperidin-1-yl]-2-oxoethyl}-2-(3-furyl)-1H-indol-6-yl]-1,2,4-oxadiazol-5(4H)-one,
3-cyclohexyl-1-[2-(dimethylamino)-2-oxoethyl]-N-(ethylsulfonyl)-2-phenyl-1H-indole-6-carboxamide,
N-(benzylsulfonyl)-3-cyclohexyl-1-[2-(dimethylamino)-2-oxoethyl]-2-phenyl-1H-indole-6-carboxamide,
2-(4-chlorophenyl)-3-cyclohexyl-1-(2-morpholin-4-yl-2-oxoethyl)-1H-indole-6-carboxylic acid,
3-cyclohexyl-2-(4-methoxyphenyl)-1-(2-morpholin-4-yl-2-oxoethyl)-1H-indole-6-carboxylic acid,
1-{[5-carboxy-3-cyclohexyl-2-(4-methoxyphenyl)-1H-indol-1-yl]acetyl}-N,N-dimethylpiperidin-4-aminium trifluoroacetate,
1-{[5-carboxy-3-cyclohexyl-2-(3-furyl)-1H-indol-1-yl]acetyl}-N,N-dimethylpiperidin-4-aminium trifluoroacetate,
(4-{[6-carboxy-2-(4-chlorophenyl)-3-cyclohexyl-1H-indol-1-yl]acetyl}morpholin-2-yl)-N,N-dimethylmethanaminium trifluoroacetate,
1-{2-[benzyl(methyl)amino]-2-oxoethyl}-3-cyclohexyl-2-phenyl-1H-indole-6-carboxylic acid,
1-(2-amino-2-oxoethyl)-3-cyclohexyl-2-phenyl-1H-indole-6-carboxylic acid,
3-cyclohexyl-1-[2-(7-methyl-2,7-diazaspiro[4.4]non-2-yl)-2-oxoethyl]-2-phenyl-1H-indole-6-carboxylic acid,
1-[2-(benzylamino)-2-oxoethyl]-3-cyclohexyl-2-phenyl-1H-indole-6-carboxylic acid,
3-cyclohexyl-1-(2-{[(3R,4R)-4-hydroxy-1,1-dioxidotetrahydro-3-thienyl]amino}-2-oxoethyl)-2-phenyl-1H-indole-6-carboxylic acid,
3-cyclohexyl-1-[2-oxo-2-(3-pyridin-3-ylpyrrolidin-1-yl)ethyl]-2-phenyl-1H-indole-6-carboxylic acid,
3-cyclohexyl-1-(2-{methyl[1-(1,3-thiazol-2-yl)ethyl]amino}-2-oxoethyl)-2-phenyl-1H-indole-6-carboxylic acid,
3-cyclohexyl-1-{2-[4-(4-methyl-4H-1,2,4-triazol-3-yl)piperidin-1-yl]-2-oxoethyl}-2-phenyl-1H-indole-6-carboxylic acid,
3-cyclohexyl-1-{2-[4-(6-methoxypyridin-2-yl)piperazin-1-yl]-2-oxoethyl}-2-phenyl-1H-indole-6-carboxylic acid,
3-cyclohexyl-1-[2-(dimethylamino)-2-oxoethyl]-2-pyridin-4-yl-1H-indole-6-carboxylic acid,
3-cyclohexyl-1-(2-{3-[(dimethylamino)methyl]piperidin-1-yl}-2-oxoethyl)-2-phenyl-1H-indole-6-carboxylic acid,
3-cyclohexyl-1-(2-{2-[2-(dimethylamino)ethyl]piperidin-1-yl}-2-oxoethyl)-2-phenyl-1H-indole-6-carboxylic acid,
(1-pyridin-4-ylethyl)amino]-2-oxoethyl}-2-phenyl-1H-indole-6-carboxylic acid,
3-cyclohexyl-1-(2-oxo-2-{[(1-piperidin-1-ylcyclopentyl)methyl]amino}ethyl)-2-phenyl-1H-indole-6-carboxylic acid,
3-cyclohexyl-1-[2-oxo-2-(2-pyridin-4-ylpyrrolidin-1-yl)ethyl]-2-phenyl-1H-indole-6-carboxylic acid,
3-cyclohexyl-1-{2-[(1S,4S)-5-methyl-2,5-diazabicyclo[2.2.1]hept-2-yl]-2-oxoethyl}-2-phenyl-1H-indole-6-carboxylic acid,
3-cyclohexyl-1-[2-(hexahydropyrrolo[1,2-a]pyrazin-2(1H)-yl)-2-oxoethyl]-2-phenyl-1H-indole-6-carboxylic acid,
3-cyclohexyl-1-(2-{[2-(4-methylpiperazin-1-yl)ethyl]amino}-2-oxoethyl)-2-phenyl-1H-indole-6-carboxylic acid,
3-cyclohexyl-1-{2-[(cyclopropylmethyl)amino]-2-oxoethyl}-2-phenyl-1H-indole-6-carboxylic acid,
3-cyclohexyl-1-[2-oxo-2-(prop-2-yl-1-ylamino)ethyl]-2-phenyl-1H-indole-6-carboxylic acid,
3-cyclohexyl-1-{2-[(2-morpholin-4-ylethyl)amino]-2-oxoethyl}-2-phenyl-1H-indole-6-carboxylic acid,
3-cyclohexyl-1-{2-[methyl(1-methylpiperidin-4-yl)amino]-2-oxoethyl}-2-phenyl-1H-indole-6-carboxylic acid,
3-cyclohexyl-1-(2-{[2-(diisopropylamino)ethyl]amino}-2-oxoethyl)-2-phenyl-1H-indole-6-carboxylic acid,
3-cyclohexyl-1-[2-(dimethylamino)-2-oxoethyl]-2-(3-fluoro-4-hydroxyphenyl)-1H-indole-6-carboxylic acid,
3-cyclohexyl-1-[2-(dimethylamino)-2-oxoethyl]-2-(4-hydroxyphenyl)-1H-indole-6-carboxylic acid,
2-(3-chlorophenyl)-3-cyclohexyl-1-[2-(dimethylamino)-2-oxoethyl]-1H-indole-6-carboxylic acid,
2-(4-chlorophenyl)-3-cyclohexyl-1-[2-(dimethylamino)-2-oxoethyl]-1H-indole-6-carboxylic acid,
3-cyclohexyl-1-[2-(dimethylamino)-2-oxoethyl]-2-(3-fluorophenyl)-1H-indole-6-carboxylic acid,
3-cyclohexyl-1-[2-(dimethylamino)-2-oxoethyl]-2-(4-fluorophenyl)-1H-indole-6-carboxylic acid,
3-cyclohexyl-1-[2-(dimethylamino)-2-oxoethyl]-2-(3-thienyl)-1H-indole-6-carboxylic acid,
2-[4-(aminocarbonyl)phenyl]-3-cyclohexyl-1-[2-(dimethylamino)-2-oxoethyl]-1H-indole-6-carboxylic acid,
2-[3-(acetylamino)phenyl]-3-cyclohexyl-1-[2-(dimethylamino)-2-oxoethyl]-1H-indole-6-carboxylic acid,
3-cyclohexyl-1-[2-(dimethylamino)-2-oxoethyl]-2-[3-(1H-pyrazol-1-yl)phenyl]-1H-indole-6-carboxylic acid,
3-cyclohexyl-1-[2-(dimethylamino)-2-oxoethyl]-2-(3-hydroxyphenyl)-1H-indole-6-carboxylic acid,
3-cyclohexyl-1-[2-(dimethylamino)-2-oxoethyl]-2-(2-methylphenyl)-1H-indole-6-carboxylic acid,
3-cyclohexyl-2-(3,5-difluorophenyl)-1-[2-(dimethylamino)-2-oxoethyl]-1H-indole-6-carboxylic acid,
3-cyclohexyl-2-(3,4-difluorophenyl)-1-[2-(dimethylamino)-2-oxoethyl]-1H-indole-6-carboxylic acid,
3-cyclohexyl-2-(2,4-difluorophenyl)-1-[2-(dimethylamino)-2-oxoethyl]-1H-indole-6-carboxylic acid,
3-cyclohexyl-1-[2-(dimethylamino)-2-oxoethyl]-2-(4-methoxyphenyl)-1H-indole-6-carboxylic acid,
3-cyclohexyl-1-[2-(dimethylamino)-2-oxoethyl]-2-(3-methoxyphenyl)-1H-indole-6-carboxylic acid,
3-cyclohexyl-1-[2-(dimethylamino)-2-oxoethyl]-2-(2-methoxyphenyl)-1H-indole-6-carboxylic acid,
3-cyclohexyl-1-[2-(dimethylamino)-2-oxoethyl]-2-(2-hydroxyphenyl)-1H-indole-6-carboxylic acid,
2-(2-chlorophenyl)-3-cyclohexyl-1-[2-(dimethylamino)-2-oxoethyl]-1H-indole-6-carboxylic acid
3-cyclohexyl-2-(3-fluorophenyl)-1-(2-{methyl[(1-methylpiperidin-3-yl)methyl]amino}-2-oxoethyl)-1H-indole-6-carboxylic acid,
3-cyclohexyl-1-(2-{3-[(dimethylamino)methyl]piperidin-1-yl}-2-oxoethyl)-2-(3-fluorophenyl)-1H-indole-6-carboxylic acid,
3-cyclopentyl-1-{2-[methyl(phenyl)amino]-2-oxoethyl}-2-phenyl-1H-indole-6-carboxylic acid,
3-cyclopentyl-1-[2-oxo-2-(4-pyrrolidin-1-ylpiperidin-1-yl)ethyl]-2-phenyl-1H-indole-6-carboxylic acid,
3-cyclohexyl-1-[2-oxo-2-(4-pyrrolidin-1-ylpiperidin-1-yl)ethyl]-2-pyridin-4-yl-1H-indole-6-carboxylic acid,
1-(2-{[(1-acetylpyrrolidin-2-yl)methyl]amino}-2-oxoethyl)-3-cyclohexyl-2-pyridin-4-yl-1H-indole-6-carboxylic acid,
3-cyclohexyl-1-{2-[3-(dimethylamino)piperidin-1-yl]-2-oxoethyl}-2-pyridin-3-yl-1H-indole-6-carboxylic acid,
3-cyclohexyl-1-{2-[[2-(dimethylamino)-2-oxyethyl](methyl)amino]-2-oxoethyl}-2-pyridin-3-yl-1H-indole-6-carboxylic acid,
3-cyclohexyl-1-[2-(hexahydropyrrolo[1,2-a]pyrazin-2(1H)-yl)-2-oxoethyl]-2-pyridin-3-yl-1H-indole-6-carboxylic acid,
3-cyclopentyl-1-(2-{methyl[(1-methylpiperidin-4-yl)methyl]amino}-2-oxoethyl)-2-phenyl-1H-indole-6-carboxylic acid,
3-cyclopentyl-1-(2-{[(1-ethyl-5-oxopyrrolidin-3-yl)methyl]amino}-2-oxoethyl)-2-phenyl-1H-indole-6-carboxylic acid,
3-cyclohexyl-2-{4-[2-(dimethylamino)-2-oxoethoxy]phenyl}-1-{2-[methyl(pyrazin-2-ylmethyl)amino]-2-oxoethyl}-1H-indole-6-carboxylic acid,
2-(4-chloro-2-fluorophenyl)-3-cyclohexyl-1-(2-morpholin-4-yl-2-oxoethyl)-1H-indole-6-carboxylic acid,
3-cyclohexyl-1-(2-{[(1,1-dioxidotetrahydro-3-thienyl)methyl]amino}-2-oxoethyl)-2-(3-fluorophenyl)-1H-indole-6-carboxylic acid,
2-biphenyl-3-yl-3-cyclohexyl-1-{2-[4-(dimethylamino)piperidin-1-yl]-2-oxoethyl{1H-indole-6-carboxylic acid,
2-(2-chlorophenyl)-3-cyclohexyl-1-{2-[4-(dimethylamino)piperidin-1-yl]-2-oxoethyl}-1H-indole-6-carboxylic acid,
3-cyclohexyl-1-{2-[4-(dimethylamino)piperidin-1-yl]-2-oxoethyl}-2-(5-fluoro-2-methoxyphenyl)-1H-indole-6-carboxylic acid,
3-cyclohexyl-1-{2-[4-(dimethylamino)piperidin-1-yl]-2-oxoethyl}-2-(3-thienyl)-1H-indole-6-carboxylic acid,
2-[4-(benzyloxy)phenyl]-3-cyclohexyl-1-{2-[4-(dimethylamino)piperidin-1-yl]-2-oxoethyl}-1H-indole-6-carboxylic acid,
3-cyclohexyl-1-{2-[4-(dimethylamino)piperidin-1-yl]-2-oxoethyl}-2-(4-isopropoxyphenyl)-1H-indole-6-carboxylic acid,
3-cyclohexyl-1-{2-[4-(dimethylamino)piperidin-1-yl]-2-oxoethyl}-2-[3-(piperidin-1-ylcarbonyl)phenyl]-1H-indole-6-carboxylic acid,
3-cyclohexyl-1-{2-[4-(dimethylamino)piperidin-1-yl]-2-oxoethyl}-2-(3-methylphenyl)-1H-indole-6-carboxylic acid,
3-cyclohexyl-1-[2-(methylamino)-2-oxoethyl]-2-phenyl-1H-indole-5-carboxylic acid,
3-cyclohexyl-1-(2-{methyl [(1-methylpiperidin-3-yl)methyl]amino}-2-oxoethyl)-2-phenyl-1H-indole-5-carboxylic acid,
3-cyclohexyl-1-{2-[[2-(dimethylamino)-2-oxoethyl](methyl)amino]-2-oxoethyl}-2-phenyl-1H-indole-5-carboxylic acid,
1-[2-(2-{[acetyl(methyl)amino]methyl}morpholin-4-yl)-2-oxoethyl]-3-cyclohexyl-2-(3-fluorophenyl)-1H-indole-6-carboxylic acid,
3-cyclopentyl-1-[2-(1,1-dioxidothiomorpholin-4-yl)-2-oxoethyl]-2-phenyl-1H-indole-5-carboxylic acid,
3-cyclopentyl-1-[2-oxo-2-(4-pyrrolidin-1-ylpiperidin-1-yl)ethyl]-2-phenyl-1H-indole-5-carboxylic acid,
3-cyclopentyl-1-{2-[(cyclopropylmethyl)amino]-2-oxoethyl}-2-phenyl-1H-indole-5-carboxylic acid,
3-cyclopentyl-1-(2-{[(1-ethyl-5-oxopyrrolidin-3-yl)methyl]amino}-2-oxoethyl)-2-phenyl-1H-indole-5-carboxylic acid,
3-cyclohexyl-1-{2-[4-(dimethylamino)piperidin-1-yl]-2-oxoethyl}-2-pyrimidin-5-yl-1H-indole-6-carboxylic acid,
2-(4-chlorophenyl)-3-cyclohexyl-1-[2-(4-methyl-1,4-diazepan-1-yl)-2-oxoethyl]-1H-indole-6-carboxylic acid,
2-(4-chlorophenyl)-3-cyclohexyl-1-[2-(4-isopropylpiperazin-1-yl)-2-oxoethyl]-1H-indole-6-carboxylic acid,
2-(4-chlorophenyl)-3-cyclohexyl-1-[2-oxo-2-(3-pyrrolidin-1-ylpiperidin-1-yl)ethyl]-1H-indole-6-carboxylic acid,
2-(4-chlorophenyl)-3-cyclohexyl-1-(2-oxo-2-piperazin-1-ylethyl)-1H-indole-6-carboxylic acid,
3-cyclohexyl-2-(3-furyl)-1-[2-oxo-2-(4-pyrrolidin-1-ylpiperidin-1-yl)ethyl]-1H-indole-6-carboxylic acid,
3-cyclohexyl-1-(2-{2-[(dimethylamino)methyl]morpholin-4-yl}-2-oxoethyl)-2-(3-furyl)-1H-indole-6-carboxylic acid,
1-[2-(4-azetidin-1-ylpiperidin-1-yl)-2-oxoethyl]-3-cyclohexyl-2-(4-methoxyphenyl)-1H-indole-6-carboxylic acid,
3-cyclohexyl-2-(4-methoxyphenyl)-1-[2-oxo-2-(4-pyrrolidin-1-ylpiperidin-1-yl)ethyl]-1H-indole-6-carboxylic acid,
3-cyclohexyl-1-{2-[4-(diethylamino)piperidin-1-yl]-2-oxoethyl}-2-(4-methoxyphenyl)-1H-indole-6-carboxylic acid,
3-cyclohexyl-2-{3-[(dimethylamino)methyl]phenyl}-1-[2-(dimethylamino)-2-oxoethyl]-1H-indole-6-carboxylic acid,
3-cyclohexyl-1-[2-(dimethylamino)-2-oxoethyl]-2-{3-[(1-methylpiperidin-4-yl)oxy]phenyl}-1H-indole-6-carboxylic acid,
3-cyclohexyl-1-[2-(dimethylamino)-2-oxoethyl]-2-phenyl-1H-pyrrolo[3,2-b]pyridine-6-carboxylic acid,
3-cyclohexyl-1-[2-(dimethylamino)-2-oxoethyl]-2-(1-naphthyl)-1H-indole-6-carboxylic acid,
3-cyclohexyl-1-[2-(dimethylamino)-2-oxoethyl]-2-(2-naphthyl)-1H-indole-6-carboxylic acid,
3-cyclohexyl-1-(2-morpholin-4-yl-2-oxoethyl)-1H, 1′H-2,5′-bisindole-6-carboxylic acid,
3-cyclohexyl-1-[2-(dimethylamino)-2-oxoethyl]-2-(8-methylquinolin-4-yl)-1H-indole-6-carboxylic acid,
3-cyclohexyl-N-methyl-1-[2-(4-methylpiperazin-1-yl)-2-oxoethyl]-2-phenyl-1H-indole-6-carboxamide,
3-cyclohexyl-N-[(4-methyl-1H-imidazol-2-yl)methyl]-1-(2-morpholin-4-yl-2-oxoethyl)-2-phenyl-1H-indole-6-carboxamide,
3-cyclohexyl-1-(2-morpholin-4-yl-2-oxoethyl)-2-phenyl-1H-indole-6-carboxamide,
3-cyclohexyl-N,N-dimethyl-1-(2-morpholin-4-yl-2-oxoethyl)-2-phenyl-1H-indole-6-carboxamide,
3-cyclohexyl-N-isopropyl-1-(2-morpholin-4-yl-2-oxoethyl)-2-phenyl-1H-indole-6-carboxamide,
N-allyl-3-cyclohexyl-1-(2-morpholin-4-yl-2-oxoethyl)-2-phenyl-1H-indole-6-carboxamide,
3-cyclohexyl-N-[2-(dimethylamino)ethyl]-1-(2-morpholin-4-yl-2-oxoethyl)-2-phenyl-1H-indole-6-carboxamide,
3-cyclohexyl-N-[(1-methylpiperidin-3-yl)methyl]-1-(2-morpholin-4-yl-2-oxoethyl)-2-phenyl-1H-indole-6-carboxamide,
3-cyclohexyl-N-[(1-methylpyrrolidin-3-yl)methyl]-1-(2-morpholin-4-yl-2-oxoethyl)-2-phenyl-1H-indole-6-carboxamide,
3-cyclohexyl-6-[(4-methylpiperazin-1-yl)carbonyl]-1-(2-morpholin-4-yl-2-oxoethyl)-2-phenyl-1H-indole,
3-cyclohexyl-1-(2-morpholin-4-yl-2-oxoethyl)-2-phenyl-N-(tetrahydrofuran-3-yl)-1H-indole-6-carboxamide,
3-cyclohexyl-N-(1,1-dioxidotetrahydro-3-thienyl)-1-(2-morpholin-4-yl-2-oxoethyl)-2-phenyl-1H-indole-6-carboxamide,
3-cyclohexyl-N-(2-furylmethyl)-1-(2-morpholin-4-yl-2-oxoethyl)-2-phenyl-1H-indole-6-carboxamide,
3-cyclohexyl-N-[(6-methylpyridin-2-yl)methyl]-1-(2-morpholin-4-yl-2-oxoethyl)-2-phenyl-1H-indole-6-carboxamide,
3-cyclohexyl-[1-(2-morpholin-4-yl-2-oxoethyl)-N,2-diphenyl-1H-indole-6-carboxamide,
N-benzyl-3-cyclohexyl-1-(2-morpholin-4-yl-2-oxoethyl)-2-phenyl-1H-indole-6-carboxamide,
4-{[3-cyclohexyl-1-(2-morpholin-4-yl-2-oxoethyl)-2-phenyl-1H-indol-6-yl]carbonyl}piperazin-2-one,
3-cyclohexyl-N-(2-methoxyethyl)-1-(2-morpholin-4-yl-2-oxoethyl)-2-phenyl-1H-indole-6-carboxamide,
3-cyclohexyl-N-(2-morpholin-4-ylethyl)-1-(2-morpholin-4-yl-2-oxoethyl)-2-phenyl-1H-indole-6-carboxamide,
3-cyclohexyl-N-[2-(1-methylpyrrolidin-3-yl)ethyl]-1-(2-morpholin-4-yl-2-oxoethyl)-2-phenyl-1H-indole-6-carboxamide,
N-{[3-cyclohexyl-1-(2-morpholin-4-yl-2-oxoethyl)-2-phenyl-1H-indol-6-yl]carbonyl}-5-hydroxy-L-tryptophan,
3-cyclohexyl-1-(2-morpholin-4-yl-2-oxoethyl)-2-phenyl-N-[2-(1H-pyrazol-1-yl)ethyl]-1H-indole-6-carboxamide,
3-{3-cyclohexyl-1-[2-(4-methylpiperazin-1-yl)-2-oxoethyl]-2-phenyl-1H-indol-6yl}-1,2,4-oxadiazol-5(4H)-one,
2-[3-cyclohexyl-6-(5-oxo-4,5-dihydro-1,2,4-oxadiazol-3-yl)-2-phenyl-1H-indol-1-yl]-N-methyl-N-[(1-methylpiperidin-3-yl)methyl]acetamide,
2-[3-cyclohexyl-6-(5-oxo-4,5-dihydro-1,2,4-oxadiazol-3-yl)-2-phenyl-1H-indol-1-yl]-N,N-dimethylacetamide,
3-[3-cyclohexyl-1-(2-morpholin-4-yl-2-oxoethyl)-2-phenyl-1H-indol-6-yl]-1,2,4-oxadiazol-5(4H)-one,
2-[3-cyclohexyl-6-(5-oxo-4,5-dihydro-1,2,4-oxadiazol-3-yl)-2-phenyl-1H-indol-1-yl]-N-[(1methylpyrrolidin-3-yl)methyl]acetamide,
3-[3-cyclohexyl-1-{2-[4-(dimethylamino)piperidin-1-yl]-2-oxoethyl}-2-(2-methylphenyl)-1H-indol-6-yl]-1,2,4-oxadiazol-5(4H)-one,
3-[3-cyclohexyl-1-{2-[4-(dimethylamino)piperidin-1-yl]-2-oxoethyl}-2-(2-fluorophenyl)-1H-indol-6-yl]-1,2,4-oxadiazol-5(4H)-one,
2-[3-cyclohexyl-2-(3-methoxyphenyl)-6-(5-oxo-4,5-dihydro-1,2,4-oxadiazol-3-yl)-1H-indol-1-yl]-N,N-dimethylacetamide,
3-[3-cyclohexyl-1-{2-[4-(dimethylamino)piperidin-1-yl]-2-oxoethyl}-2-(3-methoxyphenyl)-1H-indol-6-yl]-1,2,4-oxadiazol-5(4H)-one,
2-{3-cyclohexyl-6-(5-oxo-4,5-dihydro-1,2,4-oxadiazol-3-yl)-2-[3-(piperidin-1-ylmethyl)phenyl]-1H-indol-1-yl }-N,N-dimethylacetamide,
3-{3-cyclohexyl-1-(2-{3-[(dimethylamino)methyl]piperidin-1-yl}-2-oxoethyl)-2-[3-piperidin-1-ylmethyl)phenyl]-1H-indol-6-yl }-1,2,4-oxadiazol-5(4H)-one,
3-[3-cyclohexyl-1-[2-(dimethylamino)-2-oxoethyl]-6-(5-oxo-4,5-dihydro-1,2,4-oxadiazol-3-yl)-1H-indol-2-yl]-N,N-dimethylbenzamide,
2-[3-cyclohexyl-2-(4-methoxyphenyl)-6-(5-oxo-4,5-dihydro-1,2,4-oxadiazol-3-yl)-1H-indol-1-yl]-N,N-dimethylacetamide,
2-[2-(4-chlorophenyl)-3-cyclohexyl-6-(5-oxo-4,5-dihydro-1,2,4-oxadiazol-3-yl)-1H-indol-1-yl]-N,N-dimethylacetamide,
3-(2-(4-chlorophenyl)-3-cyclohexyl-1-{2-[4-(dimethylamino)piperidin-1-yl]-2-oxoethyl}-1H-indol-6-yl)-1,2,4-oxadiazol-5(4H)-one,
3-[3-cyclohexyl-1-(2-{2-[(dimethylamino)methyl]morpholin-4-yl}-2-oxoethyl)-2-(4-fluorophenyl)-1H-indol-6-yl]-1,2,4-oxadiazol-5(4H)-one,
2-[3-cyclohexyl-2-(3-furyl)-6-(5-oxo-4,5-dihydro-1,2,4-oxadiazol-3-yl)-1H-indol-1yl]-N,N-dimethylacetamide,
3-[3-cyclohexyl-1-(2-{2-[(dimethylamino)methylmorpholine-4-yl }-2-oxoethyl)-2-(3-furyl)-1H-indol-6-yl]-1,2,4-oxadiazol-5(4H)-one,
3-[3-cyclohexyl-1-{2-[4-(dimethylamino)piperidin-1-yl]-2-oxoethyl}-2-(3-furyl)-1H-indol-6-yl]-1,2,4-oxadiazol-5(4H)-one,
3-[3-cyclohexyl-1-(2-{2-[(dimethylamino)methyl]morpholin-4-yl}-2-oxoethyl)-2-(5-methyl-2-furyl)-1H-indol-6-yl]-1,2,4-oxadiazol-5(4H)-one,
3-{3-cyclohexyl-1-{2-[4-(dimethylamino)piperidin-1-yl]-2-oxoethyl}-2-[5-piperidin-1-ylmethyl)-2-furyl]-1H-indol-6-yl}-1,2,4-oxadiazol-5(4H)-one,
2-[3-cyclohexyl-2-(1-methyl-1H-pyrazol-4-yl)-6-(5-oxo-4,5-dihydro-1,2,4-oxadiazol-3-yl)-1H-indol-1-yl]-N,N-dimethylacetamide,
2-[3-cyclohexyl-6-(5-oxo-4,5-dihydro-1,2,4-oxadiazol-3-yl)-2-pyridin-3-yl-1H-indol-1-yl]-N,N-dimethylacetamide,
3-[3-cyclohexyl-1-(2-{3-[(dimethylamino)methyl]piperidin-1-yl}-2-oxoethyl)-2-pyridin-3yl-1H-indol-6-yl]-1,2,4-oxadiazol-5(4H)-one,
2-[3-cyclohexyl-2-(6-methoxypyridin-3-yl)-6-(5-oxo-4,5-dihydro-1,2,4-oxadiazol-3-yl)-1H-indol-1-yl]-N,N-dimethylacetamide,
3-[3-cyclohexyl-1-{2-[4-(dimethylamino)piperidin-1-yl]-2-oxoethyl}-2-(6-methoxypyridin-3-yl)-1H-indol-6-yl]-1,2,4-oxadiazol-5(4H)-one, 2-[3-cyclohexyl-2-(2-methoxypyridin-4-yl)-6-(5-oxo-4,5-dihydro-1,2,4-oxadiazol-3-yl)-1H-indol-1-yl]-N,N-dimethylacetamide,
2-[3-cyclohexyl-2-{2-[2-(dimethylamino)ethoxy]pyridin-4-yl }-6-(5-oxo-4,5-dihydro-1,2,4-oxadiazol-3-yl)-1H-indol-1-yl]-N,N-dimethylacetamide,
3-cyclohexyl-1-[2-(dimethylamino)-2-oxoethyl]-N-(methylsulfonyl)-2-phenyl-1H-indole-6-carboxamide,
3-cyclohexyl-1-[2-(dimethylamino)-2-oxoethyl]-N-(ethylsulfonyl)-2-phenyl-1H-indole-6-carboxamide,
3-cyclohexyl-1-[2-(dimethylamino)-2-oxoethyl]-N-(ethylsulfonyl)-2-(3-furyl)-1H-indole-6-carboxamide,
3-cyclohexyl-1-[2-(dimethylamino)-2-oxoethyl]-N-(ethylsulfonyl)-2-(6-methoxypyridin-3-yl)-1H-indole-6-carboxamide,
3-cyclohexyl-N-(ethylsulfonyl)-2-(4-methoxyphenyl)-1-(2-morpholin4-yl-2-oxoethyl)-1H-indole-6-carboxamide,
3-cyclohexyl-1-[2-(dimethylamino)-2-oxoethyl]-N-(isopropylsulfonyl)-2-phenyl-1H-indole-6-carboxamide,
3-cyclohexyl-1-[2-(dimethylamino)-2-oxoethyl]-2-phenyl-N-(propylsulfonyl)-1H-indole-6-carboxamide,
3-cyclohexyl-1-[2-(dimethylamino)-2-oxoethyl]-2-phenyl-N-[(2,2,2-trifluoroethyl)sulfonyl]-1H-indole-6-carboxamide,
benzyl (2-{[({3-cyclohexyl-1-[2-(dimethylamino)-2-oxoethyl]-2-phenyl-1H-indol-6-yl}carbonyl)amino]sulfonyl}ethyl)carbamate,
N-[(2-aminoethyl)sulfonyl]-3-cyclohexyl-1-[2-(dimethylamino)-2-oxoethyl]-2-phenyl-1H-indole-6-carboxamide,
3-cyclohexyl-N-{[2-(dimethylamino)ethyl]sulfonyl}-1-[2-(dimethylamino)-2-oxoethyl]-2-phenyl-1H-indole-6-carboxamide,
3-cyclohexyl-1-[2-(dimethylamino)-2-oxoethyl]-2-phenyl-N-[(2-phenylethyl)sulfonyl]-1H-indole-6-carboxamide,
N-(benzylsulfonyl)-3-cyclohexyl-1-[2-(dimethylamino)-2-oxoethyl]-2-phenyl-1H-indole-6-carboxamide,
N-(benzylsulfonyl)-3-cyclohexyl-1-(2-{methyl[(1-methylpiperidin-3-yl)methyl]amino}-2-oxoethyl)-2-phenyl-1H-indole-6-carboxamide,
N-(benzylsulfonyl)-3-cyclohexyl-1-[2-(dimethylamino)-2-oxoethyl]-2-(3-furyl)-1H-indole-6-carboxamide,
3-cyclohexyl-1-[2-(dimethylamino)-2-oxoethyl]-2-phenyl-N-(phenylsulfonyl)-1H-indole-6-carboxamide,
3-cyclohexyl-1-[2-(dimethylamino)-2-oxoethyl]-N-[(4-methoxyphenyl)sulfonyl]-2-phenyl-1H-indole-6-carboxamide,
3-cyclohexyl-1-[2-(dimethylamino)-2-oxoethyl]-2-phenyl-N-(pyridin-3-ylsulfonyl)-1H-indole-6-carboxamide,
3-cyclohexyl-1-[2-(dimethylamino)-2-oxoethyl]-2-phenyl-N-(3-thienylsulfonyl)-1H-indole-6-carboxamide,
or a pharmaceutically acceptable salt thereof.

4. (canceled)

5. A pharmaceutical composition comprising a compound as claimed in claim 1 or a pharmaceutically acceptable salt thereof, in association with a pharmaceutically acceptable carrier.

6. A method for the treatment or prevention of illness due to hepatitis C virus, which method comprises administration to a subject suffering from the condition a compound as claimed in claim 1 or a pharmaceutically acceptable salt thereof.

7. (canceled)

8. A process for the preparation of a compound as claimed in claim 1 which comprises: either
(A) reacting a compound of formula (II) with a compound of formula (III): 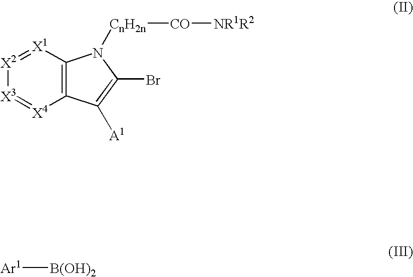
wherein X¹, X², X³, X⁴, R¹, R², A¹ and Ar¹ are as defined in claim 1, in the presence of a Pd(0) catalyst under conditions typical for the Suzuki reaction;
(B) reacting a compound of formula (IV) with a compound of formula (V): 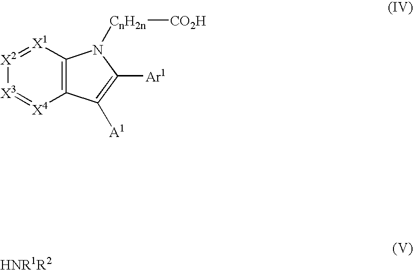
wherein X¹, X², X³, X⁴, A¹, Ar¹, n, R¹ and R² are as defined in claim 1, in the presence of a coupling reagent and a base;
(C) where X² is CR³ and R³ is C(O)NR⁹R¹⁰, reacting a compound of formula (VI) with a compound of formula (VII): 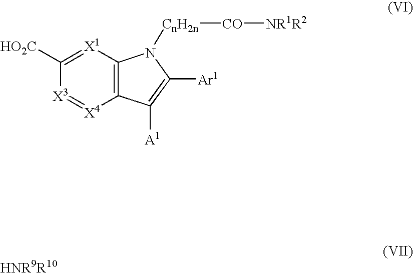
wherein X¹, X³, X⁴, A¹, Ar¹, n, R¹, R², R⁹ and R¹⁰ are as defined in claim 1, essentially in the same manner as general process (B);
(D) where the (aza)indolyl nitrogen atom is suitably protected, reacting a compound of formula (VIII) with a compound of formula (IX): 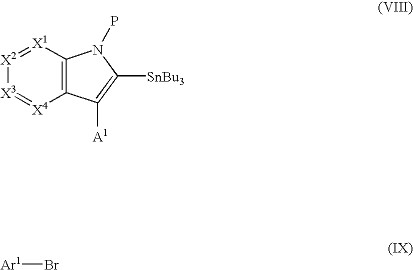
where P is a suitable protecting group and wherein X¹, X², X³, X⁴, A¹ and Ar¹ are as defined in claim 1, effected in the presence of a Pd(0) catalyst, a suitable ligand and a salt in a suitable solvent at a temperature between 20° C. and the reflux temperature of the solvent; or
(E) where X² is CR³ and R³ is C(O)NR⁹R¹⁰ and R⁹ is SO₂R¹¹, reacting a compound of formula (VI) with a compound of formula (X):
R¹¹O₂S—NHR¹⁰ (X)
wherein X¹, X³, X⁴ , A¹, Ar¹, n, R¹, R², R¹⁰ and R¹¹ are as defined in claim 1, in the presence of an activator and/or a dehydrating agent in a suitable solvent.

9. A method of inhibiting hepatitis C virus polymerase which comprises administering to a subject in need of such inhibition an effective amount of a compound of according to claim 1 or a pharmaceutically acceptable salt thereof.

10. A compound according to claim 2, wherein:
X² is CR³;
R¹ is hydrogen, C₁₋₆ alkyl or CH₂Het;
R² is hydrogen or C₁₋₆ alkyl;
or alternatively R¹, R² and the nitrogen atom to which they are attached form a five- or six-membered heteroaliphatic ring, which ring optionally contains one additional oxygen atom or a group NRS, which ring is optionally substituted by (CH₂)₀₋₃NR^{t}R^{u}; and
Q¹ is halogen, hydroxy, C₁₋₄ alkyl, or C₁₋₄ alkoxy.

11. A compound according to claim 10, wherein:
R³ is CO₂H, heteroaryl, or C(O)NR⁹R¹⁰;
R⁹ is hydrogen or methyl;
R¹⁰ is SO₂R¹¹; and
R¹¹ is C₁₋₄ alkyl, phenyl, benzyl, trifluoromethyl, CH₂CF₃, methoxyphenyl, pyridyl, thienyl, or (CH₂)₂ phenyl.
